(19) United States
(12) Patent Application Publication
(10) Pub. No.: US 2008/0020405 A1
(43) Pub. Date: Jan. 24, 2008

(54) Protein binding determination and manipulation

(76) Inventor: Dennis C. Mynarcik, Huntington, NY (US)

Correspondence Address: Thomas M. Saunders;Brown Rudnick Berlack Israels LLP, BOX IP, One Financial Center, Boston, MA, 02111, US

(21) Appl. No.: 11/796,898

(22) Filed: Apr. 30, 2007

Related U.S. Application Data
(63) Continuation-in-part of application No. 10/620,491, filed on Jul. 16, 2003, now abandoned.
(60) Provisional application No. 60/396,428, filed on Jul. 17, 2002.

(51) Int. Cl. G01N 33/53

(52) U.S. Cl. 435/7.8

(57) ABSTRACT

This invention addresses method for determining protein binding sites, molecules which bind to such sites and molecules which inhibit binding to particular protein binding sites. Also considered are conjoined molecules for enhanced binding to specific protein binding sites.

### CROSS REFERENCES TO RELATED APPLICATIONS

This patent application claims priority to provisional patent application No. 60/396,428, filed in the U.S. Patent and Trademark Office on Jul. 17, 2002, and to U.S. patent application Ser. No. 10/620,491 filed Jul. 16, 2003, the entire contents of each is incorporated herein by reference.

### FIELD OF THE INVENTION

This invention addresses method for determining protein binding sites, molecules which bind to such sites and molecules which inhibit binding to particular protein binding sites. Also considered are conjoined molecules for enhanced binding to specific protein binding sites.

### BACKGROUND OF THE INVENTION

Reference is made to Phage Display of Peptides and Proteins: A Laboratory Manual, Ed. Kay et al., Academic Press, Inc.; “Directed evolution of novel binding proteins,” U.S. Pat. No. 5,837,500 (Ladner et al.), “Engineering affinity ligands for macromolecules,” U.S. Pat. No. 6,326,155 3 (Maclennan et al.), “Methods for rapidly identifying small organic molecule ligands for binding to biological target molecules” (Wells et al.) U.S. Pat. No. 6,335,155, “Protein tyrosine kinase agonist antibodies,” Bennett et al. U.S. Pat. No. 6,331,302, and “Monovalent phage display,” U.S. Pat. No 5,821,047 (Garrard et al.) the teachings of which are incorporated herein by reference. For clarity, the teachings of all patents, journals, texts and publications noted herein are incorporated by reference.

Attention is drawn to Cwirla, et al. “Peptides on Phage: A Vast Library of Peptides for Identifying Ligands,”. Proc. Nat'l Acad. Sci., USA 87:6378-6382 (1990). Cwirla discloses a method of panning for peptides. This method, however, will necessarily exclude that fraction of peptides with low affinity for target protein expressed as a surface patch.

Attention is drawn to Canadian application.2377371 (PCT Pub No. 2001/002440) to Dennis et al. “Fusion Peptides Comprising A Peptide Ligand Domain And A Multimerization Domain” (“Dennis”). Dennis is not applicable to the instant invention, in part, because Dennis uses a classical bacteriophage peptide display and panning method to discover peptides that bind to one known protein molecule with high affinity and, after the fact, the peptide is linked in a fusion protein construct to a multimerization domain, such as an immunoglobulin or leucine-zipper to bring an additional chemical moiety to the known protein molecule. This methodology is limited to identifying only high affinity peptides. Fusion is subsequent to the bacteriophage peptide display selection process and the multimerization domain is to attract an unrelated chemical entity to the site on the known protein molecule as opposed to the current invention in which the known target region is an inseparable part of the target protein molecule.

### SUMMARY OF THE INVENTION

In one embodiment this comprises a method of obtaining a primary-result peptide having at least one binding domain that binds a predetermined dynamic target material at a non-active site wherein said dynamic target material has at least two conformational energy-minima states comprising:

(a) accessibly-conformationally restraining said dynamic target material in substantially a single conformational energy-minima state

(b) affinity-exposing said accessibly-conformationally restrained single conformational energy-minima dynamic target material to a peptide library comprising inquiry-peptides and identifying peptide which associate with the target with sufficient affinity to withstand washing at least about 4 times in rapid succession with a standard buffer containing physiologically balanced salt solution and a non-ionic detergent (<0.1% v/v) (“peptide hits”).

(c) affinity-exposing said accessible conformationally-restrained single conformational energy-minima state dynamic target material to said peptide library wherein said single conformational energy-minima state is substantially a single energy-minima state other than the state of step (a) and identifying peptide-hits; and

(d) selecting at least one peptide-hit that inhibits target function by other-than-competitive inhibition the target material, which peptide-hit being a primary-result peptide.

This invention further includes a method of obtaining a primary-result peptide having at least one binding domain wherein said binding domain is a low affinity binding domain comprising:

(a) preparing a target polypeptide, as a fusion protein having a known target region and an inquiry target region wherein the known target region is linked to the inquiry target region by a flexible linker;

(b) preparing a tandem peptide display library where said tandem peptides comprise
(i) a known peptide element having a binding domain of low affinity as to said known target region said element connected to
(ii) a flexible linker said flexible linker connected to
(iii) an inquiry peptide sequence

(c) affinity exposing said target protein to said peptide library;

(d) identifying tandem peptide-hits;

(e) identifying said inquiry peptide sequence of said tandem peptide hit as a primary result peptide. In a particular embodiment the method further includes the known target region of (a) comprising an SH3 domain and the known peptide of step (b)(i) comprising a protein-rich SH3 binding domain having an affinity for the known target region with an affinity in the range of 100 micromolar, so as to be of sufficiently low affinity to substantially dissociate from the known target region after washing at most about 4 times in rapid succession with a standard buffer containing physiologically balanced salt solution and a non-ionic detergent (<0.1% v/v). In a particular embodiment, the method further comprises the flexible linker of step (b)(ii) being a short peptide.

In a yet further embodiment the invention comprises a method of obtaining a primary-result peptide useful in inducing formation of activated-like multiprotein complexes bridging two partner polypeptides comprising:

(a) anchoring to a substratum a target polypeptide having a known dimerizable target region, said anchoring being at a location other than said target region and assembling the multiprotein complex, as a ternary complex, by adding a partner target polypeptide and cognate-like accessory polypeptide (such as a hormone) which bridges the two partner polypeptide targets (such as extracellular hormone binding domains of membrane receptors acting as target polypeptides);

(b) exposing said substratum anchored activated-like multiprotein complex to a phage peptide display library and

(c) selecting phage that bind the assembled protein-protein complex with sufficient affinity to withstand washing four times in rapid succession with a standard buffer containing physiologically balanced salt solution and a non-ionic detergent (<0.1% v/v)

(d) selecting from among said complex binding phage a phage that when added to a system containing a substratum anchored target polypeptide and a partner target polypeptide, is capable of inducing the formation of the multiprotein complex such that the two target polypeptide partners become associated in the absence of the accessory polypeptide, said phage bearing a primary result peptide.

In a further embodiment this invention comprises a method of preparing an enhanced peptide display library comprising preparing a tandem peptide display library having a known target region and an inquiry target region where said tandem peptides comprise
(i) a known peptide element having a binding domain of low affinity as to said known target region said element connected to
(ii) a flexible linker said flexible linker connected to
(iii) an inquiry peptide sequence
(iii) wherein said inquiry peptide sequence is further connected to a bacteriophage structural protein, as well as the library of this method. Particular attention is drawn to an enhanced peptide display library comprising a tandem peptide display library having a known target region and an inquiry target region where said tandem peptides comprise
(i) a known peptide element having a binding domain of low affinity as to said known target region said element connected to
(ii) a flexible linker said flexible linker connected to
(iii) an inquiry peptide sequence
(iii) wherein said inquiry peptide sequence is further connected to a bacteriophage structural protein.

### BRIEF DESCRIPTION OF THE DRAWINGS

FIG. 1 is a conceptual drawing of an Erythropoietin receptor (EPOR) with hormone binding domain, amino terminal domain, hormone binding pocket, and carboxyl terminal domain.

FIG. 2 is a conceptual drawing of Erythropoietin (EPO) with a high affinity surface and a low affinity surface.

FIG. 3 is a conceptual drawing of the association of the high affinity surface of an EPO molecule with the hormone binding pocket on an EPOR (an initial event).

FIG. 4 is a conceptual drawing of EPORs anchored on a membrane such that they can only diffuse laterally or rotate in the plane of the membrane. The straight arrow indicates lateral diffusion and the curved arrow indicates rotational diffusion.

FIG. 5 is a conceptual drawing of EPO-EPOR binding. Once the high affinity EPO surface binds to the first EPOR, the low affinity EPO surface is positioned with a narrow two-dimensional plane. Because the unoccupied EPORs can only diffuse laterally or rotate in that narrow plane, they can easily engage low affinity EPO surface, forming the activated complex.

FIG. 6 is a conceptual drawing of LZHRs. LZHRs are short helical peptides with one face of the helix composed of the amino acid leucine (grey), which has a hydrophobic (water-avoiding) side chain. When two LZHRs are in close proximity the two leucine faces zip together (right), to be shielded from water.

FIG. 7 is a conceptual drawing of the attachment of a short LZHR to EPOR by a flexible linker peptide, the formation of the EPOR*-EPO-EPOR* complex can be effectively achieved in a cell-free environment.

FIGS. 8 through 17 depict the amino acid side chains to be mutated to the alanine methyl group in the panel of mutants used to identify peptides from a sub-library selected by an initial panning procedure associated with a targeted EOPbp sub-domain.

### DETAILED DESCRIPTION OF THE INVENTION

This invention will be better understood with reference to the following definitions:

A tandem peptide display library shall mean a library in which specific peptide structures are expressed ion phage, typically on the amino-terminus of a subset of the pIII molecules of the M13 bacteriophage. While the pIII molecule is often used, other bacteriophage surface proteins are also able to serve as a platform for peptide display, such as the pVIII molecule. Other proteins as well can also be employed. The display peptide consists of three elements, (i) a combinatorial peptide or inquiry peptide sequence of four or more amino acids flanked by two cysteine residues, (ii) a linker amino acid sequence that connects the combinatorial sequence to (iii) a constant or known peptide sequence that is in turn linked to the amino-terminus of, in one embodiment, the pIII molecule, by a flexible linker peptide. Flanking the combinatorial inquiry sequence by two cysteines allows the cysteines to form a disulfide bond to arrange the combinatorial sequence in a loop structure to reduce the number of conformational states they can adopt. The linker peptide sequence can vary in length and flexibility and can, in some embodiments, be composed of two or more glycine residues to create a flexible linker. In another embodiment, the linker can be a rigid alpha-helix, flanked by two glycine residues on either end or both ends. The constant or known peptide sequence is a peptide that binds to the protein domain or known target region with a weak affinity (in the range of about 10s to 100s of micromolarm and more particularly 5 to 500 micromolar).

Known peptide element shall mean: a peptide sequence with a weak affinity (about 10s to 100s of micromolar and more particularly 5 to 500 micromolar) for its complimentary known target region. The known peptide element is present on each member of a tandem peptide display library and serves to bring each member of the library to the target by virtue of its weak affinity for the known target region with which the target protein molecule is adapted.

Known target region shall mean a protein interaction domain such as the SH3 domain that has a weak affinity for peptide sequences containing proline residues. An SH3 domain can be linked to the target protein molecule by a linker peptide as described in the description of the tandem peptide library. The known target region can also be a site on a target protein molecule that binds an inquiry peptide. Often such inquiry peptide will have been discovered in a previous iteration of the panning procedure. This makes the identified inquiry peptide a new known peptide element in a new library.

Flexible linker shall mean a peptide sequence that contains two or more glycine residues in addition to other amino acids such as serine. The glycine sequence can also be interrupted by helical sequences that limit the flexibility to one end, the other or both ends. The length and flexibility of the linker defines the volume within which the structure attached to the linker, such as the known target region or the inquiry peptide can reside. The longer the linker the greater the volume and the longer it will take the two binding partners to reacquire each other.

Inquiry peptide shall mean a combinatorial or hypervariable peptide sequence in which substantially all of the possible combinations of amino acid sequences are represented.

Without being bound by any particular theory it is believed that a target protein's surface may be conveniently considered as having has two regions. The first is an active site. The second region is the rest of the molecular surface. The active site is usually an invagination on the target's surface, making a pocket into which a substrate or a hormone binds, for enzymes or receptors, respectively. The pocket nature of the active site provides a three-dimensional surface, greatly enlarging the surface area of contact between the bound (binding) molecule and the target. In this abstraction, the remaining volume of the protein molecule serves as a scaffold for the formation of the pocket. In contrast, the rest of the target protein's surface can be approximated as the convex surface of a sphere.

Again without being bound by any particular theory it is believed that perturbing structural arrangements on the protein surface can cause configurational changes in the structure and function of the active site. Currently, much of the drug discovery effort in bio-pharma is directed at the active sites of target proteins. This is likely a result of the active site being the region of the target protein where there is intensive structural knowledge. The structure of an effecting hormone is often known in high resolution and the structures of the substrates and products, as well as the enzymatic mechanism, are often well established. There is also structural information available for a large number of protein targets. These two datasets appear to fuel the development of structural mimics that dominate the drug discovery pipeline. While structure mimics can be effective for their designated target, they are also potential sources of negative side effects. This factor contributes to the high rate of compound failure in pre-clinical and clinical trials. Therefore, the industry has a significant interest in identifying non-active site surface loci on the target protein molecule to which the drug discovery apparatus can be directed. As there is no technology or computer algorithm reliably able to identify function-altering sites on a protein's surface, an empirical approach is a useful alternative to identify them.

Current pharmacology prefers to limit the size of drug molecules to about 500 Daltons or less in an effort to limit side effects. While not unreasonable, such limitation necessarily excludes unique chemical entities composed of carbon, oxygen, nitrogen, hydrogen and sulfur, with molecular weights ≦500 Daltons. This group has been estimated to be about 10⁶² compounds. This is more than the number of particles in the known universe, making an unguided synthetic chemical approach impractical for hunting down useful compounds. Proteins, however, are allosterically regulated by other proteins and peptides, via protein-protein interactions. Peptides can achieve structures that are complementary to any surface patch on a target protein. Thus, bacteriophage peptide display is a technological approach that can be applied to discovering non-active site functional patches on target protein molecules.

It has now been discovered that, to confine the search to patches in the 500 Dalton range, cysteine-constrained peptide-loops created by flanking combinatorial amino acid sequences of four to eight amino acids in length with two cysteine residues can be used. Peptide loops four to eight amino acids long can cover patches of 2-8 nm2, within which a 500 Dalton molecule could bind. However, when peptide display has been used to identify sequences that bind to and alter the function of protein molecules the results have been limited to sequences that bind to the active site. This is a function of the process of selecting peptides (panning) and the target—peptide interfacial surface area. In panning the target is immobilized and incubated with the combinatorial peptide display library, loosely bound material is removed by washing steps, and the tightly bound phages are eluted by weak acid. The eluted phages are re-grown and the panning process repeated three to five times. This sequential process selects for a small number of peptide motifs with a high affinity for the target. These peptides always bind to the target's active site. An explanation for this is that the interfacial surface area between the peptide and the target is two to three times larger in an active site that the more two-dimensional interface available on the remaining non-active site surface. The greater the interfacial surface area the greater the number of molecular contacts and the higher the affinity of the peptide for the target, accounting for the dominance of peptides that bind to active sites. The dilemma is that the loci on the non-active site surface of target protein molecules that are in the 2-8 nm2 range will have a much lower affinity for complementary peptides.

Within the combinatorial library, four populations of peptides exist: #1 a very small fraction with high affinity for the active site; #2 a larger fraction with moderate affinity for surface patches; #3 a still larger fraction with low affinity for surface patches; and #4 the bulk of the library that has no meaningful affinity for the target. Within the panning procedure, after the library and the combinatorial library have come to equilibrium the material that can be aspirated away contains fraction #4. The container with the immobilized target and associated phages is then washed repeatedly, removing all of fraction #3, a portion of fraction #2 and very little of fraction #1. In subsequent panning rounds the members of fraction #1 come to proportional domination, which is why peptides that bind to the active site dominate the yield of panning.

Capturing a member of the peptide display library by virtue of its capacity to bind to a surface patch on the target relies, in part, on the affinity of the interaction between the peptide and the surface patch being greater than or equal to some threshold affinity. The metric for quantifying affinity is the dissociation constant (K_{d}), which is the concentration of the peptide at which 50% of the peptide is bound to the available surface patch. The K_{d} is also defined as the ratio of the rate constant of association (kₒₙ) and the rate constant of dissociation (k_{off}). When the mixture of target and peptide is at equilibrium the ability to capture all of the bound structures is defined by the k_{off}. If the k_{off} is faster than some threshold k_{off}, the peptide will be washed away and it will not be captured by panning.

With a view to these gives, fractions #2 and #3 are of interest in that they contain moderate to low affinity peptides. As the affinity diminishes, the number of different peptide sequences increases and the more completely the target's non-active site is covered. As the #2 fraction has fewer members, albeit of higher affinity, than fraction #3, the probability that it will contain peptides that interact with function altering sites is much lower, in that the number of sites through which function can be altered is a very small fraction of the total number of potential sites. One advantage of the present invention is to determine if such a site exists. This, in turn, leads to an effort to have all sites interrogated, making the contents of fraction #3 the highest value.

One way to capture the members of fraction #3 is to increase the surface area of contact between the fraction members and the target. This is done indirectly with the Anglerfish technology. Combinatorial peptide loops are linked by a short peptide to a constant peptide sequence that is in turn linked to a bacteriophage surface protein. The constant peptide has a weak affinity for a protein domain that is linked to the target by a short peptide. Weak affinity can be defined functionally as an affinity that will result in the dissociation of the ligand during the span of repeated washing over a span of 20 minutes. The affinity of the constant peptide for the protein domain is within in the range of that of the fraction #3 peptides for the target surface. This is done so that if the only interaction is between the constant peptide and the protein domain linked to the target, the phage will be lost during the washing phase.

In order for a phage to be captured one of the two associative events—either the interaction between the combinatorial loop and a target surface site or the constant peptide and the linked protein domain—will have to exist at substantially all times. The rate of dissociation for either binding pair is slower than the rate of association of either binding pair. This places limits on the length and flexibility of the linking structures. The linker connecting the combinatorial peptide to the constant peptide defines a volume within which the combinatorial peptide can be found relative to the constant peptide. The linker connecting the protein domain to the target similarly defines a volume within which the protein domain can be found relative to the target.

The greater the accessible volumes, the longer it will take the unbound pair to reacquire each other. The longer it takes for the unbound pair to reacquire each other the greater the chance that the bound pair will dissociate and the phage will be lost. The shorter the linkers, the greater the probability that one of the two binding events will always exist, facilitating capture, but this will result in a smaller fraction of the target's surface area that is accessible to the combinatorial peptide.

An advantage of the anglerfish technological approach to discovering functional sites on the surface of the target protein is its ability to interrogate the entire surface of the target molecule. When the dimensions of the target protein molecule are in excess of the area that can be interrogated by the linkers employed, a secondary strategy is able to extend the anglerfish technology to completely investigate the target's surface. In the secondary strategy a set of new libraries is generated in which the constant peptide of the library is replaced with a subset of combinatorial peptide loops discovered in the initial anglerfish panning. These peptides have affinities generally insufficient to be retained following washing when used independently, but they have generally sufficient affinities to bring the phage to the target for a duration defined by their k_{off}. Thus, there will be a number of independent new libraries constructed, each of which have the constant peptide replaced with a peptide discovered in the initial anglerfish panning that now becomes the new constant peptide. This is in turn linked to the combinatorial peptide loops. In this way the anglerfish technology provides a means of “walking” across the entire surface of the target.

Ordinarily, few of these peptides can work as tools due to their low affinity. It would require a very large abundance of them to be used for any type of screening. By one strategy, in order for the peptide to have a sufficient affinity it can be placed in the position in the phage of the constant peptide, linked to the combinatorial peptide loop by a short linker with limited flexibility. This will provide the ability to select a small number of phage that have the functional peptide supplemented with another peptide that binds to an adjacent site on the target's surface for enhanced affinity.

I. Protein Topology Affixation Protocol

One embodiment of the present invention is a protein topology affixation process. The practice of this invention encompasses a process for discovering peptides from combinatorial display libraries that associate with a target enzyme at a non-active site location, and, through such associations, restrict a site specific enzyme from progressing through the changes in conformation necessary for completion of the catalytic cycle peculiar to that enzyme, and in this way inhibit the enzyme's activity by an other-than competitive mechanism (substrate-mimicry).

One use of this process is in drug-development. This process targets the massively-diverse chemical topology of protein surfaces in order to develop drug molecules that are chemically complementary to strategic surface loci with the capacity to restrict the target's conformational dynamics. In addition this process identifies drug molecules with significantly improved selectivity for individual members of large protein families and develops drug molecules with significantly reduced negative side-effect profiles resulting from improved selectivity.

Conventional target-directed drug discovery has two limited chemical-space data sets available for the design of libraries from which lead compounds are selected, i.e., the structure of the native substrate/ligand and the topology of the target's active-site. The exploitation of both of these data sets has driven the drug-discovery engine of the biotechnology industry.

In a departure from prior design, by the present invention targets are immobilized conformationally prior to ligand determination. In one example of a protocol for enzyme inhibitors (protein tyrosine kinase as example of such enzyme) target immobilization is accomplished as follows:

Targets are immobilized using a c-terminal extension consisting of the peptide sequence (G L N D I F E A Q K I E W H E), unless the c-terminus is integral to target mechanism of action. In the case where the c-terminus of the target is integral to the target's action the peptide sequence can be added to the n-terminus. This peptide sequence is a substrate for in vitro biotinylation using a commercially available enzyme, biotin protein ligase, from Avidity, Denver, Colo. The biotin-derivatized target is then immobilized on avidin- or streptavidin-coated microtiter plates.

Given the mechanism of target action, two extremes of conformation are identified.

In one extreme the kinase molecule is closed around a non-hydrolysable ATP analog. In the other extreme, the kinase molecule is open with the ATP binding pocket empty.

This process entails affinity isolation of display peptides. In a specific embodiment a bacteriophage peptide display library is applied to the target immobilized in one of the two conformational extremes. Phage that bind to the target are then isolated. The process is repeated with the target held in the other conformational extreme.

Phage characterization is a next step. This includes identification of display peptides specific to one conformational state. Phage clones that associate with the target, held in one of the two conformational extremes are assessed for their ability to bind to the target when it is held in the other conformational extreme. This step identifies those phage clones that bind exclusively to only a single target conformational state. Those single conformational binding phage clones bind to the target at potential function-altering target surface domains. Those phage that bind exclusively to one conformational target state are assessed for their ability to inhibit the activity of the target. Those single conformational binding phage that inhibit the activity of the target are prepared as peptides and assessed. Peptides that perform as the intact phage are advanced. Advanced peptides are assessed for the type of target inhibition, i.e., competitive or other than competitive inhibition, using classical enzyme kinetic analysis.

In one embodiment, peptides that inhibit by other than competitive mechanisms are optimized by affinity maturation to optimize the peptide sequence for binding affinity. The optimized peptides are re-assessed to confirm that target inhibition characteristics are unchanged (or superior). The peptides thus selected are particularly useful in target binding assays used to screen chemical libraries for interaction with the target domains with which the peptide associates. A complimentary use is to determine the chemical-space defined by the peptide's chemistry, employing computational chemistry, in order to design focused combinatorial chemical libraries.

The peptides so identified are also termed protein dynamics modulators (PDMs). PDMs bind to a target, stabilizing one conformational state, preventing progression to other states. PDMs bind to non-active site, functional epitopes on the target's surface (non-competitive/uncompetitive). PDMs modulate target function through restricting the target's structural dynamics. They define the chemical space of the functional epitopes, guiding chemical library design, and are useful in high-throughput screening displacement assays to generate or validate lead compounds.

As noted above PDMs are selected from phage peptide display libraries in a two stage process. First, phage are selected for the ability to bind to immobilized target molecules that are held in one conformational state. Then, phage, identified in stage one, are further selected for the ability to hold the target in the chosen conformational state, preventing the transition to other conformational states. Phage that restrict the target to a single conformational state, and through that restriction inhibit target function, encode for peptides that comprise PDMs.

Examples of proteins usefully restricted in conformational state in the practice of this invention include, the abl tyrosine kinase (as well as other kinases), Acetyl CoA carboxylase 2, and other enzymes with particular reference to those of important physiological regulatory significance.

### Protocol for Enzyme Inhibitors

### abl Protein Tyrosine Kinase Example

Target Immobilization:

Targets are biotinylated and immobilized on streptavidin-coated microtiter plates. The target sequence is modified on the c-terminus to include the sequence (G L N D I F E A Q K I E W H E), an optimized substrate for biotin protein ligase. The modified target is expressed in a eukaryotic expression system. The c-terminal extension is derivatized with a biotin using biotin protein ligase (Avidity, Denver, Colo.). The biotin-derivatized target is then immobilized on streptavidin-coated microtiter plates.

Using knowledge of the mechanism of target action, two extremes of conformation is identified. At one extreme: is the kinase molecule closed around a non-hydrolysable ATP analog. At the other extreme: the kinase molecule open with the ATP binding pocket empty.

Affinity Isolation of Display Peptides:

A bacteriophage peptide display library is applied to a target immobilized in one of the two conformational extremes. Those phage that bind to the target are isolated. Next, the process is repeated with the target held in the other conformational extreme.

Phage Characterization:

Identification of display peptides specific to one conformational state:

Phage clones that associate with the target, held in one of the two conformational extremes are assessed for their ability to bind to the target when it is held in the other conformational extreme to identify those phage clones that bind exclusively to only one target conformational state. Those phage clones bind to the target at potential function-altering target surface domains. Those phage that bind exclusively to one conformational target state are assessed for their ability to inhibit the activity of the target. Those phage that inhibit the activity of the target are prepared as peptides. Those peptides that perform as the intact phage are advanced. Advanced peptides are assessed for the type of target inhibition, i.e., competitive or other than competitive inhibition, conveniently, using classical enzyme kinetic analyses. Peptides that inhibit by other than competitive mechanisms are optimized by affinity maturation to optimize the peptide sequence for binding affinity. The optimized peptides are re-assessed to determine if target inhibition characteristics have changed. Those peptides that have retained their inhibitory characteristics are prepare as conjugates. These conjugates facilitate in vitro target detection and are used in target binding assays.

Peptide sequences are analyzed by computational chemistry for the design of focused combinatorial chemical libraries. These libraries are screened for target binding in peptide displacement assays.

II. Low Affinity Peptide Display Protocol

Another aspect of this invention uses structural inquiry in discovering and isolating peptides from combinatorial display libraries that associate with a target protein at locations with affinities too low to withstand conventional washing. This technique takes advantage of the multiplicative affinity of conjoined peptides and/or molecules. Low affinity target-interacting peptides from a peptide display library are captured by linking a random display peptide sequence to a constant peptide sequence that has low affinity for an additional protein domain linked to the target protein as a fusion protein by a flexible linker. The affinity for the two (or more) linked peptides is the product of their individual affinities for their respective protein domains. A constant peptide sequence is selected for binding additional protein domain(s) with an affinity low enough to prevent binding to be maintained without an additional binding contribution from the random display peptide. The strategy of employing a binary library identifies peptide sequence families in the random display peptides that otherwise go undetected by conventional panning approaches and the like.

In the process of this aspect of the invention a target is prepared. It is useful to prepare the target protein as a fusion protein such that the target protein is linked by a flexible linker peptide to a protein domain (the bait) known to bind a specific peptide sequence with low affinity. A specific example target is (abl) fusion protein construct. This construct has an SH3 domain linked to the amino-terminus (or to the carboxyl-terminus) of the target (abl catalytic domain) by a flexible linker peptide (the flexible linker peptide is varied in length to accommodate to varying target sizes).

A library display is then employed. The peptide display library is used so that the constant low-affinity peptide is linked by a short flexible sequence to the random display peptide sequence. In this embodiment one peptide display library consists of two structural peptides linked by a flexible linker peptide sequence. One structural peptide is held constant (e.g., proline-rich SH3 binding peptide sequence). The constant sequence is linked by a short flexible linker peptide with the random peptide display sequence. The constant sequence is chosen for low affinity binding (high micromolar) to the constant domain.

Isolated low affinity peptides are then used as basis for defining or developing higher affinity analogues. In some cases a series of single amino substitutions are made resulting in higher affinity analogues. Other affinity increasing techniques are known in the art. Resulting analogues with increased affinity are useful as peptides that associate with a target enzyme at active or non-active site locations, and, through such associations, restrict a site specific enzyme.

III. Protein-Protein Interaction Inhibitors and Method of Use.

Yet another one embodiment of this invention includes a process for the discovery of molecules from combinatorial peptide display libraries that block protein-protein interaction, particularly as used in in vitro discovery systems. Molecules which block protein-protein interaction by competing for a protein-protein contact surface are useful in defining “surfaces” which induce therapeutic protein-protein interaction.

In one embodiment, the present method identifies molecules that block specific protein-protein interactions. Useful points of inquiry are molecules that, (i). are validated as contributing to disease, (ii) are composed of two identified protein targets, (iii). are mediated by structurally defined protein-contact surfaces, and (iv). are difficult to assemble as an in vitro assay in a high-throughput screening environment.

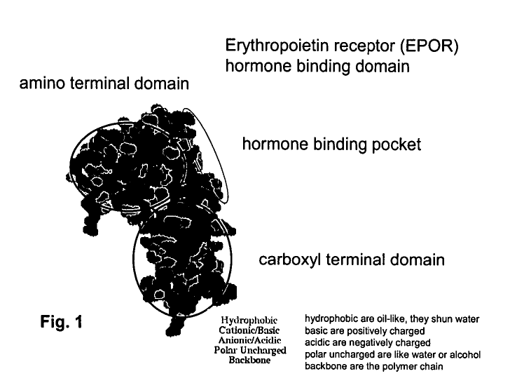

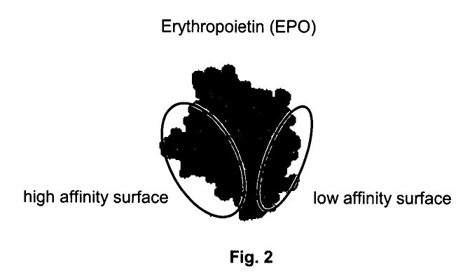

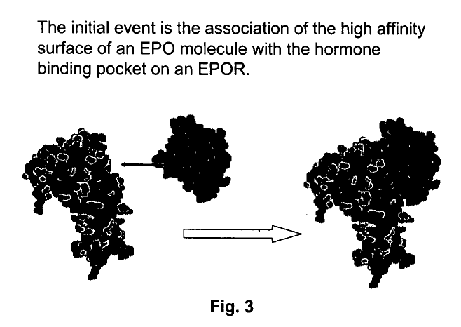

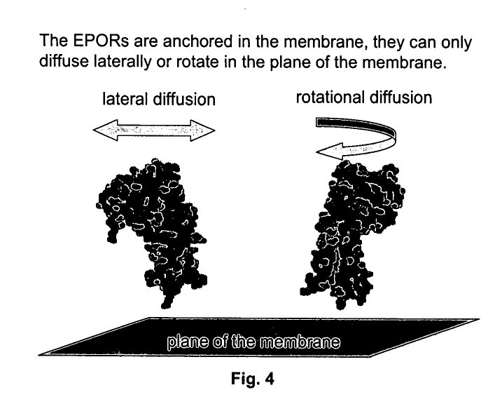

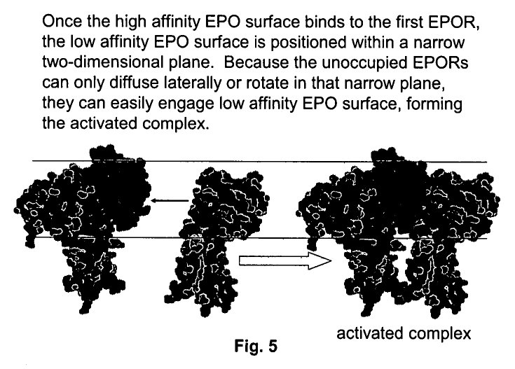

The dynamics of EPOR activation by EPO, as shown in FIG. 1, can be reduced to a two step process (EPO itself has a high affinity surface and a low affinity surface as shown in FIG. 2)
EPO binds to one EPOR (FIG. 3)
1. A second EPOR is recruited to the EPOR-EPO complex creating the EPOR-EPO-EPOR activated complex. The above-noted technique is employed to select PDMs that block the transition from the EPOR-EPO state to the EPOR-EPO-EPOR state and to select PDMs that bind to the EPORs only in the EPOR-EPO-EPOR complex
   The PDMs selected in this first example come with inherent advantages that are a direct result of the design of the secondary screening process. Both the PDMs and the EPOR sites to which they bind are chemically and conformationally defined. These comprise target/configuration/binding information useful in the design of the chemical libraries used in drug discovery. As shown in FIG. 5, the activated complex, the PDM binding sites on one EPOR are opposite to and in close proximity to PDM binding sites on the other EPOR. Enhanced binding of PDMs is achieved (i) optimizing initially identified PDMs and then linking two or more PDMs together. Such a linked molecule comprises an activated complex.

In a particular embodiment of this invention one selects PDMs that bind to the EPORs only in the EPOR-EPO-EPOR complex. Note that it is very difficult to form the activated EPOR-EPO-EPOR complex in a cell-free environment. This is because the two EPORs that come together to form the activated EPOR-EPO-EPOR complex are not restricted to the two-dimensions of the membrane, but are free to diffuse in three dimensions, requiring the second EPOR to be present at extremely high concentrations. EPORs anchored to a membrane are shown in FIG. 4 One approach to overcoming this difficulty is to link an additional structural feature, with a low affinity attraction for itself, to the end of the EPOR (EPOR*).

The affinity for the formation of the EPOR*-EPO-EPOR* complex is the product of the affinities for the two associative events, i.e., the low affinity EPO/EPOR binding is multiplied by the low affinity binding of self-associating linked structure, note FIG. 5.

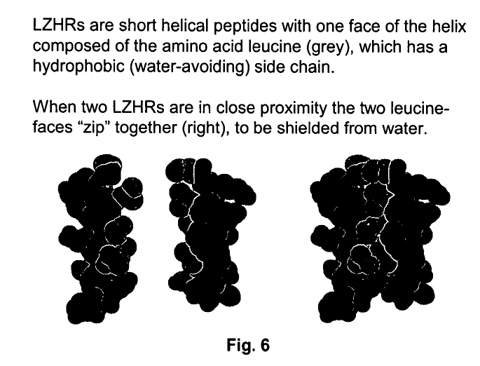

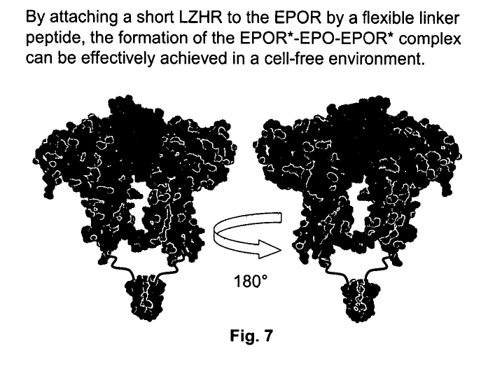

The leucine-zipper heptad-repeat (LZHR) is useful for the self-associating linked EPOR*-EPO-EPOR* structure. When two LZHRs are in close proximity the two leucine faces “zip” together to be shielded from water as shown in FIGS. 6 and 7. The process of selecting phage for candidate PDM identification has two phases,
1. The selection of all phage that bind to the activated EPOR*-EPO-EPOR* complex is a first phase
2. Identification of phage selected in the first round that can induce the formation of an EPOR*-EPOR* complex in the absence of EPO is the second phase.

Note that by attaching a short LZRH to the EPOR by a flexible linker peptide, the formation of the EPOR*-EPO-EPOR* complex can be effectively achieved in a cell-free environment.

A significant embodiment of the invention is the process comprising two phases performed in sequence. In the first phase, one member of a protein-protein interacting pair is immobilized such as on a substrate. Next, display peptides that associate with the target are selected. Selection usefully employs the technique of panning (this approach is compatible with the anglerfish binary screen technology but other selection techniques are contemplated within this invention). Those display peptides selected in the first phase are then passed through a second phase screen. The second phase screen consists of screening the entities selected in the first-phase panning against a family of target site-directed mutants in which at least one and in some embodiments all charged amino acid residues residing on the inter-protein contact surface have been changed to the amino acid alanine. First-phase selectants that associate with the inter-protein contact surface are identified by their ability to associate with the wild type (non-mutated) target and all but a subset of mutant target molecules. The subset of mutants to which the first-phase selectant fails to bind identifies the target inter-protein contact surface loci to which the selectant binds.

More specifically in Phase One a target protein is prepared with an amino or carboxyl terminal extension useful for immobilizing the target in vitro so that target function is largely unperturbed and substantially the full target surface area is accessible to the media. Panning technology collects members of a combinatorial peptide display library that specifically associate with the target.

The target (e,g., erythropoietin receptor extracellular hormone binding domain (ERHBD)) is generated with amino-terminal peptide extension (G L N D I F E A Q K I E W H E). The lysine residue (K) is biotinylated enzymatically (ERHBD*) and the construct is immobilized on avidin-coated plastic plates. Proper target folding is established by determining epo binding. A combinatorial peptide display library, preadsorbed on avidin coated plates saturated with biotin, is then applied to the immobilized ERHBD*, and those elements of the library associating with the ERHBD* are collected. The collected elements are “phase-one selectants”.

Immobilization technology is exemplary of the approach. Other techniques that capture the target without altering its surface structure are adequate.

In Phase Two a family of target protein constructs in which charged amino acid residues present on the protein-protein contact surface are individually mutated to the amino acid alanine. The wild type (non-mutated) and the alanine mutant constructs are then immobilized as an array in microtiter plates and the Phase One selectants are screened for binding to the array. Those Phase One selectants that bind to the protein-protein contact surface are identified by their binding to the wild type and all but a subset of the mutant constructs. Those mutants that exclude the Phase one selectants identify the surface locus to which the selectants bind.

In the ERHBD-epo-ERHBD complex, the carboxyl-terminal fibronectin type III (FNIII) domains of the two ERHBD are positioned opposite each other. The charged amino acid residues located within the protein-protein contact region are R130, D133, E134, R141, R171, E173, E176, R178, E180, and R187 (R=arginine (+), D=aspartic acid (−), and E=glutamic acid (−)). Ten individual ERHBD* mutants are constructed in which each of the listed charged amino acid residues are mutated to alanine (this is a classical strategy used to assess the role of specific amino acid side chains in biochemical processes). The wild type ERHBD* construct and each of the ERHBD* alanine-mutants are then immobilized as an array in avidin-coated microtiter plates, i.e., wild type in column 1, R130A in column 2, D133A in column 3, E134 in column 4, R141 in column 5, R171 in column 6, E173 in column 7, E176 in column 8, R178 in column9, E180 in column 10, R187 in column 11, and wild-type in column 12. The individual Phase One selectants are then dispensed into individual rows and their ability to bind to the immobilized array of ERHBD* constructs are assessed. Those Phase One selectants that bind equally to all of the ERHBD* constructs in the row bind to ERHBD regions that are outside of the protein-protein contact region. Those Phase One selectants that bind to the wild type and all but one or a subset of the alanine mutants are identified as binding to a locus within the protein-protein contact region. Furthermore, the specific alanine mutant(s) that exclude the selectant define the surface location to which the selectant binds.

By this embodiment, the selectants define a “chemical space” for the design of chemical libraries to search for drug leads that perform as the selectant. The selectants are particularly useful as chemical tools in high-throughput screening assays to identify chemical entities that compete with the selectant for the same target surface locus, identifying the chemical entity as a drug lead.

IV. Enhanced Combinatorial Peptide Display Library

A further embodiment of this invention provides enhanced combinatorial peptide-display libraries in which the displayed peptide is ribosome-associated, and the RNA encoding the peptide is retained as a ribosome-associated RNA. This allows for collection of positive clones by panning, with the encoding RNA recoverable as well for cloning, and sequencing.

In this embodiment of peptide display technology, bacteriophage biology is not obligatory. The instant approach exploits a feature of the prokaryote translation system, i.e., the ability of an RNA molecule lacking a termination codon to lock a ribosome into a quasi-stable “ternary complex” consisting of the peptide-ribosome-mRNA. This complex can be captured by a variety of methods including panning protocols and the encoding RNA can be recovered and cloned, providing a connection between associating peptide and the mRNA sequence encoding it. This approach increases the potential chemical diversity of the display library and accommodates novel scaffolds not readily adaptable to phage display. An additional advantage is the elimination of any requirement for the peptide fold to be permissive of phage viability.

When the prokaryote-translation apparatus is translating an mRNA that abruptly terminates without a stop codon the mRNA/ribosome/nascent polypeptide chain complex becomes locked into a quasi-stable complex we will refer to as a Frozen Translation Unit (FTU). In vivo, this complex is conveniently recovered by a process that employs two bacterial components that work together, small protein B (spB) and transfer-messenger RNA (tmRNA). The recovery process is initiated by tmRNA and spB binding to the vacant tRNA binding site on the FTU. Once the spB/tmRNA binds to the ribosome in the vacant “A” tRNA binding site the nascent polypeptide chain is transferred to tmRNA. The synthesis of the protein molecule is completed using a quasi-mRNA sequence that is part of the tmRNA structure. To capture FTUs from an in vitro translation system spB and tmRNA are removed from the in vitro translation system.

The mRNA family encoding for the combinatorial peptide array is generated by any convenient methods of in vitro mutagenesis. Useful vectors and templates have an RNA pol start transcription site upstream of the multi cloning site. A polypeptide template that has been cloned into the multicloning site usefully has a flexible carboxyl terminus capable of presenting the display peptide at a distance from the ribosome, what ever constant domains are included, and a flexible linkage between the constant domain and the variegated peptide (if necessary), with the variegated occupying the amino terminus of the displayed polypeptide.

V. Modulation of Protein-Protein Interactions

The process of this invention yet further includes isolation and identification of reagents that block specific protein-protein interactions (PPI_{br}). In particular such protein-protein interactions occur as the result of one protein molecule bridging two or more other protein molecules. In some embodiments of this process having known atomic coordinates for the formed multi-protein complex is advantageous. The goals of the process, however, are also achieved with a less rigorous structural foreknowledge. The PPI_{br} discovered by this process are usefully assembled into structures. By way of example, with epo there are 2 identical EPOR molecules that approach close enough such that their intracellular domains interact sufficiently to allow signal propagation. Thus, a structure is determined by the process of this invention that associates with the face of the c-terminal FNIII domain that serves as a steric block to the approach of the second EPOR. In “assembly,” two of these structures are joined with their FNIII domain contact surfaces facing in opposite direction. Such a molecule binds to one EPOR and is positioned to “compel” a second EPOR molecule to associate into a bi-receptor complex that positions the two intracellular domains close enough together to facilitate signal propagation. of the multi-protein complex in the absence of the bridging protein molecule. Without being bound by any particular theory its is believed that the receptors are conveniently viewed as “transducing elements”, as they have structures in both the extracellular and intracellular compartments, and they communicate (or transduce) the signal, represented as a constituent in the extracellular space (the hormone epo) to the intracellular environment (the intracellular domains that propagate the signal). One utility of this approach is generation of orally available therapeutic antagonist and agonist molecules. Particular utility for such molecules in cancer treatment and hormone replacement therapy. In hormone replacement-therapy it is therapeutic to establish hormonal sufficiency in a state where the hormone is being under produced. In such cases treatment with an agonist is useful. For example a peptide that activates the receptor in the same manner as the hormone does (treating diabetes with insulin, kidney failure with EPO, post-menopause with estrogen, castration with testosterone, etc). For cancer chemotherapy, in instances where there is an excessive hormonal stimulus, such as from a hormonal overproduction or expression of a receptor fueling cell growth it is desirable to block the action with an antagonist (IGF-I in some prostate and breast cancer, EGF in some solid tumors, testosterone in prostate cancer, growth hormone in acromegaly).

### EXAMPLE

### Selective PPI_{br}

A PPI_{br} [protein-protein interaction blocking reagent] is designed to block the formation of the activated complex consisting of two erythropoietin receptors bridged by one protein molecule (here erythropoietin), but not, in this example, block the interaction of one erythropoietin receptor with an erythropoietin molecule. This PPI_{br}, blocks the accretion of the second erythropoietin receptor to the pre-formed erythropoietin receptor-erythropoietin complex.

Information, materials, and methods useful in PPI_{br} preparation include:
The extracellular domain of the human erythropoietin receptor
   Modifications described in Syed et al (1998) Nature 395:515 for expression in eukaryote expression systems (CHO or Pichia pastoris) is described in Table 1 (the product will be referred to as EPObp) (For the quantities required for the described exercise, the CHO, 293 EBNA, or other cell culture systems will be adequate or are adjusted in a manner known by one of ordinary skill in the art.).
   An additional alteration to the EPObp is added at the amino terminus to facilitate immobilization of the target EPObp in streptavidin coated microplates. By “alteration” it is meant that: any amino- or carboxyl-terminal change which facilitates immobilization or affixation is usefully (and optionally) included. Alternatively no alteration need be made. Reference is made to optional use of an antibody to the amino-terminal FNIII domain that doesn't interfere with EPO binding.
      The sequence (G L N D I F E A Q K I E W H E) is added to the amino-terminus of the EPObp. Without being bound by any particular theory it is believed to allow the in vitro enzymatic biotinylation of the EPObp in accordance with the recommendations of Avidity (Denver, Colo.).
   A panel of EPObp charge-to-alanine mutants is generated. In one embodiment EPObp charge-to-alanine mutants comprise amino acids on the carboxyl-terminal FNIII domain, with charged side chains that project into the space between the two opposing EPORs in the ternary complex (EPOR-EPO-EPOR). (R=arginine, D=aspartic acid, E=glutamic acid, A=alanine) (see Table 2)
      R130A
      D133A
      E134A
      R141A
      R171A
      E173A
      E176A
      R178A
      E180A
      R187A
   Human erythropoietin (EPO) (unlabled and labeled with ¹²⁵I) will be used to establish proper folding of the EPObp constructs by assessing EPO binding isotherms in classical competition assays.
Bacteriophage peptide display libraries (libraries)
Conjugated antibodies directed against non-variegated bacteriophage coat proteins for use in detecting bound bacteriophage using a microplate reader.
   Process Description:
Initial panning step
   Pre-adsorb the library with the immobilization matrix minus the target, i.e., streptavidin coated wells without b-EPObp to remove library components with affinity for binding the matrix, in this example.
   Adsorb the pre-adsorbed library with immobilized b-EPObp
      Sequential harvesting
         Remove the supernatant and retain as devoid of binders (0)
         Wash once and retain as containing the weakest binders (1)
         Wash a second time and retain as containing weak binders (2)
         Wash a third time and retain as containing poor binders (3)
         Wash a forth time and retain as containing modest binders (4)
         Wash a fifth time and retain as containing moderate binders (5)
         Elute the remaining material and retain as containing strong binders (6)
Assessment of Strong Binders
   Clone the strong binders using accepted practices, and assess 96 clones for insert size and insert sequence.
   Choose those clones containing inserts with non-identical sequences for primary selection
   Prepare microplates in which columns 1 and 12 contain b-EPObp, and in which columns 2-11 contain the individual charge to alanine mutants, described above, i.e., 2=R130A, 3=D133A, 4=E134A, 5=R141A, 6=R171A, 7=E173A, 8=E176A, 9=R178A, 10=E180A, and 11=R187A.
   Each clone to be evaluated is incubated with an entire row of microplates prepared as described in the preceding step, i.e., the native b-EPObps in wells 1 and 12, and each of the charge to alanine mutants in wells 2-11.
      Following incubation each well is washed.
      Each well is than incubated with the anti-bacteriophage conjugated antibody.
      Un-bound antibody is removed by washing.
      Each well is incubated with the chromogenic substrate and the amount of bound bacteriophage is estimated by the color intensity assessed by the mictoplate reader.
   Assessment of strong binders
      Those bacteriophage that bind equally to each well of the row are declared to bind to b-EPObp surfaces distinct from those defined by the locations of the charge to alanine mutations, and probably bind to the EPO binding site.
      Those bacteriophage that bind to wells 1 and 12, as well as most of the other wells, but not all of the other wells, are declared to bind to a region of the b-EPObp defined by the specific charge to alanine mutants to which the bacteriophage fails to bind. For example, if the bacteriophage binds to all wells except wells 8 and 9, then the bacteriophage likely associated with the EPOR near E178 and R178.

All of the bacteriophage that are identified by the above screening protocol as associating with the circumscribed protein surface are optimized for affinity by affinity maturation, synthesized as peptides and reassessed for binding. Those peptides that behave as the phage guide the design of chemical libraries, using computational chemistry. The chemical libraries are then screened for target binding by displacement of the conjugates, cognate peptide to discover drug leads.

TABLE 1
EPOR swiss prot accession #p19235

| Key | From | To | Length | Description |
|---|---|---|---|---|
| SIGNAL | 1 | 24 | 24 | |
| CHAIN | 25 | 508 | 484 | ERYTHROPOIETIN RECEPTOR. |
| DOMAIN | 25 | 250 | 226 | EXTRACELLULAR (POTENTIAL). |
| TRANSMEM | 251 | 273 | 23 | POTENTIAL. |
| DOMAIN | 274 | 508 | 235 | CYTOPLASMIC (POTENTIAL). |
| DOMAIN | 148 | 213 | 66 | FIBRONECTIN TYPE-III. |
| DISULFID | 52 | 62 | | |
| DISULFID | 91 | 107 | | |
| CARBOHYD | 76 | 76 | | N-LINKED (GLCNAC . . . ) (POTENTIAL) |

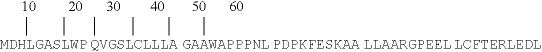

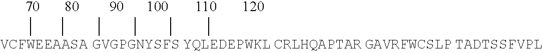

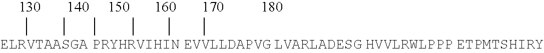

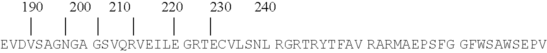

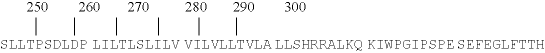

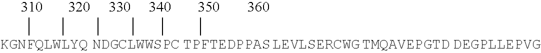

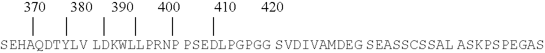

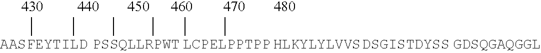

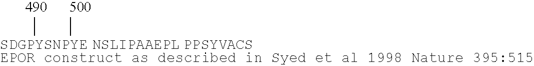

A25 redifined as aa#1
specific mutations shown in red: N52Q, N164Q, and A211E
The ala, shown in orange was replaced by arg-glu-phe (REF)

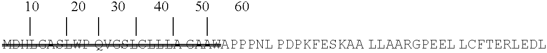

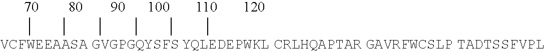

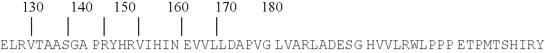

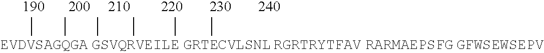

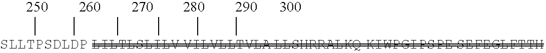

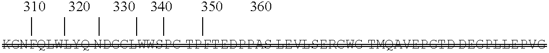

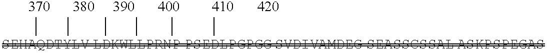

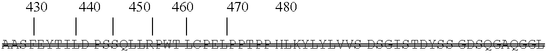

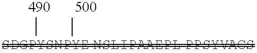

TABLE 2
Charge to alanine EPObp mutants//those amino acids depicted in red
will be individually changed to alanine

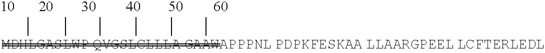

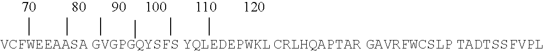

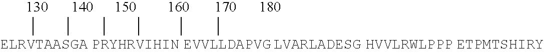

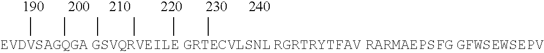

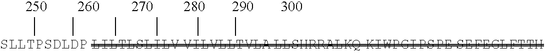

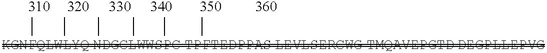

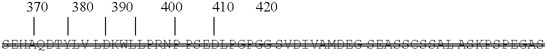

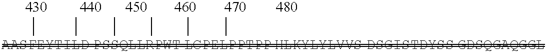

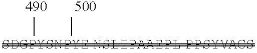

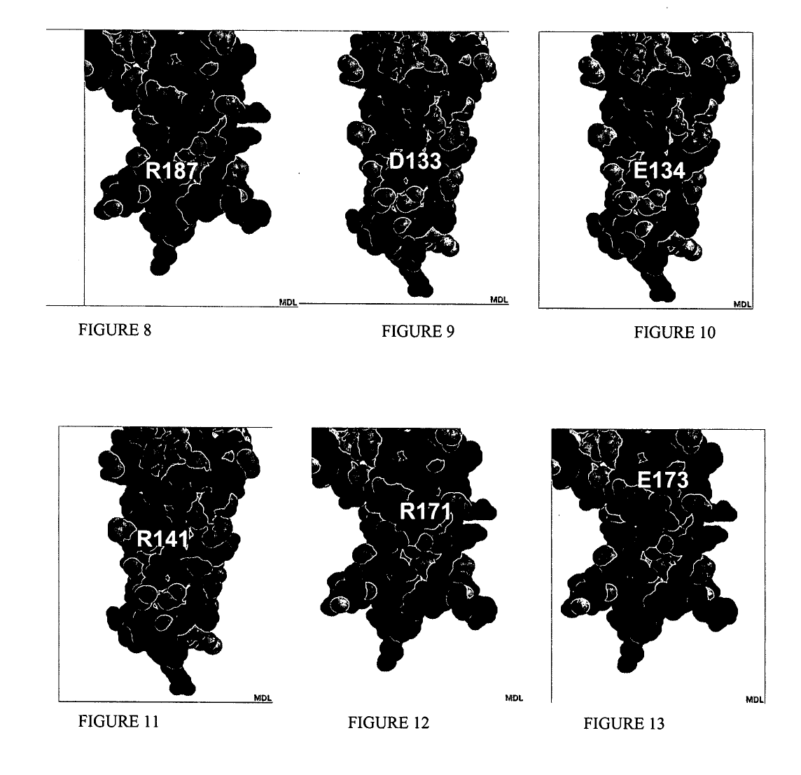

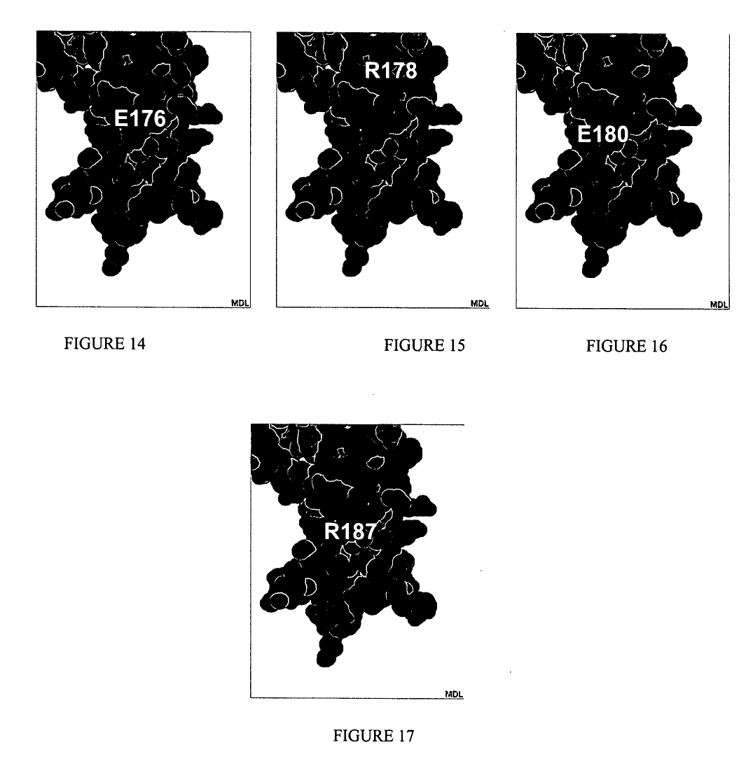

FIGS. 8 through 17 depict the amino acid side chains to be mutated to the alanine methyl group in the panel of mutants used to identify peptides from a sub-library selected by an initial panning procedure associated with a targeted EOPbp sub-domain. Numbers on the figures are counted from the amino terminus. The orientation of the EPObp seen in R130 (FIG. 8), D133 (FIG. 9), E134 (FIG. 10), and R141 (FIG. 11), R171 (FIG. 12), E172 (FIG. 13), E176 (FIG. 14), R178 (FIG. 15), E180 (FIG. 16) and R187 (FIG. 17) are of the EPObp in rightward rotational views.

Construction of Phage Display Libraries and Modification of Target Proteins

Library Construction

Preparation of Competent Cells

1. Inoculate 10 ml of LB/Tc medium with a single colony of E. coli WK6λmutS (Mobitec) and incubate at 37° C. and 180 rpm overnight.
2. The next day, inoculate 1000 ml of LB/Tc medium (2×500 ml Erlenmeyer flasks) at 1% with the overnight grown culture and incubate again at same conditions until an optical density of OD₆₀₀=0.6 has been reached.
3. Transfer 250 ml aliquots of the culture into centrifuge tubes (GS3), chill them on ice and centrifuge for 15 minutes at 6,000 rpm and 4° C. (Sorvall RC5C centrifuge; GS3 rotor).
4. Re-suspend each pellet in 250 ml of ice-cold H₂O and repeat the centrifugation step.
5. Re-suspend each pellet in 125 ml of ice-cold H₂O, pour together two aliquots and centrifuge again.
6. Re-suspend each pellet in 10 ml of ice-cold glycerol (10%) collect both aliquots in a GSA centrifuge tube and centrifuge for 15 minutes at 8,000 rpm.
7. Finally Re-suspend the bacterial pellet in 1 ml of glycerol (10%).
8. Fill 50 μl aliquots in precooled, sterile Eppendorf (Ep) reaction tubes, freeze immediately in liquid nitrogen and store at −70° C. until the transformation by electroporation.
   Helper Phage: M13K07Phage Stocks
1. The preparation of M13K07 helper phages should be started from a single fresh phage plaque. Therefore, inoculate 20 ml of LB medium with a single colony of E. coli WK6 cells and incubate over night at 180 rpm and 37° C.
2. Use 200 μl of this culture to inoculate 20 ml LB medium and incubate at the same conditions until the culture reaches the logarithmic growth phase (2-3 hours; OD₆₀₀=0.5).
3. Mix 1 μl of a M13K07 phage stock solution (Pharmacia) and 0.5 ml of logarithmic growing WK6 cells with 3 ml of molten LB top agar (about 40° C.) and pour the mixture onto a LB agar plate and incubate over night at 37° C.
4. The next day, use a sterile disposable Pasteur pipette to pick a single, well separated phage plaque and inoculate 20 ml of LB (2×)/Km medium (100 ml Erlenmeyer flask).
5. Incubate over day (6-8 hours) at 37° C. on a shaker at 180 rpm.
6. Inoculate 2×500 ml LB (2×)/Km medium with 10 ml preculture and incubate overnight (37° C., 180 rpm).
7. The next day, centrifuge four 250 ml aliquots for 15 minutes at 8,000 rpm and 4° C. (GS3 rotor, Sorvall RC5C).
8. Transfer the supernatant into centrifuge bottles and centrifuge again.
9. Transfer the supernatant again, add 0.15 vol of PEG/NaCl solution, mix and incubate on ice for at least 2 hours.
10. Centrifuge for 40 minutes at 8,000 rpm (GS3 rotor), decant the supernatant, repeat the centrifugation for 1 minute at 4,000 rpm and remove last traces of supernatant using a pipet.
11. Re-suspend each PEG-pellet in 2.5 ml PBS solution and collect the Re-suspended phages in one SS34 centrifuge bottle.
12. To clear the suspension centrifuge again for 10 minutes at 12,000 rpm (SS34 rotor).
13. Recover the supernatant (pipet), add NaN₃ to a final concentration of 0.02% and store the phages at 4° C.
   Transformation of pEVO_p7.Vec (the method of preparation and sequence of pEVO_{—}7.Vec is found below in Evolution of pSCAN8 to pEVO.vec.doc, Step 4b IN pEVO_Fyn.vec.doc and Step 4b Out pEVO_{—}7.vec.doc) into competent CJ236 cells. It is important to know that the glycine linker (flexible linker) connecting the polyprolyl domain (known peptide region) that binds to the Fyn SH3 domain (known target region) and the cysteine flanked combinatorial sequence (inquiry peptide) can also be linkers of other lengths and flexibility.
1. Gently thaw the competent CJ 236 cells on ice. For each control and sample reaction to be transformed, aliquot 50 μl of the competent cells to a prechilled 15 ml conical tube.
2. Add 0.5 μg of pEVO_p7.Vec to the competent cells. Swirl the transformation reactions gently to mix and incubate the reactions on ice for 30 minutes.
3. Heat pulse the transformation reactions for 3 min 37° C. and then place the reactions on ice for 2 minutes.
4. Add 0.8 ml of SOC medium and incubate the transformation reactions at 37° C. for 1.5 hours with shaking at 200-250 rpm.
5. Spin the tube at 3,000 rpm for 5 min and re-suspend cells in 200 μl LB medium.
6. Plate the cells on agar plates containing Chloramphenicol and Carbenicillin.
7. Incubate the transformation plates at 37° C. overnight.
   Preparation of dU-ssDNA Template
1. From the plate, pick a single colony of E. Coli CJ236 harboring pEVO_p7.Vec into 10 ml of 2 YT media (10 g bacto-yeast extract, 16 g bacto-tryptone, 5 g NaCl per Liter of H₂O) supplemented with 100 μg/ml carbenicillin to select for pEVO_p7.Vec, and 17 μg/ml chloramphenicol to maintain the CJ236 F′ episome.
2. Shake at 200 rpm and 37° C. for 6-8 hours.
3. Add M13K07 helper phage to a final concentration of 10¹⁰ phage/ml and shake at 200 rpm and 37° C. for 15 min.
4. Transfer the culture to 300 ml of 2YT/carb/uridine media. Shake overnight at 200 rpm and 37° C.
5. Centrifuge for 10 min at 15000 rpm and 4° C. in a Sorvall SS-34 rotor (27,000 g).
6. Transfer the supernatant to a new tube containing ⅕ volume of PEG/NaCl and incubate for 5 min at room temperature.
7. Centrifuge 10 min at 10000 rpm and 4° C. in an SS-34 rotor (12,000 g).
8. Decant the supernatant. Centrifuge briefly at 4,000 rpm (2000 g) and aspirate the remaining supernatant.
9. Re-suspend the phage pellet in 0.5 ml of PBS. Centrifuge for 5 min at 15,000 rpm and 4° C. in an SS-34 rotor to pellet insoluble matter.
10. Transfer the supernatant to a 1.5 ml micro-centrifuge tube.
11. Add 7.0 ml of Buffer MP, mix and incubate at room temperature for at least 2 min.
12. Apply the sample to a QIAprep spin column (Qiagen) in a 2 ml micro-centrifuge tube. Use one QIAprep column for every 50 ml of overnight culture.
13. Centrifuge for 15 s at 8,000 rpm in a micro-centrifuge. Discard the flow-through. The phage particles remain bound to the column matrix.
14. Add 0.7 ml Buffer MLB to the column. Centrifuge for 15 s at 8,000 rpm. Discard the flow-through.
15. Add another 0.7 ml Buffer MLB. Incubate at room temperature for at least 1 min.
16. Centrifuge at 8,000 rpm for 15 s. Discard the flow-through.
17. The DNA is separated from the protein coat and remains adsorbed to the matrix.
18. Add 0.7 ml Wash Buffer PE. Centrifuge at 8,000 rpm for 15 s. Discard the flow-through.
19. Repeat step 12 to remove residual proteins and salt.
20. Centrifuge at 8,000 rpm for 30 s. Transfer the column to a fresh 1.5 ml micro-centrifuge tube.
21. Add 100 μl of Buffer EB (10 mM Tris-HCl, pH 8.0) to the center of the column membrane. Incubate at room temperature for 10 min and centrifuge for 30 s at 8,000 rpm. The eluant contains the purified dU-ssDNA.
22. Determine the DNA concentration by measuring absorbance at 260 nm (A=1.0 for 33 ng/μl of ssDNA).
   Phosphorylation of the Mutagenic Oligonucleotide (EvoVec6mer R)
1. Combine 20 μg of the oligonucleotide with 20 μl 10× Ligation Buffer (Roche). Add water to a total volume of 200 μl.
2. Add 50 units of T4 Polynucleotide kinase and incubate at 37° C. for 1 h
   For the 6mer Library, we Used a Commercially Synthesized Oligonucleotude

Evo Vec6mer R, N=any nucleotide and M=A or C.

AGCCACCGCCGCCGGCGGTACCGCAMNNMNNMNNMNNMNNMNNGCAACCG
GCGAGCTCGGCCTGCGCTACGGTAGCG

Annealing the Oligonucleotide to the Template
Note: The protocol below is described for one reaction. For a 6mer library (64×10⁶ clones), we need 10 such reactions.
1. Take 6 μg of the dU-ssDNA template and add oligonucleotide to give a template to oligonucleotide molar ratio of 1:10. Add 12.5 μl of 10×TM Buffer (0.5 M Tris, pH 7.5, 0.1 M MgCl₂) and add water to a total volume of 125 μl.
2. Incubate at 90° C. for 2 min, 50° C. for 3 min and 20° C. for 5 min
   Enzymatic Synthesis of Covalently Closed Circular (CCC) DNA
1. To the annealed oligonucleotide/template mixture, add 0.5 μl 100 mM ATP, 5 μl 25 mM dNTP, 0.7 μl 1.25 M dTT, 30 units T4 DNA Ligase, 30 Units T7 DNA Polymerase, 0.5 μl BSA (NEB), 0.5 μl ssBP (1 μg/μl; Stratagene)
2. Incubate overnight at 20° C.
3. Affinity purify and desalt the DNA using the QIAquick DNA purification kit (Qiagen).
4. Add 1.0 ml of buffer QG and mix.
5. Apply the sample to two QIAquick spin columns placed in 2 ml micro-centriftige tubes.
6. Centrifuge at 13,000 rpm for 1 min in a micro-centrifuge and discard the flow-through.
7. Add 750 μl of buffer PE to each column.
8. Centrifuge at 13000 rpm for 1 min and discard the flow-through and centrifuge at 13000 rpm for 1 min.
9. Place the column in a new 1.5 ml micro-centrifuge tube.
10. Add 35 μl of ultrapure water to the center of the membrane and incubate at room temperature for 1 min.
11. Centrifuge at 13,000 rpm for 1 min to elute the DNA.
12. The DNA can be used immediately for E. coli electroporation, or it can be stored frozen for later use.
   Electroporation of Competent Cells
1. Place frozen aliquots of competent E. coli WK6λmutS cells on ice and let them thaw.
2. To each aliquot add 35 μl of purified DNA and incubate on ice for 10 minute.
3. Fill the suspension in a pre-chilled electroporation cuvette, place the cuvette in the electroporation sled and give a pulse at a voltage of 1.8 kV, a capacity of 25 μF and a resistance of 200Ω (Gene Pulser and Puls Controller, Bio-Rad).
4. Immediately add 1 ml of LB medium, mix and transfer the suspension in a 15 ml conical tube.
5. Incubate for 1 hour at 37° C. and plate on LB agar containing ampicillin (100 μg/ml) and tetracycline (20 μg/ml).
6. Incubate overnight at 37° C.
7. In the same way carry out a transformation with and without pEVO_p7.vec DNA as a control and plate out on LB/Tc and LB/Amp/Tc plates.
8. Also plate serially diluted aliquots of transformed cells in order to calculate the size of the final library.

9. As a test, the individual clones of the library can be sequenced using the following primer: Lib

Seq: GCCCTGAAGAAGGGCAGC

Packaging of Phagemids from Cells
1. Re-suspend the complete lawns of the E. coli cells in 20 ml of LB/Amp/Tc medium and use 2 ml for inoculation of 50 ml LB/Amp/Tc medium (250 ml Erlenmeyer flask).
1. Incubate at 180 rpm and 37° C. for 1 hour, add 100 μg of M13K07 stock solution (10¹¹-10¹² cfu/ml) and incubate for 15 minutes at 37° C. without shaking.
2. Allow the culture to shake at 37° C. for 45 min, then add Kanamycin (final concentration of 50 μg/ml) and continue the incubation at 37° C. @ 180 rpm overnight.
3. The next day, centrifuge for 15 minutes at 8,000 rpm and 4° C. (GS3 rotor, Sorvall RC5C).
4. Transfer the supernatant into a new centrifuge bottle and centrifuge again.
5. Transfer the supernatant again, add 0.15 vol of PEG/NaCl solution, mix and incubate on ice for at least 2 hours.
6. Centrifuge for 40 minutes at 8,000 rpm (GS3 rotor)
7. Decant the supernatant, repeat the centrifugation for 1 minute at 4,000 rpm and remove last traces of supernatant using a pipet.
8. Re-suspend each PEG-pellet in 1.0 ml PBS solution and collect the re-suspended phages in one SS34 centrifuge bottle
9. To clear the suspension, centrifuge again for 10 minutes at 12,000 rpm (SS34 rotor).
10. Recover the supernatant (pipet), add NaN₃ to a final concentration of 0.02% and store the library at 4° C.
   Determination of Phagemid (Library) Titer (Colony Forming Units [CFU] Assay)
1. Inoculate 20 ml of LB/Tc (20 μg/ml) medium with 200 μl of an E. coli WK6λmutS overnight culture (37° C.; LB/Tc) and incubate at 37° C. and 180 rpm for 2 to 3 hours (OD₆₀₀=0.5)
2. Fill 12 wells of a sterile 96-well culture dish with 90 μl of autoclaved water and prepare dilution series by transferring 10 μl aliquots of the library stock (dilutions 10⁻¹ to 10⁻¹²).
3. Add 100 μl of logarithmic growing cells, mix and incubate for 30 min at 37° C.
4. Spot 20 μl portions of each well on LB/Amp/Tc agar plates and incubate over night at 37° C.
5. As a control use 20 μl of non-infected log-phase cells spotted on LB/Amp/Tc and LB/Tc agar plates.
6. Count the number of colonies on the next day and determine the titer.
   Panning
   Protein Targets
WT SCCE is the Stratum Corneum Chymotryptic Enzyme subdloned in the pIE vector and adapted with a carboxy-terminal polyglycine linker and polyhistidine sequence to facilitate purification and immobilization (see WT SCCE preparation below). The adaptations were performed using a QuickChange mutagenesis kit. The sequence is listed in document SCCE His6 pIE.doc.
Fyn SCCE is the Stratum Corneum Chymotryptic Enzyme adapted with a polyglycine liker, the Fyn SH3 domain and additional polyglycine linker and a polyhistidine sequence (see Fyn SCCE preparation below). The final sequence is listed in document SCCE FYN His6 pIE.doc. The modifications to create Fyn SCCE were performed using WT SCCE as a template using a QuickChange mutagenesis kit.
The expression and purification of WT SCCE and Fyn SCCE are presented in document SCCE production.doc
   Pre-Adsorption of Library with Matrix to Remove Non-Specific Binding
1. Take 25 μl Ni-NTA Magnetic Agarose Beads (Qiagen) in a 1.5 ml tube (siliconized), labeled pre-adsorption library.
2. Place in Magnet and remove Sup.
3. Wash 2 times (2 min each time) with 500 μl Ni-NTA Binding Buffer (50 mM NaH₂PO₄ pH8.0, 300 mM NaCl, 10 mM Imidazole) to equilibrate beads to Immidizole.
4. Add 200 μl TBS-T 0.1% BSA+200 μl (10¹² phage) of 6mer library to tube labeled pre-adsorption library (to remove phage that stick non-specifically to the beads).
5. Incubate @ room temperature for 1 h w/rotation.
   Coating Matrix with Target Protein
6. Take 25 μl Ni-NTA Magnetic Agarose Beads (Qiagen) in 2 separate 1.5 ml Ep tubes (siliconized), labeled SSCE-WT and SCCE-FYN.
7. Place in Magnet and remove Sup.
8. Wash 2 times (2 min each time) with (500 μl) Ni-NTA Binding Buffer (50 mM NaH₂PO₄ pH8.0, 300 mM NaCl, 10 mM Imidazole) to equilibrate beads to Immidizole.
9. Add 250 μl Ni-NTA wash (20 mM Imidazole)+250 μl TBS-T 0.1% BSA+protein (1.0 μg) SCCE-WT and SCCE-FYN to the tubes labeled SSCE-WT and SCCE-FYN respectively.
10. Incubate all tubes @ R.T. for 1 h w/rotation
   Panning Against SCCE-WT
11. Remove Sup from SSCE-WT tube using magnet and wash beads 2 times with TBS-T 0.1% BSA
12. Transfer sup from tube labeled pre-adsorption library to tube labeled SSCE-WT.
13. Incubate for 1 h with rotation @ R.T. (now, the library is incubating with beads coated with SCCE-WT)
14. Remove Sup and save for step # 21
15. Wash beads 4 times (5 min each time) with TBS-T 0.1% BSA (500 μl each time)
16. Elute with 100 μl Elution Buffer Glycine pH 2.0 (10 min with rotation @ R.T.)
17. Immediately after elution, add 12.5 μl Neutralization Buffer 1 M Tris pH 9.0
18. Add sodium azide to a final concentration of 0.02%
19. Save elution and label it as “SCCE-WT Round N” and store @ 4° C.
   Panning Against SCCE-FYN
20. Remove Sup from SCCE-FYN tube using magnet and wash beads 2 times with TBS-T 0.1% BSA
21. Take sup from the SSCE-WT tube and add to the SSCE-FYN tube.
22. Incubate for 1 h with rotation @ R.T (now, the library is incubating with beads coated with SCCE-FYN)
23. Remove Sup
24. Wash beads 4 times with 500 μl TBS-T 0.1% BSA (5 min each time)
25. Elute with 100 μl Elution Buffer Glycine pH 2.0 (10 min with rotation @ R.T.)
26. Immediately after elution, add 12.5 μl Neutralization Buffer 1 M Tris pH 9.0
27. Add sodium azide to a final concentration of 0.02%
28. Save elution and label as “SCCE-FYN Round N” and store @ 4° C.
   Note: At the end of the first round of panning, there will be two populations of phage, one from panning against SCCE-WT and another from panning against SCCE-FYN. For each of these populations of phage, we determine the titer, re-infect E. Coli and package the phagemids from the re-infected cells as described below. For the subsequent rounds of panning, the procedure remains exactly the same except that the output phage from Round “N” is used as input phage for Round “N+1”. Also, the phage obtained from panning against SCCE-WT in a given round is used as input to pan against SCCE-WT for the next round. Similarly, the phage obtained from panning against SCCE-FYN in a given round is used as input to pan against SCCE-FYN for the next round.
   Determination of Phagemid Titers (Colony Forming Units [CFU] Assay)
1. Inoculate 20 ml of LB/Tc (20 μg/ml) medium with 200 μl of an E. coli WK6?mutS overnight culture (37° C.; LB/Tc) and incubate at 37° C. and 180 rpm for 2 to 3 hours (O.D.₆₀₀=0.5)
2. For each phagemid probe fill ten wells of a sterile 96-well culture dish with 90 μl of autoclaved water and prepare dilution series by transferring 10 μl aliquots (dilutions 10⁻¹ to 10⁻¹⁰).
3. Add 100 μl of logarithmic growing cells, mix and incubate for 30 min at 37° C.
4. Spot 20 μl portions of each well on LB/Amp/Tc agar plates and incubate over night at 37° C.
5. As a control use 20 μl of non-infected log-phase cells spotted on LB/Amp/Tc and LB/Tc agar plates.
6. Count the number of colonies on the next day to determine the titer from the output of panning
   Re-Infection of E. coli cells
1. Mix the eluted phages and 20 ml of E. coli WK6λmutS log-phase cells (37° C.; LB/Tc-culture) and incubate for 30 min at 37° C.
2. Collect the cells by centrifugation (5 minutes, 8,000 rpm, SS34 rotor) and Re-suspend the pellet in 400 μl of LB/Amp (250 μg/ml)/Tc (20 μg/ml) medium.
3. Plate 200 μl aliquots onto LB/Amp/Tc agar plates and incubate them overnight at 37° C.
   Packaging of Phagemids from Re-Infected Cells
1. Re-suspend the complete lawn of the Re-infected E. coli cells in 20 ml of LB/Amp/Tc medium and use 2 ml for inoculation of 50 ml LB/Amp/Tc medium (250 ml Erlenmeyer flask).
2. Incubate at 180 rpm and 37° C. for 1 hour, add 100 μl of M13K07 stock solution (10¹¹-10¹² cfu/ml) and incubate for 15 minutes at 37° C. without shaking.
3. Allow the culture to shake at 37° C. for 45 min, then add Kanamycin (final concentration of 50 μg/ml) and continue the incubation at 37° C. @180 rpm overnight.
4. The next day, centrifuge for 15 minutes at 8,000 rpm and 4° C. (GS3 rotor, Sorvall RC5C).
5. Transfer the supernatant into a new centrifuge bottle and centrifuge again.
6. Transfer the supernatant again, add 0.15 vol of PEG/NaCl solution, mix and incubate on ice for at least 2 hours.
7. Centrifuge for 40 minutes at 8,000 rpm (GS3 rotor)
8. Decant the supernatant, repeat the centrifugation for 1 minute at 4000 rpm and remove last traces of supernatant using a pipet.
9. Re-suspend each PEG-pellet in 1.0 ml PBS solution and collect the Re-suspended phages in one SS34 centrifuge bottle
10. To clear the suspension centrifuge again for 10 minutes at 12,000 rpm (SS34 rotor).
11. Recover the supernatant (pipet), add NaN₃ to a final concentration of 0.02% and store the phages at 4° C. for the next round of panning.

Following four rounds of panning against WT SCCE and Fyn SCCE, a subset of randomly selected clones were sequenced using the Lib Seq sequencing primer listed below.

Lib Seq: GCCCTGAAGAAGGGCAGC

The sequences obtained from the WT SCCE panning are listed in document 6mer R4 SCCE WT sequences, below. The sequences obtained from the Fyn SCCE panning are listed in the document 6mer R4 SCCE Fyn sequences, below.
Evolution of pSCAN8 to pEVO.vec 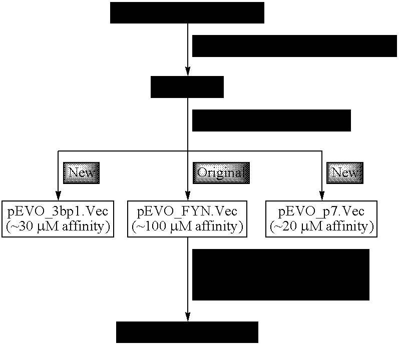
Step 1: pSKAN8 to pEVO.Vec
Start with: Step 1 IN pSKAN8
End With Step 1 Out pEVO.Vec
Introduction of a Flex-HVD-Flex and Removal of hPstI from pSKAN8

Primers Used:

pSKAN8 F:
L I H E E G E
GGTACCGCCGGCGGCGGTGGCTCGGGCGGAGGCTCTGGGGGGGGCTTAAT
TCATGAAGAAGGTGAA

The highlight (Arial type face) shows the leading portion of the forward primer that lays down on the template.

pSKAN8 R:
A Q A V T A
GCAAACCGGGTCGTAGATCTTAGTGCAACCGGCGAGCTCGGCCTGCGCTA
CGGTAGCG

The highlight (Tahoma type face) shows the leading portion of the reverse primer that lays down on the template.
Method: QuikChange® Site-Directed Mutagenesis Kit from Stratagene.
Phosohorylation of Primers:
6.25 μl of pSKAN8 F (1 μg/μl)
6.25 μl of pSKAN8 R (1 μg/μl)
5 μl 10×PNK (Polynucleotide Kinase) Buffer from NEB
1 μl ATP
1 μl T4 PNK (NEB)
5 μl of 10× reaction buffer
X μl (250 ng) of dsDNA template
X μl (125 ng) of oligonucleotide pSKAN8 F (phosohorylated)
X μl (125 ng) of oligonucleotide pSKAN8 R (phosohorylated)
1 μl of dNTP mix
ddH2O to a final volume of 50 μl
95° C. for 5 min
Ice; microfuge
Then add 1 μl of PfuTurbo DNA polymerase (2.5 U/μl)

Step I: 95° C. 30 seconds

Step II: 95° C. 30 seconds
55° C. 1 minute
68° C. 1 minute/kb of plasmid length (11 min for pSKAN8)

Repest Step II 17 times

Step III: 68° C. for 10 min

Step IV: 4° C. pause

Amplification is checked by electrophoresis of 5 μl of the product on a 1% agarose gel. A band is visible at this stage.

Dpn I Digestion and Transformation.

Add 1 μl of the Dpn I restriction enzyme (10 U/μl) directly to each amplification reaction and incubate reaction at 37° C. for 1 hour to digest the parental (i.e., the nonmutated) supercoiled dsDNA.

Transformation of XL1-Blue Supercompetent Cells

1. Gently thaw the XL1-Blue supercompetent cells on ice. For each control and sample reaction to be transformed, aliquot 50 μl of the supercompetent cells to a prechilled 15 ml conical tube.

2. Transfer 10 μl of the Dpn I-treated DNA from each control and sample reaction to separate aliquots of the supercompetent cells. Swirl the transformation reactions gently to mix and incubate the reactions on ice for 30 minutes.

3. Heat pulse the transformation reactions for 3 min 37° C. and then place the reactions on ice for 2 minutes.

4. Add 0.8 ml of SOC medium and incubate the transformation reactions at 37° C. for 1.5 hours with shaking at 200-250 rpm.

5. Spin the tube at 3000 rpm for 5 min and re-suspend cells in 200 μl LB medium. Plate the cells on agar plates containing the ampicillin.

6. Incubate the transformation plates at 37° C. for >16 hours.

7. Next day, pick up a single colony, grow overnight in 3 ml LB medium.

8. Use QIAprep spin miniprep kit for plasmid purification.

9. The sequence was confirmed with the following sequencing primers:

1255: GGGATTTTGCTAAACAAC
2897: GGAGGTCTAGATAACGAGG

Step 2: pEVO.Vec to pEVO_FYN.Vec
Start with: Step 2 IN pEVO.Vec
End With Step 2 Out pEVO_FYN.Vec
Insertion of Fyn Binding Domain into pEVO.Vec

Primers Used:

pEVO_Fyn_F:
G G S G G G L I H E E G
GTTTGGGACTTATCCTCCCCCTCTCCCTCCCGGAGGCTCTGGGGGGGGCT
TAATTCATGAAGAAGGT

The highlight (Arial type face) shows the leading portion of the forward primer that lays down on the template.

pEVO_Fyn_R:
G S G G G G A T G C V P D Y I K T
CCGCCCCCTCCGCCACCGCCCGAGCCACCGCCGCCGGCGGTACCGCAAAC
CGGGTCGTAGATCTTAGTGC

The highlight (Tahoma type face) shows the leading portion of the reverse primer that lays down on the template.
Method: QuikChange® Site-Directed Mutagenesis Kit from Stratagene.

The sequence was confirmed with the following sequencing primer:

Lib_seq:
GCCCTGAAGAAGGGCAGC

Step 3: pEVO_FYN.Vec to pEVO_Secondary.Vec
Start with: Step 3 IN pEVO_FYN.Vec
End With Step 3 Out pEVO_Secondary.Vec
Removal of FYN Binding Domain and insertion of G2_NQDVD_G2 & Constant Domain

Primers Used:

```
pEvo_Secondary F
  C G T G G N Q D V D G G K L R S G
TGCGGTACCGGCGGCAACCAGGACGTCGACGGCGGGAAGCTTAGATCTGG
    S L I H E E G E F S E A R E D
ATCCTTAATTCATGAAGAAGGTGAATTCTCAGAAGCGCGCGAAGAT
pEvo_Secondary R
  D E R A E S F E G E E H I L S G S
ATCTTCGCGCGCTTCTGAGAATTCACCTTCTTCATGAATTAAGGATCCAG
    R L K G G D V D Q N G G T G C
ATCTAAGCTTCCCGCCGTCGACGTCCTGGTTGCCGCCGGTACCGCA
```

The two primers were commercially synthesized fragments that were phosphorylated, annealed, and then digested with KpnI. Then, they were used as an insert and ligated into the vector (pEvo_Fyn.Vec) digested with KpnI and EcoRV
Step 1 Preparation of Vector
pEVO_Fyn.Vec 10 μg (X μl)
10×NEB R.E. Buffer#2 10 μl
BSA 0.6 μl
R.E. KpnI 2.5 μl
R.E. EcoRV 2.5 μl
H₂O up to 60 μl
37° C. for 3 hours
Phenol Chloroform Extract
Purify digested vector by running on an agarose get and use QIAquick Gel Extraction Kit
Run aliquot of eluate (purified digested vector) for quantitation
Step II Preparation of Insert
Step IIa: Phosphorylation of Primers
1 μl of pEvo_Secondary F (10 μg/μl)
1 μl of pEvo_Secondary R (10 μg/μl)
2 μl 10×PNK (Polynucleotide Kinase) Buffer from NEB
0.2 μl ATP
1 μl T4 PNK (NEB)
H₂O up to 20 μl
Step IIb: Annealing of Primers
95° C. 5 min
Slow cool to room temperature
Add 4.8 μl 10×NEB R.E. Buffer#2 and 21.2 μl H₂O
Add R.E. KpnI 2 μl
37° C. for 3 hours
Phenol Chloroform Extract
Purify using QIAquick Nucleotide Removal Kit
Run aliquot of eluate (purified digested insert) for quantitation
Step III: Ligation of vector and insert
Vector: pEVO_Fyn digested w/KpnI & EcoRV

Insert: Annealed primers digested with KpnI

| | | 10× | | |
|---|---|---|---|---|
| Vector | Insert | Ligation | | |
| (fmol) | (fmol) | Buffer (μl) | H₂O (μl) | Ligase (μl) |
| 30 | 60 | 2.5 | Upto 25 | 0.5 |
| 30 | 150 | 2.5 | Upto 25 | 0.5 |
| 30 | 300 | 2.5 | Upto 25 | 0.5 |
| 30 | 0 | 2.5 | Upto 25 | 0.5 |

12° C. for 16 hours (overnight)
Step IV: Transformation of Ligation Product into Competent C 7118 cells
1. Gently thaw the competent C 7118 cells on ice. For each control and sample reaction to be transformed, aliquot 50 μl of the competent cells to a prechilled 15 ml conical tube.
2. Transfer 25 μl of each ligation product to separate aliquots of the competent cells. Swirl the transformation reactions gently to mix and incubate the reactions on ice for 30 minutes.
3. Heat pulse the transformation reactions for 3 min 37° C. and then place the reactions on ice for 2 minutes.
4. Add 0.8 ml of SOC medium and incubate the transformation reactions at 37° C. for 1.5 hours with shaking at 200-250 rpm.
5. Spin the tube at 3000 rpm for 5 min and re-suspend cells in 200 μl LB medium. Plate the cells on agar plates containing the ampicillin.
6. Incubate the transformation plates at 37° C. for >16 hours.
7. Next day, pick up a single colony, grow overnight in 3 ml LB medium.
8. Use QIAprep spin miniprep kit for plasmid purification.
9. The sequence was confirmed with the following sequencing primers:

The sequence was confirmed with the following sequencing primer:

Lib_seq:
GCCCTGAAGAAGGGCAGC

Step 4a: pEVO_FYN.Vec to pEVO_{—}3bp1.Vec (˜30 μM affinity)
Start with: Step 4a IN pEVO_FYN.Vec
End With Step 4a Out pEVO_{—}3 bp1.Vec
Swapping 100 μM Affinity Fyn Binding Domain with Another that has 30 μM Affinity

Primers Used:

3bp1 IN F
S G G G G G G P P P L P P G
TCGGGCGGTGGCGGAGGGGGC [symbol] CCTCCCCCTCTCCC
G
TCCCGGAGG

The highlight (Arial type face) shows the portion of the forward primer that lays down on the template.

3bp1 IN R
G G P P L P P P G G G G G G S
CCTCCGGGAGGGAGAGGGGGAGGCATAGTCGGAGCCCGGCCCCCTCCGCC
ACCGCCCGA

The highlight (Tahoma type face) shows the portion of the reverse primer that lays down on the template.
Method: QuikChange® Site-Directed Mutagenesis Kit from Stratagene.

The sequence was confirmed with the following sequencing primer:

Lib_seq:
GCCCTGAAGAAGGGCAGC

Step 4b: pEVO_FYN.Vec to pEVO_p7.Vec (˜20 μM Affinity)
Start with: Step 4b IN pEVO_FYN.Vec
End With Step 4b Out pEVO_p7.Vec
Swapping 100 μM Affinity Fyn Binding Domain with Another that has 20 μM Affinity

Primers Used:

p7 IN F
G G G G G G P P G G
CGGGTGGCGGAGGGGGCGGG [symbol] CCTCCCG
S G
GAGGCTCTGG

The highlight (Arial type face) shows the portion of the forward primer that lays down on the template.

p7 IN R
G S G G P P G G G G
CCAGAGCCTCCGGGAGG [symbol] CCCGCCCCC
G G
TCCGCCACCG

The highlight (Tahoma type face) shows the portion of the reverse primer that lays down on the template.
Method: QuikChange® Site-Directed Mutagenesis Kit from Stratagene.

The sequence was confirmed with the following sequencing primer:

| Lib_seq: | |
|---|---|
| GCCCTGAAGAAGGGCAGC | |
| Step 4b IN pEVO_Fyn.vec | |
| 1 | ACGCTCTTAA AATTAAGCCC TGAAGAAGGG CAGCATTCAA AGCAGAAGGC TTTGGGGTGT |
| | TGCGAGAATT TTAATTCGGG ACTTCTTCCC GTCGTAAGTT TCGTCTTCCG AAACCCCACA |
| | EcoRI |
| 61 | GTGATACGAA ACGAAGCATT GGAATTCTAC AACTTGCTTG GATTCCTACA AAGAAGCAGC |
| | CACTATGCTT TGCTTCGTAA CCTTAAGATG TTGAACGAAC CTAAGGATGT TTCTTCGTCG |
| | XbaI |
| | M K K • |
| 121 | AATTTTCAGT GTCAGAAGTC GACCAAGGAG GTCTAGATAA CGAGGGCAAA AAATGAAAAA |
| | TTAAAAGTCA CAGTCTTCAG CTGGTTCCTC CAGATCTATT GCTCCCGTTT TTTACTTTTT |
| | SacI |
| | • T A I A I A V A L A G F A T V A Q A E L • |
| 181 | GACAGCTATC GCGATTGCAG TGGCACTGGC TGGTTTCGCT ACCGTAGCGC AGGCCGAGCT |
| | CTGTCGATAG CGCTAACGTC ACCGTGACCG ACCAAAGCGA TGGCATCGCG TCCGGCTCGA |
| | SacI BglII KpnI AvaI |
| | • A G C T K I Y D P V C G T A G G G G S G • |
| 241 | CGCCGGTTGC ACTAAGATCT ACGACCCGGT TTGCGGTACC GCCGGCGGCG GTGGCTCGGG |
| | GCGGCCAACG TGATTCTAGA TGCTGGGCCA AACGCCATGG CGGCCGCCGC CACCGAGCCC |
| | • G G G G G G F G T Y P P P L P P G G S G • |
| 301 | CGGTGGCGGA GGGGGCGGGT TTGGGACTTA TCCTCCCCCT CTCCCTCCCG GAGGCTCTGG |
| | GCCACCGCCT CCCCCGCCCA AACCCTGAAT AGGAGGGGGA GAGGGAGGGC CTCCGAGACC |
| | EcoRI EcoRV |
| | • G G L I H E E G E F S E A R E D I R A E • |
| 361 | GGGGGGCTTA ATTCATGAAG AAGGTGAATT CTCAGAAGCG CGCGAAGATA TCAGAGCTGA |
| | CCCCCCGAAT TAAGTACTTC TTCCACTTAA GAGTCTTCGC GCGCTTCTAT AGTCTCGACT |
| | • T V E S C L A K S H T E N S F T N V W K • |
| 421 | AACTGTTGAA AGTTGTTTAG CAAAATCCCA TACAGAAAAT TCATTTACTA ACGTCTGGAA |
| | TTGACAACTT TCAACAAATC GTTTTAGGGT ATGTCTTTTA AGTAAATGAT TGCAGACCTT |
| | • D D K T L D R Y A N Y E G C L W N A T G • |
| 481 | AGACGACAAA ACTTTAGATC GTTACGCTAA CTATGAGGGC TGTCTGTGGA ATGCTACAGG |
| | TCTGCTGTTT TGAAATCTAG CAATGCGATT GATACTCCCG ACAGACACCT TACGATGTCC |
| | • V V V C T G D E T Q C Y G T W V P I G L • |
| 541 | CGTTGTAGTT TGTACTGGTG ACGAAACTCA GTGTTACGGT ACATGGGTTC CTATTGGGCT |
| | GCAACATCAA ACATGACCAC TGCTTTGAGT CACAATGCCA TGTACCCAAG GATAACCCGA |
| | • A I P E N E G G G S E G G G S E G G G S • |
| 601 | TGCTATCCCT GAAAATGAGG GTGGTGGCTC TGAGGGTGGC GGTTCTGAGG GTGGCGGTTC |
| | ACGATAGGGA CTTTTACTCC CACCACCGAG ACTCCCACCG CCAAGACTCC CACCGCCAAG |
| | • E G G G T K P P E Y G D T P I P G Y T Y • |
| 661 | TGAGGGTGGC GGTACTAAAC CTCCTGAGTA CGGTGATACA CCTATTCCGG GCTATACTTA |
| | ACTCCCACCG CCATGATTTG GAGGACTCAT GCCACTATGT GGATAAGGCC CGATATGAAT |
| | • I N P L D G T Y P P G T E Q N P A N P N • |
| 721 | TATCAACCCT CTCGACGGCA CTTATCCGCC TGGTACTGAG CAAAACCCCG CTAATCCTAA |
| | ATAGTTGGGA GAGCTGCCGT GAATAGGCGG ACCATGACTC GTTTTGGGGC GATTAGGATT |
| | • P S L E E S Q P L N T F M F Q N N R F R • |
| 781 | TCCTTCTCTT GAGGAGTCTC AGCCTCTTAA TACTTTCATG TTTCAGAATA ATAGGTTCCG |
| | AGGAAGAGAA CTCCTCAGAG TCGGAGAATT ATGAAAGTAC AAAGTCTTAT TATCCAAGGC |
| | • N R Q G A L T V Y T G T V T Q G T D P V • |
| 841 | AAATAGGCAG GGGGCATTAA CTGTTTATAC GGGCACTGTT ACTCAAGGCA CTGACCCCGT |
| | TTTATCCGTC CCCCGTAATT GACAAATATG CCCGTGACAA TGAGTTCCGT GACTGGGGCA |
| | • K T Y Y Q Y T P V S S K A M Y D A Y W N • |
| 901 | TAAAACTTAT TACCAGTACA CTCCTGTATC ATCAAAAGCC ATGTATGACG CTTACTGGAA |
| | ATTTTGAATA ATGGTCATGT GAGGACATAG TAGTTTTCGG TACATACTGC GAATGACCTT |
| | • G K F R D C A F H S G F N E D P F V C E • |
| 961 | CGGTAAATTC AGAGACTGCG CTTTCCATTC TGGCTTTAAT GAAGATCCAT TCGTTTGTGA |
| | GCCATTTAAG TCTCTGACGC GAAAGGTAAG ACCGAAATTA CTTCTAGGTA AGCAAACACT |
| | • Y Q G Q S S D L P Q P P V N A G G G S G • |
| 1021 | ATATCAAGGC CAATCGTCTG ACCTGCCTCA ACCTCCTGTC AATGCTGGCG GCGGCTCTGG |
| | TATAGTTCCG GTTAGCAGAC TGGACGGAGT TGGAGGACAG TTACGACCGC CGCCGAGACC |
| | • G G S G G G S E G G G S E G G G S E G G • |
| 1081 | TGGTGGTTCT GGTGGCGGCT CTGAGGGTGG TGGCTCTGAG GGTGGCGGTT CTGAGGGTGG |
| | ACCACCAAGA CCACCGCCGA GACTCCCACC ACCGAGACTC CCACCGCCAA GACTCCCACC |
| | • G S E G G G S G G G S G S G D F D Y E K • |
| 1141 | CGGCTCTGAG GGAGGCGGTT CCGGTGGTGG CTCTGGTTCC GGTGATTTTG ATTATGAAAA |
| | GCCGAGACTC CCTCCGCCAA GGCCACCACC GAGACCAAGG CCACTAAAAC TAATACTTTT |
| | • M A N A N K G A M T E N A D E N A L Q S • |
| 1201 | GATGGCAAAC GCTAATAAGG GGGCTATGAC CGAAAATGCC GATGAAAACG CGCTACAGTC |
| | CTACCGTTTG CGATTATTCC CCCGATACTG GCTTTTACGG CTACTTTTGC GCGATGTCAG |
| | ClaI |
| | • D A K G K L D S V A T D Y G A A I D G F • |
| 1261 | TGACGCTAAA GGCAAACTTG ATTCTGTCGC TACTGATTAC GGTGCTGCTA TCGATGGTTT |
| | ACTGCGATTT CCGTTTGAAC TAAGACAGCG ATGACTAATG CCACGACGAT AGCTACCAAA |
| | • I G D V S G L A N G N G A T G D F A G S • |
| 1321 | CATTGGTGAC GTTTCCGGCC TTGCTAATGG TAATGGTGCT ACTGGTGATT TTGCTGGCTC |
| | GTAACCACTG CAAAGGCCGG AACGATTACC ATTACCACGA TGACCACTAA AACGACCGAG |
| | • N S Q M A Q V G D G D N S P L M N N F R • |
| 1381 | TAATTCCCAA ATGGCTCAAG TCGGTGACGG TGATAATTCA CCTTTAATGA ATAATTTCCG |
| | ATTAAGGGTT TACCGAGTTC AGCCACTGCC ACTATTAAGT GGAAATTACT TATTAAAGGC |
| | • Q Y L P S L P Q S V E C R P F V F G A G • |
| 1441 | TCAATATTTA CCTTCCCTCC CTCAATCGGT TGAATGTCGC CCTTTTGTCT TTGGCGCTGG |
| | AGTTATAAAT GGAAGGGAGG GAGTTAGCCA ACTTACAGCG GGAAAACAGA AACCGCGACC |
| | • K P Y E F S I D C D K I N L F R G V F A • |
| 1501 | TAAACCATAT GAATTTTCTA TTGATTGTGA CAAAATAAAC TTATTCCGTG GTGTCTTTGC |
| | ATTTGGTATA CTTAAAAGAT AACTAACACT GTTTTATTTG AATAAGGCAC CACAGAAACG |
| | • F L L Y V A T F M Y V F S T F A N I L R • |
| 1561 | GTTTCTTTTA TATGTTGCCA CCTTTATGTA TGTATTTTCT ACGTTTGCTA ACATACTGCG |
| | CAAAGAAAAT ATACAACGGT GGAAATACAT ACATAAAAGA TGCAAACGAT TGTATGACGC |
| | XbaI |
| | • N K E S * |
| 1621 | TAATAAGGAG TCTTAATGAC TCTAGAGGTC GAAATTCACC TCGAAAGCAA GCTGATAAAC |
| | ATTATTCCTC AGAATTACTG AGATCTCCAG CTTTAAGTGG AGCTTTCGTT CGACTATTTG |
| 1681 | CGATACAATT AAAGGCTCCT TTTGGAGCCT TTTTTTTTGG AGATTTTCAA CGTGAAAAAA |
| | GCTATGTTAA TTTCCGAGGA AAACCTCGGA AAAAAAAACC TCTAAAAGTT GCACTTTTTT |
| 1741 | TTATTATTCG CAATTCCAAG CTAATTCACC TCGAAAGCAA GCTGATAAAC CGATACAATT |
| | AATAATAAGC GTTAAGGTTC GATTAAGTGG AGCTTTCGTT CGACTATTTG GCTATGTTAA |
| 1801 | AAAGGCTCCT TTTGGAGCCT TTTTTTTTGG AGATTTTCAA CGTGAAAAAA TTATTATTCG |
| | TTTCCGAGGA AAACCTCGGA AAAAAAAACC TCTAAAAGTT GCACTTTTTT AATAATAAGC |
| 1861 | CAATTCCAAG CTCTGCCTCG CGCGTTTCGG TGATGACGGT GAAAACCTCT GACACATGCA |
| | GTTAAGGTTC GAGACGGAGC GCGCAAAGCC ACTACTGCCA CTTTTGGAGA CTGTGTACGT |
| 1921 | GCTCCCGGAG ACGGTCACAG CTTGTCTGTA AGCGGATGCA GATCACGCGC CCTGTAGCGG |
| | CGAGGGCCTC TGCCAGTGTC GAACAGACAT TCGCCTACGT CTAGTGCGCG GGACATCGCC |
| 1981 | CGCATTAAGC GCGGCGGGTG TGGTGGTTAC GCGCAGCGTG ACCGCTACAC TTGCCAGCGC |
| | GCGTAATTCG CGCCGCCCAC ACCACCAATG CGCGTCGCAC TGGCGATGTG AACGGTCGCG |
| 2041 | CCTAGCGCCC GCTCCTTTCG CTTTCTTCCC TTCCTTTCTC GCCACGTTCG CCAGCTTTCC |
| | GGATCGCGGG CGAGGAAAGC GAAAGAAGGG AAGGAAAGAG CGGTGCAAGC GGTCGAAAGG |
| 2101 | CCGTCAAGCT CTAAATCGGG GGCTCCCTTT AGGGTTCCGA TTTAGTGCTT TACGGCACCT |
| | GGCAGTTCGA GATTTAGCCC CCGAGGGAAA TCCCAAGGCT AAATCACGAA ATGCCGTGGA |
| 2161 | CGACCCCAAA AAACTTGATT AGGGTGATGG TTCACGTAGT GGGCCATCGC CCTGATAGAC |
| | GCTGGGGTTT TTTGAACTAA TCCCACTACC AAGTGCATCA CCCGGTAGCG GGACTATCTG |
| 2221 | GGTTTTTCGC CCTTTGACGT TGGAGTCCAC GTTCTTTAAT AGTGGACTCT TGTTCCAAAC |
| | CCAAAAAGCG GGAAACTGCA ACCTCAGGTG CAAGAAATTA TCACCTGAGA ACAAGGTTTG |
| 2281 | TGGAACAACA CTCAACCCTA TCTCGGTCTA TTCTTTTGAT TTATAAGGGA TTTTGCCGAT |
| | ACCTTGTTGT GAGTTGGGAT AGAGCCAGAT AAGAAAACTA AATATTCCCT AAAACGGCTA |
| 2341 | TTCGGCCTAT TGGTTAAAAA ATGAGCTGAT TTAACAAAAA TTTAACGCGA ATTTTAACAA |
| | AAGCCGGATA ACCAATTTTT TACTCGACTA AATTGTTTTT AAATTGCGCT TAAAATTGTT |
| 2401 | AATATTAACG TTTACAATTT GATCTGCGCT CGGTCGTTCG GCTGCGGCGA GCGGTATCAG |
| | TTATAATTGC AAATGTTAAA CTAGACGCGA GCCAGCAAGC CGACGCCGCT CGCCATAGTC |
| 2461 | CTCACTCAAA GGCGGTAATA CGGTTATCCA CAGAATCAGG GGATAACGCA GGAAAGAACA |
| | GAGTGAGTTT CCGCCATTAT GCCAATAGGT GTCTTAGTCC CCTATTGCGT CCTTTCTTGT |
| 2521 | TGTGAGCAAA AGGCCAGCAA AAGGCCAGGA ACCGTAAAAA GGCCGCGTTG CTGGCGTTTT |
| | ACACTCGTTT TCCGGTCGTT TTCCGGTCCT TGGCATTTTT CCGGCGCAAC GACCGCAAAA |
| 2581 | TCCATAGGCT CCGCCCCCCT GACGAGCATC ACAAAAATCG ACGCTCAAGT CAGAGGTGGC |
| | AGGTATCCGA GGCGGGGGGA CTGCTCGTAG TGTTTTTAGC TGCGAGTTCA GTCTCCACCG |
| 2641 | GAAACCCGAC AGGACTATAA AGATACCAGG CGTTTCCCCC TGGAAGCTCC CTCGTGCGCT |
| | CTTTGGGCTG TCCTGATATT TCTATGGTCC GCAAAGGGGG ACCTTCGAGG GAGCACGCGA |
| 2701 | CTCCTGTTCC GACCCTGCCG CTTACCGGAT ACCTGTCCGC CTTTCTCCCT TCGGGAAGCG |
| | GAGGACAAGG CTGGGACGGC GAATGGCCTA TGGACAGGCG GAAAGAGGGA AGCCCTTCGC |
| 2761 | TGGCGCTTTC TCAATGCTCA CGCTGTAGGT ATCTCAGTTC GGTGTAGGTC GTTCGCTCCA |
| | ACCGCGAAAG AGTTACGAGT GCGACATCCA TAGAGTCAAG CCACATCCAG CAAGCGAGGT |
| | ApaLI |
| 2821 | AGCTGGGCTG TGTGCACGAA CCCCCCGTTC AGCCCGACCG CTGCGCCTTA TCCGGTAACT |
| | TCGACCCGAC ACACGTGCTT GGGGGGCAAG TCGGGCTGGC GACGCGGAAT AGGCCATTGA |
| 2881 | ATCGTCTTGA GTCCAACCCG GTAAGACACG ACTTATCGCC ACTGGCAGCA GCCACTGGTA |
| | TAGCAGAACT CAGGTTGGGC CATTCTGTGC TGAATAGCGG TGACCGTCGT CGGTGACCAT |
| 2941 | ACAGGATTAG CAGAGCGAGG TATGTAGGCG GTGCTACAGA GTTCTTGAAG TGGTGGCCTA |
| | TGTCCTAATC GTCTCGCTCC ATACATCCGC CACGATGTCT CAAGAACTTC ACCACCGGAT |
| 3001 | ACTACGGCTA CACTAGAAGG ACAGTATTTG GTATCTGCGC TCTGCTGAAG CCAGTTACCT |
| | TGATGCCGAT GTGATCTTCC TGTCATAAAC CATAGACGCG AGACGACTTC GGTCAATGGA |
| 3061 | TCGGAAAAAG AGTTGGTAGC TCTTGATCCG GCAAACAAAC CACCGCTGGT AGCGGTGGTT |
| | AGCCTTTTTC TCAACCATCG AGAACTAGGC CGTTTGTTTG GTGGCGACCA TCGCCACCAA |
| 3121 | TTTTTGTTTG CAAGCAGCAG ATTACGCGCA GAAAAAAAGG ATCTCAAGAA GATCCTTTGA |
| | AAAAACAAAC GTTCGTCGTC TAATGCGCGT CTTTTTTTCC TAGAGTTCTT CTAGGAAACT |
| 3181 | TCTTTTCTAC GGGGTCTGAC GCTCAGTGGA ACGAAAACTC ACGTTAAGGG ATTTTGGTCA |
| | AGAAAAGATG CCCCAGACTG CGAGTCACCT TGCTTTTGAG TGCAATTCCC TAAAACCAGT |
| 3241 | TGAGATTATC AAAAAGGATC TTCACCTAGA TCCTTTTAAA TTAAAAATGA AGTTTTAAAT |
| | ACTCTAATAG TTTTTCCTAG AAGTGGATCT AGGAAAATTT AATTTTTACT TCAAAATTTA |
| 3301 | CAATCTAAAG TATATATGAG TAAACTTGGT CTGACAGTTA CCAATGCTTA ATCAGTGAGG |
| | GTTAGATTTC ATATATACTC ATTTGAACCA GACTGTCAAT GGTTACGAAT TAGTCACTCC |
| 3361 | CACCTATCTC AGCGATCTGT CTATTTCGTT CATCCATAGT TGCCTGACTC CCCGTCGTGT |
| | GTGGATAGAG TCGCTAGACA GATAAAGCAA GTAGGTATCA ACGGACTGAG GGGCAGCACA |
| 3421 | AGATAACTAC GATACGGGAG GGCTTACCAT CTGGCCCCAG TGCTGCAATG ATACCGCGAG |
| | TCTATTGATG CTATGCCCTC CCGAATGGTA GACCGGGGTC ACGACGTTAC TATGGCGCTC |
| 3481 | ACCCACGCTC ACCGGCTCCA GATTTATCAG CAATAAACCA GCCAGCCGGA AGGGCCGAGC |
| | TGGGTGCGAG TGGCCGAGGT CTAAATAGTC GTTATTTGGT CGGTCGGCCT TCCCGGCTCG |
| 3541 | GCAGAAGTGG TCCTGCAACT TTATCCGCCT CCATCCAGTC TATTAATTGT TGCCGGGAAG |
| | CGTCTTCACC AGGACGTTGA AATAGGCGGA GGTAGGTCAG ATAATTAACA ACGGCCCTTC |
| | PstI |
| 3601 | CTAGAGTAAG TAGTTCGCCA GTTAATAGTT TGCGCAACGT TGTTGCCATT GCTGCAGGCA |
| | GATCTCATTC ATCAAGCGGT CAATTATCAA ACGCGTTGCA ACAACGGTAA CGACGTCCGT |
| 3661 | TCGTGGTGTC ACGCTCGTCG TTTGGTATGG CTTCATTCAG CTCCGGTTCC CAACGATCAA |
| | AGCACCACAG TGCGAGCAGC AAACCATACC GAAGTAAGTC GAGGCCAAGG GTTGCTAGTT |
| 3721 | GGCGAGTTAC ATGATCCCCC ATGTTGTGCA AAAAAGCGGT TAGCTCCTTC GGTCCTCCGA |
| | CCGCTCAATG TACTAGGGGG TACAACACGT TTTTTCGCCA ATCGAGGAAG CCAGGAGGCT |
| 3781 | TCGTTGTCAG AAGTAAGTTG GCCGCAGTGT TATCACTCAT GGTTATGGCA GCACTGCATA |
| | AGCAACAGTC TTCATTCAAC CGGCGTCACA ATAGTGAGTA CCAATACCGT CGTGACGTAT |
| 3841 | ATTCTCTTAC TGTCATGCCA TCCGTAAGAT GCTTTTCTGT GACTGGTGAG TACTCAACCA |
| | TAAGAGAATG ACAGTACGGT AGGCATTCTA CGAAAAGACA CTGACCACTC ATGAGTTGGT |
| 3901 | AGTCATTCTG AGAATAGTGT ATGCGGCGAC CGAGTTGCTC TTGCCCGGCG TCAACACGGG |
| | TCAGTAAGAC TCTTATCACA TACGCCGCTG GCTCAACGAG AACGGGCCGC AGTTGTGCCC |
| 3961 | ATAATACCGC GCCACATAGC AGAACTTTAA AAGTGCTCAT CATTGGAAAA CGTTCTTCGG |
| | TATTATGGCG CGGTGTATCG TCTTGAAATT TTCACGAGTA GTAACCTTTT GCAAGAAGCC |
| | ApaLI |
| 4021 | GGCGAAAACT CTCAAGGATC TTACCGCTGT TGAGATCCAG TTCGATGTAA CCCACTCGTG |
| | CCGCTTTTGA GAGTTCCTAG AATGGCGACA ACTCTAGGTC AAGCTACATT GGGTGAGCAC |
| | ApaLI |
| 4081 | CACCCAACTG ATCTTCAGCA TCTTTTACTT TCACCAGCGT TTCTGGGTGA GCAAAAACAG |
| | GTGGGTTGAC TAGAAGTCGT AGAAAATGAA AGTGGTCGCA AAGACCCACT CGTTTTTGTC |
| 4141 | GAAGGCAAAA TGCCGCAAAA AAGGGAATAA GGGCGACACG GAAATGTTGA ATACTCATAC |
| | CTTCCGTTTT ACGGCGTTTT TTCCCTTATT CCCGCTGTGC CTTTACAACT TATGAGTATG |
| 4201 | TCTTCCTTTT TCAATATTAT TGAAGCAGAC AGTTTTATTG TTCATGATGA TATATTTTTA |
| | AGAAGGAAAA AGTTATAATA ACTTCGTCTG TCAAAATAAC AAGTACTACT ATATAAAAAT |
| 4261 | TCTTGTGCAA TGTAACATCA GAGATTTTGA GACACAACGT GGCTTTGTTG AATAAATCGA |
| | AGAACACGTT ACATTGTAGT CTCTAAAACT CTGTGTTGCA CCGAAACAAC TTATTTAGCT |
| 4321 | ACTTTTGCTG AGTTGACTCC CCGCGCGCGA TGGGTCGAAT TTGCTTTCGA AAAAAAAGCC |
| | TGAAAACGAC TCAACTGAGG GGCGCGCGCT ACCCAGCTTA AACGAAAGCT TTTTTTTCGG |
| 4381 | CGCTCATTAG GCGGGCTAAA AAAAAGCCCG CTCATTAGGC GGGCTCGAAT TTCTGCCATT |
| | GCGAGTAATC CGCCCGATTT TTTTTCGGGC GAGTAATCCG CCCGAGCTTA AAGACGGTAA |
| 4441 | CATCCGCTTA TTATCACTTA TTCAGGCGTA GCAACCAGGC GTTTAAGGGC ACCAATAACT |
| | GTAGGCGAAT AATAGTGAAT AAGTCCGCAT CGTTGGTCCG CAAATTCCCG TGGTTATTGA |
| 4501 | GCCTTAAAAA AATTACGCCC CGCCCTGCCA CTCATCGCAG TACTGTTGTA ATTCATTAAG |
| | CGGAATTTTT TTAATGCGGG GCGGGACGGT GAGTAGCGTC ATGACAACAT TAAGTAATTC |
| 4561 | CATTCTGCCG ACATGGAAGC CATCACAGAC GGCATGATGA ACCTGAATCG CCAGCGGCAT |
| | GTAAGACGGC TGTACCTTCG GTAGTGTCTG CCGTACTACT TGGACTTAGC GGTCGCCGTA |
| 4621 | CAGCACCTTG TCGCCTTGCG TATAATATTT GCCCATAGTG AAAACGGGGG CGAAGAAGTT |
| | GTCGTGGAAC AGCGGAACGC ATATTATAAA CGGGTATCAC TTTTGCCCCC GCTTCTTCAA |
| 4681 | GTCCATATTC GCCACGTTTA AATCAAAACT GGTGAAACTC ACCCAGGGAT TGGCTGAGAC |
| | CAGGTATAAG CGGTGCAAAT TTAGTTTTGA CCACTTTGAG TGGGTCCCTA ACCGACTCTG |
| 4741 | GAAAAACATA TTCTCAATAA ACCCTTTAGG GAAATAGGCC AGGTTTTCAC CGTAACACGC |
| | CTTTTTGTAT AAGAGTTATT TGGGAAATCC CTTTATCCGG TCCAAAAGTG GCATTGTGCG |
| 4801 | CACATCTTGC GAATATATGT GTAGAAACTG CCGGAAATCG TCGTGGTATT CACTCCAGAG |
| | GTGTAGAACG CTTATATACA CATCTTTGAC GGCCTTTAGC AGCACCATAA GTGAGGTCTC |
| 4861 | CGATGAAAAC GTTTCAGTTT GCTCATGGAA AACGGTGTAA CAAGGGTGAA CACTATCCCA |
| | GCTACTTTTG CAAAGTCAAA CGAGTACCTT TTGCCACATT GTTCCCACTT GTGATAGGGT |
| 4921 | TATCACCAGC TCACCGTCTT TCATTGCCAT ACGAAATTCC GGATGAGCAT TCATCAGGCG |
| | ATAGTGGTCG AGTGGCAGAA AGTAACGGTA TGCTTTAAGG CCTACTCGTA AGTAGTCCGC |
| 4981 | GGCAAGAATG TGAATAAAGG CCGGATAAAA CTTGTGCTTA TTTTTCTTTA CGGTCTTTAA |
| | CCGTTCTTAC ACTTATTTCC GGCCTATTTT GAACACGAAT AAAAAGAAAT GCCAGAAATT |
| 5041 | AAAGGCCGTA ATATCCAGCT GAACGGTCTG GTTATAGGTA CATTGAGCAA CTGACTGAAA |
| | TTTCCGGCAT TATAGGTCGA CTTGCCAGAC CAATATCCAT GTAACTCGTT GACTGACTTT |
| 5101 | TGCCTCAAAA TGTTCTTTAC GATGCCATTG GGATATATCA ACGGTGGTAT ATCCAGTGAT |
| | ACGGAGTTTT ACAAGAAATG CTACGGTAAC CCTATATAGT TGCCACCATA TAGGTCACTA |
| 5161 | TTTTTTCTCC ATTTTAGCTT CCTTAGCTCC TGAAAATCTC GATAACTCAA AAAATACGCC |
| | AAAAAAGAGG TAAAATCGAA GGAATCGAGG ACTTTTAGAG CTATTGAGTT TTTTATGCGG |
| 5221 | CGGTAGTGAT CTTATTTCAT TATGGTGAAA GTTGGAACCT CTTACGTGCC GATCAACGTC |
| | GCCATCACTA GAATAAAGTA ATACCACTTT CAACCTTGGA GAATGCACGG CTAGTTGCAG |
| 5281 | TCATTTTCGC CAAAAGTTGG CCCAGGGCTT CCCGGTATCA ACAGGGACAC CAGGATTTAT |
| | AGTAAAAGCG GTTTTCAACC GGGTCCCGAA GGGCCATAGT TGTCCCTGTG GTCCTAAATA |
| 5341 | TTATTCTGCG AAGTGATCTT CCGTCACAGG TATTTATTCG AAGACGAAAG GGCATCGCGC |
| | AATAAGACGC TTCACTAGAA GGCAGTGTCC ATAAATAAGC TTCTGCTTTC CCGTAGCGCG |
| 5401 | GCGGGGAATT GGCCACGATG CGTCCGGCGT AGAGGATCTC TCACCTACCA AACAATGCCC |
| | CGCCCCTTAA CCGGTGCTAC GCAGGCCGCA TCTCCTAGAG AGTGGATGGT TTGTTACGGG |
| 5461 | CCCTGCAAAA AATAAATTCA TATAAAAAAC ATACAGATAA CCATCTGCGG TGATAAATTA |
| | GGGACGTTTT TTATTTAAGT ATATTTTTTG TATGTCTATT GGTAGACGCC ACTATTTAAT |
| 5521 | TCTCTGGCGG TGTTGACATA AATACCACTG GCGGTGATAC TGAGCACATC AGCAGGACGC |
| | AGAGACCGCC ACAACTGTAT TTATGGTGAC CGCCACTATG ACTCGTGTAG TCGTCCTGCG |
| 5581 | ACTGACCACC ATGAAGGTG |
| | TGACTGGTGG TACTTCCAC |
| Step 4b Out pEVO_7.vec | |
| 1 | ACGCTCTTAA AATTAAGCCC TGAAGAAGGG CAGCATTCAA AGCAGAAGGC TTTGGGGTGT |
| | TGCGAGAATT TTAATTCGGG ACTTCTTCCC GTCGTAAGTT TCGTCTTCCG AAACCCCACA |
| | EcoRI |
| 61 | GTGATACGAA ACGAAGCATT GGAATTCTAC AACTTGCTTG GATTCCTACA AAGAAGCAGC |
| | CACTATGCTT TGCTTCGTAA CCTTAAGATG TTGAACGAAC CTAAGGATGT TTCTTCGTCG |
| | XbaI |
| | M K K • |
| 121 | AATTTTCAGT GTCAGAAGTC GACCAAGGAG GTCTAGATAA CGAGGGCAAA AAATGAAAAA |
| | TTAAAAGTCA CAGTCTTCAG CTGGTTCCTC CAGATCTATT GCTCCCGTTT TTTACTTTTT |
| | SacI |
| | • T A I A I A V A L A G F A T V A Q A E L • |
| 181 | GACAGCTATC GCGATTGCAG TGGCACTGGC TGGTTTCGCT ACCGTAGCGC AGGCCGAGCT |
| | CTGTCGATAG CGCTAACGTC ACCGTGACCG ACCAAAGCGA TGGCATCGCG TCCGGCTCGA |
| | SacI BglII KpnI AvaI |
| | • A G C T K I Y D P V C G T A G G G G S G • |
| 241 | CGCCGGTTGC ACTAAGATCT ACGACCCGGT TTGCGGTACC GCCGGCGGCG GTGGCTCGGG |
| | GCGGCCAACG TGATTCTAGA TGCTGGGCCA AACGCCATGG CGGCCGCCGC CACCGAGCCC |
| | • G G G G G G A P T Y P P P P P P G G S G • |
| 301 | CGGTGGCGGA GGGGGCGGGG CGCCGACTTA TCCTCCCCCT CCCCCTCCCG GAGGCTCTGG |
| | GCCACCGCCT CCCCCGCCCC GCGGCTGAAT AGGAGGGGGA GGGGGAGGGC CTCCGAGACC |
| | EcoRI EcoRV |
| | • G G L I H E E G E F S E A R E D I R A E • |
| 361 | GGGGGGCTTA ATTCATGAAG AAGGTGAATT CTCAGAAGCG CGCGAAGATA TCAGAGCTGA |
| | CCCCCCGAAT TAAGTACTTC TTCCACTTAA GAGTCTTCGC GCGCTTCTAT AGTCTCGACT |
| | • T V E S C L A K S H T E N S F T N V W K • |
| 421 | AACTGTTGAA AGTTGTTTAG CAAAATCCCA TACAGAAAAT TCATTTACTA ACGTCTGGAA |
| | TTGACAACTT TCAACAAATC GTTTTAGGGT ATGTCTTTTA AGTAAATGAT TGCAGACCTT |
| | • D D K T L D R Y A N Y E G C L W N A T G • |
| 481 | AGACGACAAA ACTTTAGATC GTTACGCTAA CTATGAGGGC TGTCTGTGGA ATGCTACAGG |
| | TCTGCTGTTT TGAAATCTAG CAATGCGATT GATACTCCCG ACAGACACCT TACGATGTCC |
| | • V V V C T G D E T Q C Y G T W V P I G L • |
| 541 | CGTTGTAGTT TGTACTGGTG ACGAAACTCA GTGTTACGGT ACATGGGTTC CTATTGGGCT |
| | GCAACATCAA ACATGACCAC TGCTTTGAGT CACAATGCCA TGTACCCAAG GATAACCCGA |
| | • A I P E N E G G G S E G G G S E G G G S • |
| 601 | TGCTATCCCT GAAAATGAGG GTGGTGGCTC TGAGGGTGGC GGTTCTGAGG GTGGCGGTTC |
| | ACGATAGGGA CTTTTACTCC CACCACCGAG ACTCCCACCG CCAAGACTCC CACCGCCAAG |
| | • E G G G T K P P E Y G D T P I P G Y T Y • |
| 661 | TGAGGGTGGC GGTACTAAAC CTCCTGAGTA CGGTGATACA CCTATTCCGG GCTATACTTA |
| | ACTCCCACCG CCATGATTTG GAGGACTCAT GCCACTATGT GGATAAGGCC CGATATGAAT |
| | • I N P L D G T Y P P G T E Q N P A N P N • |
| 721 | TATCAACCCT CTCGACGGCA CTTATCCGCC TGGTACTGAG CAAAACCCCG CTAATCCTAA |
| | ATAGTTGGGA GAGCTGCCGT GAATAGGCGG ACCATGACTC GTTTTGGGGC GATTAGGATT |
| | • P S L E E S Q P L N T F M F Q N N R F R • |
| 781 | TCCTTCTCTT GAGGAGTCTC AGCCTCTTAA TACTTTCATG TTTCAGAATA ATAGGTTCCG |
| | AGGAAGAGAA CTCCTCAGAG TCGGAGAATT ATGAAAGTAC AAAGTCTTAT TATCCAAGGC |
| | • N R Q G A L T V Y T G T V T Q G T D P V • |
| 841 | AAATAGGCAG GGGGCATTAA CTGTTTATAC GGGCACTGTT ACTCAAGGCA CTGACCCCGT |
| | TTTATCCGTC CCCCGTAATT GACAAATATG CCCGTGACAA TGAGTTCCGT GACTGGGGCA |
| | • K T Y Y Q Y T P V S S K A M Y D A Y W N • |
| 901 | TAAAACTTAT TACCAGTACA CTCCTGTATC ATCAAAAGCC ATGTATGACG CTTACTGGAA |
| | ATTTTGAATA ATGGTCATGT GAGGACATAG TAGTTTTCGG TACATACTGC GAATGACCTT |
| | • G K F R D C A F H S G F N E D P F V C E • |
| 961 | CGGTAAATTC AGAGACTGCG CTTTCCATTC TGGCTTTAAT GAAGATCCAT TCGTTTGTGA |
| | GCCATTTAAG TCTCTGACGC GAAAGGTAAG ACCGAAATTA CTTCTAGGTA AGCAAACACT |
| | • Y Q G Q S S D L P Q P P V N A G G G S G • |
| 1021 | ATATCAAGGC CAATCGTCTG ACCTGCCTCA ACCTCCTGTC AATGCTGGCG GCGGCTCTGG |
| | TATAGTTCCG GTTAGCAGAC TGGACGGAGT TGGAGGACAG TTACGACCGC CGCCGAGACC |
| | • G G S G G G S E G G G S E G G G S E G G • |
| 1081 | TGGTGGTTCT GGTGGCGGCT CTGAGGGTGG TGGCTCTGAG GGTGGCGGTT CTGAGGGTGG |
| | ACCACCAAGA CCACCGCCGA GACTCCCACC ACCGAGACTC CCACCGCCAA GACTCCCACC |
| | • G S E G G G S G G G S G S G D F D Y E K • |
| 1141 | CGGCTCTGAG GGAGGCGGTT CCGGTGGTGG CTCTGGTTCC GGTGATTTTG ATTATGAAAA |
| | GCCGAGACTC CCTCCGCCAA GGCCACCACC GAGACCAAGG CCACTAAAAC TAATACTTTT |
| | • M A N A N K G A M T E N A D E N A L Q S • |
| 1201 | GATGGCAAAC GCTAATAAGG GGGCTATGAC CGAAAATGCC GATGAAAACG CGCTACAGTC |
| | CTACCGTTTG CGATTATTCC CCCGATACTG GCTTTTACGG CTACTTTTGC GCGATGTCAG |
| | ClaI |
| | • D A K G K L D S V A T D Y G A A I D G F • |
| 1261 | TGACGCTAAA GGCAAACTTG ATTCTGTCGC TACTGATTAC GGTGCTGCTA TCGATGGTTT |
| | ACTGCGATTT CCGTTTGAAC TAAGACAGCG ATGACTAATG CCACGACGAT AGCTACCAAA |
| | • I G D V S G L A N G N G A T G D F A G S • |
| 1321 | CATTGGTGAC GTTTCCGGCC TTGCTAATGG TAATGGTGCT ACTGGTGATT TTGCTGGCTC |
| | GTAACCACTG CAAAGGCCGG AACGATTACC ATTACCACGA TGACCACTAA AACGACCGAG |
| | • N S Q M A Q V G D G D N S P L M N N F R • |
| 1381 | TAATTCCCAA ATGGCTCAAG TCGGTGACGG TGATAATTCA CCTTTAATGA ATAATTTCCG |
| | ATTAAGGGTT TACCGAGTTC AGCCACTGCC ACTATTAAGT GGAAATTACT TATTAAAGGC |
| | • Q Y L P S L P Q S V E C R P F V F G A G • |
| 1441 | TCAATATTTA CCTTCCCTCC CTCAATCGGT TGAATGTCGC CCTTTTGTCT TTGGCGCTGG |
| | AGTTATAAAT GGAAGGGAGG GAGTTAGCCA ACTTACAGCG GGAAAACAGA AACCGCGACC |
| | • K P Y E F S I D C D K I N L F R G V F A • |
| 1501 | TAAACCATAT GAATTTTCTA TTGATTGTGA CAAAATAAAC TTATTCCGTG GTGTCTTTGC |
| | ATTTGGTATA CTTAAAAGAT AACTAACACT GTTTTATTTG AATAAGGCAC CACAGAAACG |
| | • F L L Y V A T F M Y V F S T F A N I L R • |
| 1561 | GTTTCTTTTA TATGTTGCCA CCTTTATGTA TGTATTTTCT ACGTTTGCTA ACATACTGCG |
| | CAAAGAAAAT ATACAACGGT GGAAATACAT ACATAAAAGA TGCAAACGAT TGTATGACGC |
| | XbaI |
| | • N K E S * |
| 1621 | TAATAAGGAG TCTTAATGAC TCTAGAGGTC GAAATTCACC TCGAAAGCAA GCTGATAAAC |
| | ATTATTCCTC AGAATTACTG AGATCTCCAG CTTTAAGTGG AGCTTTCGTT CGACTATTTG |
| 1681 | CGATACAATT AAAGGCTCCT TTTGGAGCCT TTTTTTTTGG AGATTTTCAA CGTGAAAAAA |
| | GCTATGTTAA TTTCCGAGGA AAACCTCGGA AAAAAAAACC TCTAAAAGTT GCACTTTTTT |
| 1741 | TTATTATTCG CAATTCCAAG CTAATTCACC TCGAAAGCAA GCTGATAAAC CGATACAATT |
| | AATAATAAGC GTTAAGGTTC GATTAAGTGG AGCTTTCGTT CGACTATTTG GCTATGTTAA |
| 1801 | AAAGGCTCCT TTTGGAGCCT TTTTTTTTGG AGATTTTCAA CGTGAAAAAA TTATTATTCG |
| | TTTCCGAGGA AAACCTCGGA AAAAAAAACC TCTAAAAGTT GCACTTTTTT AATAATAAGC |
| 1861 | CAATTCCAAG CTCTGCCTCG CGCGTTTCGG TGATGACGGT GAAAACCTCT GACACATGCA |
| | GTTAAGGTTC GAGACGGAGC GCGCAAAGCC ACTACTGCCA CTTTTGGAGA CTGTGTACGT |
| 1921 | GCTCCCGGAG ACGGTCACAG CTTGTCTGTA AGCGGATGCA GATCACGCGC CCTGTAGCGG |
| | CGAGGGCCTC TGCCAGTGTC GAACAGACAT TCGCCTACGT CTAGTGCGCG GGACATCGCC |
| 1981 | CGCATTAAGC GCGGCGGGTG TGGTGGTTAC GCGCAGCGTG ACCGCTACAC TTGCCAGCGC |
| | GCGTAATTCG CGCCGCCCAC ACCACCAATG CGCGTCGCAC TGGCGATGTG AACGGTCGCG |
| 2041 | CCTAGCGCCC GCTCCTTTCG CTTTCTTCCC TTCCTTTCTC GCCACGTTCG CCAGCTTTCC |
| | GGATCGCGGG CGAGGAAAGC GAAAGAAGGG AAGGAAAGAG CGGTGCAAGC GGTCGAAAGG |
| 2101 | CCGTCAAGCT CTAAATCGGG GGCTCCCTTT AGGGTTCCGA TTTAGTGCTT TACGGCACCT |
| | GGCAGTTCGA GATTTAGCCC CCGAGGGAAA TCCCAAGGCT AAATCACGAA ATGCCGTGGA |
| 2161 | CGACCCCAAA AAACTTGATT AGGGTGATGG TTCACGTAGT GGGCCATCGC CCTGATAGAC |
| | GCTGGGGTTT TTTGAACTAA TCCCACTACC AAGTGCATCA CCCGGTAGCG GGACTATCTG |
| 2221 | GGTTTTTCGC CCTTTGACGT TGGAGTCCAC GTTCTTTAAT AGTGGACTCT TGTTCCAAAC |
| | CCAAAAAGCG GGAAACTGCA ACCTCAGGTG CAAGAAATTA TCACCTGAGA ACAAGGTTTG |
| 2281 | TGGAACAACA CTCAACCCTA TCTCGGTCTA TTCTTTTGAT TTATAAGGGA TTTTGCCGAT |
| | ACCTTGTTGT GAGTTGGGAT AGAGCCAGAT AAGAAAACTA AATATTCCCT AAAACGGCTA |
| 2341 | TTCGGCCTAT TGGTTAAAAA ATGAGCTGAT TTAACAAAAA TTTAACGCGA ATTTTAACAA |
| | AAGCCGGATA ACCAATTTTT TACTCGACTA AATTGTTTTT AAATTGCGCT TAAAATTGTT |
| 2401 | AATATTAACG TTTACAATTT GATCTGCGCT CGGTCGTTCG GCTGCGGCGA GCGGTATCAG |
| | TTATAATTGC AAATGTTAAA CTAGACGCGA GCCAGCAAGC CGACGCCGCT CGCCATAGTC |
| 2461 | CTCACTCAAA GGCGGTAATA CGGTTATCCA CAGAATCAGG GGATAACGCA GGAAAGAACA |
| | GAGTGAGTTT CCGCCATTAT GCCAATAGGT GTCTTAGTCC CCTATTGCGT CCTTTCTTGT |
| 2521 | TGTGAGCAAA AGGCCAGCAA AAGGCCAGGA ACCGTAAAAA GGCCGCGTTG CTGGCGTTTT |
| | ACACTCGTTT TCCGGTCGTT TTCCGGTCCT TGGCATTTTT CCGGCGCAAC GACCGCAAAA |
| 2581 | TCCATAGGCT CCGCCCCCCT GACGAGCATC ACAAAAATCG ACGCTCAAGT CAGAGGTGGC |
| | AGGTATCCGA GGCGGGGGGA CTGCTCGTAG TGTTTTTAGC TGCGAGTTCA GTCTCCACCG |
| 2641 | GAAACCCGAC AGGACTATAA AGATACCAGG CGTTTCCCCC TGGAAGCTCC CTCGTGCGCT |
| | CTTTGGGCTG TCCTGATATT TCTATGGTCC GCAAAGGGGG ACCTTCGAGG GAGCACGCGA |
| 2701 | CTCCTGTTCC GACCCTGCCG CTTACCGGAT ACCTGTCCGC CTTTCTCCCT TCGGGAAGCG |
| | GAGGACAAGG CTGGGACGGC GAATGGCCTA TGGACAGGCG GAAAGAGGGA AGCCCTTCGC |
| 2761 | TGGCGCTTTC TCAATGCTCA CGCTGTAGGT ATCTCAGTTC GGTGTAGGTC GTTCGCTCCA |
| | ACCGCGAAAG AGTTACGAGT GCGACATCCA TAGAGTCAAG CCACATCCAG CAAGCGAGGT |
| | ApaLI |
| 2821 | AGCTGGGCTG TGTGCACGAA CCCCCCGTTC AGCCCGACCG CTGCGCCTTA TCCGGTAACT |
| | TCGACCCGAC ACACGTGCTT GGGGGGCAAG TCGGGCTGGC GACGCGGAAT AGGCCATTGA |
| 2881 | ATCGTCTTGA GTCCAACCCG GTAAGACACG ACTTATCGCC ACTGGCAGCA GCCACTGGTA |
| | TAGCAGAACT CAGGTTGGGC CATTCTGTGC TGAATAGCGG TGACCGTCGT CGGTGACCAT |
| 2941 | ACAGGATTAG CAGAGCGAGG TATGTAGGCG GTGCTACAGA GTTCTTGAAG TGGTGGCCTA |
| | TGTCCTAATC GTCTCGCTCC ATACATCCGC CACGATGTCT CAAGAACTTC ACCACCGGAT |
| 3001 | ACTACGGCTA CACTAGAAGG ACAGTATTTG GTATCTGCGC TCTGCTGAAG CCAGTTACCT |
| | TGATGCCGAT GTGATCTTCC TGTCATAAAC CATAGACGCG AGACGACTTC GGTCAATGGA |
| 3061 | TCGGAAAAAG AGTTGGTAGC TCTTGATCCG GCAAACAAAC CACCGCTGGT AGCGGTGGTT |
| | AGCCTTTTTC TCAACCATCG AGAACTAGGC CGTTTGTTTG GTGGCGACCA TCGCCACCAA |
| 3121 | TTTTTGTTTG CAAGCAGCAG ATTACGCGCA GAAAAAAAGG ATCTCAAGAA GATCCTTTGA |
| | AAAAACAAAC GTTCGTCGTC TAATGCGCGT CTTTTTTTCC TAGAGTTCTT CTAGGAAACT |
| 3181 | TCTTTTCTAC GGGGTCTGAC GCTCAGTGGA ACGAAAACTC ACGTTAAGGG ATTTTGGTCA |
| | AGAAAAGATG CCCCAGACTG CGAGTCACCT TGCTTTTGAG TGCAATTCCC TAAAACCAGT |
| 3241 | TGAGATTATC AAAAAGGATC TTCACCTAGA TCCTTTTAAA TTAAAAATGA AGTTTTAAAT |
| | ACTCTAATAG TTTTTCCTAG AAGTGGATCT AGGAAAATTT AATTTTTACT TCAAAATTTA |
| 3301 | CAATCTAAAG TATATATGAG TAAACTTGGT CTGACAGTTA CCAATGCTTA ATCAGTGAGG |
| | GTTAGATTTC ATATATACTC ATTTGAACCA GACTGTCAAT GGTTACGAAT TAGTCACTCC |
| 3361 | CACCTATCTC AGCGATCTGT CTATTTCGTT CATCCATAGT TGCCTGACTC CCCGTCGTGT |
| | GTGGATAGAG TCGCTAGACA GATAAAGCAA GTAGGTATCA ACGGACTGAG GGGCAGCACA |
| 3421 | AGATAACTAC GATACGGGAG GGCTTACCAT CTGGCCCCAG TGCTGCAATG ATACCGCGAG |
| | TCTATTGATG CTATGCCCTC CCGAATGGTA GACCGGGGTC ACGACGTTAC TATGGCGCTC |
| 3481 | ACCCACGCTC ACCGGCTCCA GATTTATCAG CAATAAACCA GCCAGCCGGA AGGGCCGAGC |
| | TGGGTGCGAG TGGCCGAGGT CTAAATAGTC GTTATTTGGT CGGTCGGCCT TCCCGGCTCG |
| 3541 | GCAGAAGTGG TCCTGCAACT TTATCCGCCT CCATCCAGTC TATTAATTGT TGCCGGGAAG |
| | CGTCTTCACC AGGACGTTGA AATAGGCGGA GGTAGGTCAG ATAATTAACA ACGGCCCTTC |
| | PstI |
| 3601 | CTAGAGTAAG TAGTTCGCCA GTTAATAGTT TGCGCAACGT TGTTGCCATT GCTGCAGGCA |
| | GATCTCATTC ATCAAGCGGT CAATTATCAA ACGCGTTGCA ACAACGGTAA CGACGTCCGT |
| 3661 | TCGTGGTGTC ACGCTCGTCG TTTGGTATGG CTTCATTCAG CTCCGGTTCC CAACGATCAA |
| | AGCACCACAG TGCGAGCAGC AAACCATACC GAAGTAAGTC GAGGCCAAGG GTTGCTAGTT |
| 3721 | GGCGAGTTAC ATGATCCCCC ATGTTGTGCA AAAAAGCGGT TAGCTCCTTC GGTCCTCCGA |
| | CCGCTCAATG TACTAGGGGG TACAACACGT TTTTTCGCCA ATCGAGGAAG CCAGGAGGCT |
| 3781 | TCGTTGTCAG AAGTAAGTTG GCCGCAGTGT TATCACTCAT GGTTATGGCA GCACTGCATA |
| | AGCAACAGTC TTCATTCAAC CGGCGTCACA ATAGTGAGTA CCAATACCGT CGTGACGTAT |
| 3841 | ATTCTCTTAC TGTCATGCCA TCCGTAAGAT GCTTTTCTGT GACTGGTGAG TACTCAACCA |
| | TAAGAGAATG ACAGTACGGT AGGCATTCTA CGAAAAGACA CTGACCACTC ATGAGTTGGT |
| 3901 | AGTCATTCTG AGAATAGTGT ATGCGGCGAC CGAGTTGCTC TTGCCCGGCG TCAACACGGG |
| | TCAGTAAGAC TCTTATCACA TACGCCGCTG GCTCAACGAG AACGGGCCGC AGTTGTGCCC |
| 3961 | ATAATACCGC GCCACATAGC AGAACTTTAA AAGTGCTCAT CATTGGAAAA CGTTCTTCGG |
| | TATTATGGCG CGGTGTATCG TCTTGAAATT TTCACGAGTA GTAACCTTTT GCAAGAAGCC |
| | ApaLI |
| | ˜˜˜ |
| 4021 | GGCGAAAACT CTCAAGGATC TTACCGCTGT TGAGATCCAG TTCGATGTAA CCCACTCGTG |
| | CCGCTTTTGA GAGTTCCTAG AATGGCGACA ACTCTAGGTC AAGCTACATT GGGTGAGCAC |
| | ApaLI |
| 4081 | CACCCAACTG ATCTTCAGCA TCTTTTACTT TCACCAGCGT TTCTGGGTGA GCAAAAACAG |
| | GTGGGTTGAC TAGAAGTCGT AGAAAATGAA AGTGGTCGCA AAGACCCACT CGTTTTTGTC |
| 4141 | GAAGGCAAAA TGCCGCAAAA AAGGGAATAA GGGCGACACG GAAATGTTGA ATACTCATAC |
| | CTTCCGTTTT ACGGCGTTTT TTCCCTTATT CCCGCTGTGC CTTTACAACT TATGAGTATG |
| 4201 | TCTTCCTTTT TCAATATTAT TGAAGCAGAC AGTTTTATTG TTCATGATGA TATATTTTTA |
| | AGAAGGAAAA AGTTATAATA ACTTCGTCTG TCAAAATAAC AAGTACTACT ATATAAAAAT |
| 4261 | TCTTGTGCAA TGTAACATCA GAGATTTTGA GACACAACGT GGCTTTGTTG AATAAATCGA |
| | AGAACACGTT ACATTGTAGT CTCTAAAACT CTGTGTTGCA CCGAAACAAC TTATTTAGCT |
| 4321 | ACTTTTGCTG AGTTGACTCC CCGCGCGCGA TGGGTCGAAT TTGCTTTCGA AAAAAAAGCC |
| | TGAAAACGAC TCAACTGAGG GGCGCGCGCT ACCCAGCTTA AACGAAAGCT TTTTTTTCGG |
| 4381 | CGCTCATTAG GCGGGCTAAA AAAAAGCCCG CTCATTAGGC GGGCTCGAAT TTCTGCCATT |
| | GCGAGTAATC CGCCCGATTT TTTTTCGGGC GAGTAATCCG CCCGAGCTTA AAGACGGTAA |
| 4441 | CATCCGCTTA TTATCACTTA TTCAGGCGTA GCAACCAGGC GTTTAAGGGC ACCAATAACT |
| | GTAGGCGAAT AATAGTGAAT AAGTCCGCAT CGTTGGTCCG CAAATTCCCG TGGTTATTGA |
| 4501 | GCCTTAAAAA AATTACGCCC CGCCCTGCCA CTCATCGCAG TACTGTTGTA ATTCATTAAG |
| | CGGAATTTTT TTAATGCGGG GCGGGACGGT GAGTAGCGTC ATGACAACAT TAAGTAATTC |
| 4561 | CATTCTGCCG ACATGGAAGC CATCACAGAC GGCATGATGA ACCTGAATCG CCAGCGGCAT |
| | GTAAGACGGC TGTACCTTCG GTAGTGTCTG CCGTACTACT TGGACTTAGC GGTCGCCGTA |
| 4621 | CAGCACCTTG TCGCCTTGCG TATAATATTT GCCCATAGTG AAAACGGGGG CGAAGAAGTT |
| | GTCGTGGAAC AGCGGAACGC ATATTATAAA CGGGTATCAC TTTTGCCCCC GCTTCTTCAA |
| 4681 | GTCCATATTC GCCACGTTTA AATCAAAACT GGTGAAACTC ACCCAGGGAT TGGCTGAGAC |
| | CAGGTATAAG CGGTGCAAAT TTAGTTTTGA CCACTTTGAG TGGGTCCCTA ACCGACTCTG |
| 4741 | GAAAAACATA TTCTCAATAA ACCCTTTAGG GAAATAGGCC AGGTTTTCAC CGTAACACGC |
| | CTTTTTGTAT AAGAGTTATT TGGGAAATCC CTTTATCCGG TCCAAAAGTG GCATTGTGCG |
| 4801 | CACATCTTGC GAATATATGT GTAGAAACTG CCGGAAATCG TCGTGGTATT CACTCCAGAG |
| | GTGTAGAACG CTTATATACA CATCTTTGAC GGCCTTTAGC AGCACCATAA GTGAGGTCTC |
| 4861 | CGATGAAAAC GTTTCAGTTT GCTCATGGAA AACGGTGTAA CAAGGGTGAA CACTATCCCA |
| | GCTACTTTTG CAAAGTCAAA CGAGTACCTT TTGCCACATT GTTCCCACTT GTGATAGGGT |
| 4921 | TATCACCAGC TCACCGTCTT TCATTGCCAT ACGAAATTCC GGATGAGCAT TCATCAGGCG |
| | ATAGTGGTCG AGTGGCAGAA AGTAACGGTA TGCTTTAAGG CCTACTCGTA AGTAGTCCGC |
| 4981 | GGCAAGAATG TGAATAAAGG CCGGATAAAA CTTGTGCTTA TTTTTCTTTA CGGTCTTTAA |
| | CCGTTCTTAC ACTTATTTCC GGCCTATTTT GAACACGAAT AAAAAGAAAT GCCAGAAATT |
| 5041 | AAAGGCCGTA ATATCCAGCT GAACGGTCTG GTTATAGGTA CATTGAGCAA CTGACTGAAA |
| | TTTCCGGCAT TATAGGTCGA CTTGCCAGAC CAATATCCAT GTAACTCGTT GACTGACTTT |
| 5101 | TGCCTCAAAA TGTTCTTTAC GATGCCATTG GGATATATCA ACGGTGGTAT ATCCAGTGAT |
| | ACGGAGTTTT ACAAGAAATG CTACGGTAAC CCTATATAGT TGCCACCATA TAGGTCACTA |
| 5161 | TTTTTTCTCC ATTTTAGCTT CCTTAGCTCC TGAAAATCTC GATAACTCAA AAAATACGCC |
| | AAAAAAGAGG TAAAATCGAA GGAATCGAGG ACTTTTAGAG CTATTGAGTT TTTTATGCGG |
| 5221 | CGGTAGTGAT CTTATTTCAT TATGGTGAAA GTTGGAACCT CTTACGTGCC GATCAACGTC |
| | GCCATCACTA GAATAAAGTA ATACCACTTT CAACCTTGGA GAATGCACGG CTAGTTGCAG |
| 5281 | TCATTTTCGC CAAAAGTTGG CCCAGGGCTT CCCGGTATCA ACAGGGACAC CAGGATTTAT |
| | AGTAAAAGCG GTTTTCAACC GGGTCCCGAA GGGCCATAGT TGTCCCTGTG GTCCTAAATA |
| 5341 | TTATTCTGCG AAGTGATCTT CCGTCACAGG TATTTATTCG AAGACGAAAG GGCATCGCGC |
| | AATAAGACGC TTCACTAGAA GGCAGTGTCC ATAAATAAGC TTCTGCTTTC CCGTAGCGCG |
| 5401 | GCGGGGAATT GGCCACGATG CGTCCGGCGT AGAGGATCTC TCACCTACCA AACAATGCCC |
| | CGCCCCTTAA CCGGTGCTAC GCAGGCCGCA TCTCCTAGAG AGTGGATGGT TTGTTACGGG |
| 5461 | CCCTGCAAAA AATAAATTCA TATAAAAAAC ATACAGATAA CCATCTGCGG TGATAAATTA |
| | GGGACGTTTT TTATTTAAGT ATATTTTTTG TATGTCTATT GGTAGACGCC ACTATTTAAT |
| 5521 | TCTCTGGCGG TGTTGACATA AATACCACTG GCGGTGATAC TGAGCACATC AGCAGGACGC |
| | AGAGACCGCC ACAACTGTAT TTATGGTGAC CGCCACTATG ACTCGTGTAG TCGTCCTGCG |
| 5581 | ACTGACCACC ATGAAGGTG |
| | TGACTGGTGG TACTTCCAC |

WT SCCE Preparation

### “SCCE_Gly_His6” into pIE1/153A (V4) {insect cell vector}

Template:

Invitrogen Clone ID: 45750452

Organism: Homo sapiens

Matching Nucleotide Accession: NM_{—}005046

Primers Used:

SCCE BamH 1 F:
CCCGGATCCATGGCAAGATCCCTTCTCCTGCCCC

The highlight (Arial type face) shows the leading portion of the forward primer that lays down on the template.

SCCE BstXI His Gly R:
GGAGCTCCACCGCGGTGGCGTTAATGATGATGATGATGATGACCGCCGCC
CCCGCCGCCGCGGCCGCCGCGATGCTTTTTCATGGTGTCATTTATCC

The highlight (Tahoma type face) shows the leading portion of the reverse primer that lays down on the template.
Method: Sub-cloning using unique Restriction Sites
Preparation of Vector:
pIE 10 μg (X μl)
10×NEB R.E. Buffer for BamHI 6 μl
BSA 0.6 μl
R.E. BamHI 3 μl
R.E. BstXI 3 μl
H₂O up to 60 μl
37° C. for 3 hours
Add 1 μl (1 U/μl) Alakaline phosphotase (Roche)
37° C. for 1 hour
Phenol Chloroform Extract
Purify digested vector by running on an agarose get and use QIAquick Gel Extraction Kit
Run aliquot of eluate (purified digested vector) for quantitation
Preparation of Insert:
1 μl of SCCE BamH 1 F (1 μg/μl)
1 μl of SCCE BstX1 His Gly R (1 μg/μl)
250 ng (X μl) template (Clone ID 45750452 from Invitrogen)
10 μl 10×TAQ Polymerase Buffer from NEB
0.8 μl dNTP (25 mM)
H₂O to a final volume of 100 μl
95° C. for 5 min
Ice; microfuge
Then add 1 μl of TAQ DNA Polymerase (NEB)
Step 1: 95° C. 30 seconds
Step 2: 95° C. 30 seconds

58° C. 1 minute

72° C. 1 minute/kb of pcr product length (1 min for SCCE)

Repest step#2 29 times

Step 3: 72° C. for 10 min

Step 4: 4° C. pause

Check pcr by electrophoresis of 5 μl of the pcr product on a 1% agarose gel.

Purify the pcr product by using a QIAquick pcr purification Kit

Elute in 50 μl of elution buffer

Pcr product 50 μl

10×NEB R.E. Buffer for BamHI 7 μl

BSA 0.7 μl

R.E. BamHI 3 μl

R.E. BstXI 3 μl

H₂O up to 70 μl

37° C. overnight

Phenol Chloroform Extract

Purify digested pcr by running on an agarose get and use QIAquick Gel Extraction Kit

Run aliquot of eluate (purified digested insert) for quantitation

Ligation of Vector and Insert

Vector: pIE/153A (V4) digested with BamHI & BstXI

Insert: pcr product digested with BamHI & BstXI

| | | 10× | | |
|---|---|---|---|---|
| Vector | Insert | Ligation | | |
| (fmol) | (fmol) | Buffer (μl) | H₂O (μl) | Ligase (μl) |
| 30 | 60 | 2.5 | Upto 25 | 0.5 |
| 30 | 150 | 2.5 | Upto 25 | 0.5 |
| 30 | 0 | 2.5 | Upto 25 | 0.5 |

12° C. for 16 hours (overnight)
Transformation of Ligation Product into Competent C 7118 Cells
1. Gently thaw the competent C 7118 cells on ice. For each control and sample reaction to be transformed, aliquot 50 μl of the competent cells to a prechilled 15 ml conical tube.
2. Transfer 25 μl of each ligation product to separate aliquots of the competent cells. Swirl the transformation reactions gently to mix and incubate the reactions on ice for 30 minutes.
3. Heat pulse the transformation reactions for 3 min 37° C. and then place the reactions on ice for 2 minutes.
4. Add 0.8 ml of SOC medium and incubate the transformation reactions at 37° C. for 1.5 hours with shaking at 200-250 rpm.
5. Spin the tube at 3000 rpm for 5 min and re-suspend cells in 200 μl LB medium. Plate the cells on agar plates containing the ampicillin.
6. Incubate the transformation plates at 37° C. for >16 hours.
7. Next day, pick up a single colony, grow overnight in 3 ml LB medium.
8. Use QIAprep spin miniprep kit for plasmid purification.

9. The sequence was confirmed with the following sequencing primers:

| pIE Seq F: GACGAAGAAGTTGCCGCGTTGG | |
|---|---|
| pIE Seq R: CGATGGTGATGACCTGACCGTC | |
| Sequence pIE WT SCCE | |
| 1 | CAGCTTTTGT TCCCTTTAGT GAGGGTTAAT TCCGAGCTTG GCGTAATCAT GGTCATAGCT |
| | GTCGAAAACA AGGGAAATCA CTCCCAATTA AGGCTCGAAC CGCATTAGTA CCAGTATCGA |
| 61 | GTTTCCTGTG TGAAATTGTT ATCCGCTCAC AATTCCACAC AACATACGAG CCGGAAGCAT |
| | CAAAGGACAC ACTTTAACAA TAGGCGAGTG TTAAGGTGTG TTGTATGCTC GGCCTTCGTA |
| 121 | AAAGTGTAAA GCCTGGGGTG CCTAATGAGT GAGCTAACTC ACATTAATTG CGTTGCGCTC |
| | TTTCACATTT CGGACCCCAC GGATTACTCA CTCGATTGAG TGTAATTAAC GCAACGCGAG |
| 181 | ACTGCCCGCT TTCCAGTCGG GAAACCTGTC GTGCCAGCTG CATTAATGAA TCGGCCAACG |
| | TGACGGGCGA AAGGTCAGCC CTTTGGACAG CACGGTCGAC GTAATTACTT AGCCGGTTGC |
| 241 | CGCGGGGAGA GGCGGTTTGC GTATTGGGCG CTCTTCCGCT TCCTCGCTCA CTGACTCGCT |
| | GCGCCCCTCT CCGCCAAACG CATAACCCGC GAGAAGGCGA AGGAGCGAGT GACTGAGCGA |
| 301 | GCGCTCGGTC GTTCGGCTGC GGCGAGCGGT ATCAGCTCAC TCAAAGGCGG TAATACGGTT |
| | CGCGAGCCAG CAAGCCGACG CCGCTCGCCA TAGTCGAGTG AGTTTCCGCC ATTATGCCAA |
| 361 | ATCCACAGAA TCAGGGGATA ACGCAGGAAA GAACATGTGA GCAAAAGGCC AGCAAAAGGC |
| | TAGGTGTCTT AGTCCCCTAT TGCGTCCTTT CTTGTACACT CGTTTTCCGG TCGTTTTCCG |
| 421 | CAGGAACCGT AAAAAGGCCG CGTTGCTGGC GTTTTTCCAT AGGCTCCGCC CCCCTGACGA |
| | GTCCTTGGCA TTTTTCCGGC GCAACGACCG CAAAAAGGTA TCCGAGGCGG GGGGACTGCT |
| 481 | GCATCACAAA AATCGACGCT CAAGTCAGAG GTGGCGAAAC CCGACAGGAC TATAAAGATA |
| | CGTAGTGTTT TTAGCTGCGA GTTCAGTCTC CACCGCTTTG GGCTGTCCTG ATATTTCTAT |
| 541 | CCAGGCGTTT CCCCCTGGAA GCTCCCTCGT GCGCTCTCCT GTTCCGACCC TGCCGCTTAC |
| | GGTCCGCAAA GGGGGACCTT CGAGGGAGCA CGCGAGAGGA CAAGGCTGGG ACGGCGAATG |
| 601 | CGGATACCTG TCCGCCTTTC TCCCTTCGGG AAGCGTGGCG CTTTCTCATA GCTCACGCTG |
| | GCCTATGGAC AGGCGGAAAG AGGGAAGCCC TTCGCACCGC GAAAGAGTAT CGAGTGCGAC |
| | ApaLI |
| | ˜˜˜˜˜˜˜ |
| 661 | TAGGTATCTC AGTTCGGTGT AGGTCGTTCG CTCCAAGCTG GGCTGTGTGC ACGAACCCCC |
| | ATCCATAGAG TCAAGCCACA TCCAGCAAGC GAGGTTCGAC CCGACACACG TGCTTGGGGG |
| 721 | CGTTCAGCCC GACCGCTGCG CCTTATCCGG TAACTATCGT CTTGAGTCCA ACCCGGTAAG |
| | GCAAGTCGGG CTGGCGACGC GGAATAGGCC ATTGATAGCA GAACTCAGGT TGGGCCATTC |
| 781 | ACACGACTTA TCGCCACTGG CAGCAGCCAC TGGTAACAGG ATTAGCAGAG CGAGGTATGT |
| | TGTGCTGAAT AGCGGTGACC GTCGTCGGTG ACCATTGTCC TAATCGTCTC GCTCCATACA |
| 841 | AGGCGGTGCT ACAGAGTTCT TGAAGTGGTG GCCTAACTAC GGCTACACTA GAAGGACAGT |
| | TCCGCCACGA TGTCTCAAGA ACTTCACCAC CGGATTGATG CCGATGTGAT CTTCCTGTCA |
| 901 | ATTTGGTATC TGCGCTCTGC TGAAGCCAGT TACCTTCGGA AAAAGAGTTG GTAGCTCTTG |
| | TAAACCATAG ACGCGAGACG ACTTCGGTCA ATGGAAGCCT TTTTCTCAAC CATCGAGAAC |
| 961 | ATCCGGCAAA CAAACCACCG CTGGTAGCGG TGGTTTTTTT GTTTGCAAGC AGCAGATTAC |
| | TAGGCCGTTT GTTTGGTGGC GACCATCGCC ACCAAAAAAA CAAACGTTCG TCGTCTAATG |
| 1021 | GCGCAGAAAA AAAGGATCTC AAGAAGATCC TTTGATCTTT TCTACGGGGT CTGACGCTCA |
| | CGCGTCTTTT TTTCCTAGAG TTCTTCTAGG AAACTAGAAA AGATGCCCCA GACTGCGAGT |
| 1081 | GTGGAACGAA AACTCACGTT AAGGGATTTT GGTCATGAGA TTATCAAAAA GGATCTTCAC |
| | CACCTTGCTT TTGAGTGCAA TTCCCTAAAA CCAGTACTCT AATAGTTTTT CCTAGAAGTG |
| 1141 | CTAGATCCTT TTAAATTAAA AATGAAGTTT TAAATCAATC TAAAGTATAT ATGAGTAAAC |
| | GATCTAGGAA AATTTAATTT TTACTTCAAA ATTTAGTTAG ATTTCATATA TACTCATTTG |
| 1201 | TTGGTCTGAC AGTTACCAAT GCTTAATCAG TGAGGCACCT ATCTCAGCGA TCTGTCTATT |
| | AACCAGACTG TCAATGGTTA CGAATTAGTC ACTCCGTGGA TAGAGTCGCT AGACAGATAA |
| 1261 | TCGTTCATCC ATAGTTGCCT GACTCCCCGT CGTGTAGATA ACTACGATAC GGGAGGGCTT |
| | AGCAAGTAGG TATCAACGGA CTGAGGGGCA GCACATCTAT TGATGCTATG CCCTCCCGAA |
| 1321 | ACCATCTGGC CCCAGTGCTG CAATGATACC GCGAGACCCA CGCTCACCGG CTCCAGATTT |
| | TGGTAGACCG GGGTCACGAC GTTACTATGG CGCTCTGGGT GCGAGTGGCC GAGGTCTAAA |
| 1381 | ATCAGCAATA AACCAGCCAG CCGGAAGGGC CGAGCGCAGA AGTGGTCCTG CAACTTTATC |
| | TAGTCGTTAT TTGGTCGGTC GGCCTTCCCG GCTCGCGTCT TCACCAGGAC GTTGAAATAG |
| 1441 | CGCCTCCATC CAGTCTATTA ATTGTTGCCG GGAAGCTAGA GTAAGTAGTT CGCCAGTTAA |
| | GCGGAGGTAG GTCAGATAAT TAACAACGGC CCTTCGATCT CATTCATCAA GCGGTCAATT |
| 1501 | TAGTTTGCGC AACGTTGTTG CCATTGCTAC AGGCATCGTG GTGTCACGCT CGTCGTTTGG |
| | ATCAAACGCG TTGCAACAAC GGTAACGATG TCCGTAGCAC CACAGTGCGA GCAGCAAACC |
| 1561 | TATGGCTTCA TTCAGCTCCG GTTCCCAACG ATCAAGGCGA GTTACATGAT CCCCCATGTT |
| | ATACCGAAGT AAGTCGAGGC CAAGGGTTGC TAGTTCCGCT CAATGTACTA GGGGGTACAA |
| 1621 | GTGCAAAAAA GCGGTTAGCT CCTTCGGTCC TCCGATCGTT GTCAGAAGTA AGTTGGCCGC |
| | CACGTTTTTT CGCCAATCGA GGAAGCCAGG AGGCTAGCAA CAGTCTTCAT TCAACCGGCG |
| 1681 | AGTGTTATCA CTCATGGTTA TGGCAGCACT GCATAATTCT CTTACTGTCA TGCCATCCGT |
| | TCACAATAGT GAGTACCAAT ACCGTCGTGA CGTATTAAGA GAATGACAGT ACGGTAGGCA |
| 1741 | AAGATGCTTT TCTGTGACTG GTGAGTACTC AACCAAGTCA TTCTGAGAAT AGTGTATGCG |
| | TTCTACGAAA AGACACTGAC CACTCATGAG TTGGTTCAGT AAGACTCTTA TCACATACGC |
| 1801 | GCGACCGAGT TGCTCTTGCC CGGCGTCAAT ACGGGATAAT ACCGCGCCAC ATAGCAGAAC |
| | CGCTGGCTCA ACGAGAACGG GCCGCAGTTA TGCCCTATTA TGGCGCGGTG TATCGTCTTG |
| 1861 | TTTAAAAGTG CTCATCATTG GAAAACGTTC TTCGGGGCGA AAACTCTCAA GGATCTTACC |
| | AAATTTTCAC GAGTAGTAAC CTTTTGCAAG AAGCCCCGCT TTTGAGAGTT CCTAGAATGG |
| | ApaLI |
| | ˜˜˜˜˜˜ |
| 1921 | GCTGTTGAGA TCCAGTTCGA TGTAACCCAC TCGTGCACCC AACTGATCTT CAGCATCTTT |
| | CGACAACTCT AGGTCAAGCT ACATTGGGTG AGCACGTGGG TTGACTAGAA GTCGTAGAAA |
| 1981 | TACTTTCACC AGCGTTTCTG GGTGAGCAAA AACAGGAAGG CAAAATGCCG CAAAAAAGGG |
| | ATGAAAGTGG TCGCAAAGAC CCACTCGTTT TTGTCCTTCC GTTTTACGGC GTTTTTTCCC |
| 2041 | AATAAGGGCG ACACGGAAAT GTTGAATACT CATACTCTTC CTTTTTCAAT ATTATTGAAG |
| | TTATTCCCGC TGTGCCTTTA CAACTTATGA GTATGAGAAG GAAAAAGTTA TAATAACTTC |
| 2101 | CATTTATCAG GGTTATTGTC TCATGAGCGG ATACATATTT GAATGTATTT AGAAAAATAA |
| | GTAAATAGTC CCAATAACAG AGTACTCGCC TATGTATAAA CTTACATAAA TCTTTTTATT |
| 2161 | ACAAATAGGG GTTCCGCGCA CATTTCCCCG AAAAGTGCCA CCTGGGAAAT TGTAAACGTT |
| | TGTTTATCCC CAAGGCGCGT GTAAAGGGGC TTTTCACGGT GGACCCTTTA ACATTTGCAA |
| 2221 | AATATTTTGT TAAAATTCGC GTTAAATTTT TGTTAAATCA GCTCATTTTT TAACCAATAG |
| | TTATAAAACA ATTTTAAGCG CAATTTAAAA ACAATTTAGT CGAGTAAAAA ATTGGTTATC |
| 2281 | GCCGAAATCG GCAAAATCCC TTATAAATCA AAAGAATAGA CCGAGATAGG GTTGAGTGTT |
| | CGGCTTTAGC CGTTTTAGGG AATATTTAGT TTTCTTATCT GGCTCTATCC CAACTCACAA |
| 2341 | GTTCCAGTTT GGAACAAGAG TCCACTATTA AAGAACGTGG ACTCCAACGT CAAAGGGCGA |
| | CAAGGTCAAA CCTTGTTCTC AGGTGATAAT TTCTTGCACC TGAGGTTGCA GTTTCCCGCT |
| 2401 | AAAACCGTCT ATCAGGGCGA TGGCCCACTA CGTGAACCAT CACCCTAATC AAGTTTTTTG |
| | TTTTGGCAGA TAGTCCCGCT ACCGGGTGAT GCACTTGGTA GTGGGATTAG TTCAAAAAAC |
| 2461 | GGGTCGAGGT GCCGTAAAGC ACTAAATCGG AACCCTAAAG GGAGCCCCCG ATTTAGAGCT |
| | CCCAGCTCCA CGGCATTTCG TGATTTAGCC TTGGGATTTC CCTCGGGGGC TAAATCTCGA |
| 2521 | TGACGGGGAA AGCCGGCGAA CGTGGCGAGA AAGGAAGGGA AGAAAGCGAA AGGAGCGGGC |
| | ACTGCCCCTT TCGGCCGCTT GCACCGCTCT TTCCTTCCCT TCTTTCGCTT TCCTCGCCCG |
| 2581 | GCTAGGGCGC TGGCAAGTGT AGCGGTCACG CTGCGCGTAA CCACCACACC CGCCGCGCTT |
| | CGATCCCGCG ACCGTTCACA TCGCCAGTGC GACGCGCATT GGTGGTGTGG GCGGCGCGAA |
| 2641 | AATGCGCCGC TACAGGGCGC GTCGCGCCAT TCGCCATTCA GGCTGCGCAA CTGTTGGGAA |
| | TTACGCGGCG ATGTCCCGCG CAGCGCGGTA AGCGGTAAGT CCGACGCGTT GACAACCCTT |
| 2701 | GGGCGATCGG TGCGGGCCTC TTCGCTATTA CGCCAGCTGG CGAAAGGGGG ATGTGCTGCA |
| | CCCGCTAGCC ACGCCCGGAG AAGCGATAAT GCGGTCGACC GCTTTCCCCC TACACGACGT |
| 2761 | AGGCGATTAA GTTGGGTAAC GCCAGGGTTT TCCCAGTCAC GACGTTGTAA AACGACGGCC |
| | TCCGCTAATT CAACCCATTG CGGTCCCAAA AGGGTCAGTG CTGCAACATT TTGCTGCCGG |
| | ClaI |
| | ˜˜˜˜˜ |
| 2821 | AGTGAATTGT AATACGACTC ACTATAGGGC GAATTGGGTA CCGGGCCTCG ACGGTATCGA |
| | TCACTTAACA TTATGCTGAG TGATATCCCG CTTAACCCAT GGCCCGGAGC TGCCATAGCT |
| | ClaI |
| | ˜ |
| 2881 | TTGCAGGTCG ATATTTAAAA AAAATTATAA TAATGTTAAA GTTGCTTCAT ACGTTGAAGT |
| | AACGTCCAGC TATAAATTTT TTTTAATATT ATTACAATTT CAACGAAGTA TGCAACTTCA |
| 2941 | ACCTTACACA ACAAATAATG CAGACGTCGA AATGACTGAC ATAACAAATG CGCCCTTTCG |
| | TGGAATGTGT TGTTTATTAC GTCTGCAGCT TTACTGACTG TATTGTTTAC GCGGGAAAGC |
| 3001 | CCCAAAATCT AAAGCAAGGA GAAGATTAGA TTTTACCAAC ATGGCGCCGC AGCCGTGTTG |
| | GGGTTTTAGA TTTCGTTCCT CTTCTAATCT AAAATGGTTG TACCGCGGCG TCGGCACAAC |
| 3061 | TATTGACGAC GGCAATTTTG CAAAACTTTA CTGTGATATA TTTATTAAAT TAAGTTTGCT |
| | ATAACTGCTG CCGTTAAAAC GTTTTGAAAT GACACTATAT AAATAATTTA ATTCAAACGA |
| 3121 | TTAAAATGAG TTTTTTTACA AATCTTCGCA GAGTCAATAA ATTGTATCCT AATCAGGCCA |
| | AATTTTACTC AAAAAAATGT TTAGAAGCGT CTCAGTTATT TAACATAGGA TTAGTCCGGT |
| 3181 | GTTTTCTTGC TGATAATACG CGTCTTTTAA CAAGGCACTC CCGCCGGTTT CACAAATGTG |
| | CAAAAGAACG ACTATTATGC GCAGAAAATT GTTCCGTGAG GGCGGCCAAA GTGTTTACAC |
| 3241 | CTCAACGCGC CCAGTGTACG CAACCTTGGA AACAACAGAT ATGGGCCGGG CTATCAATTA |
| | GAGTTGCGCG GGTCACATGC GTTGGAACCT TTGTTGTCTA TACCCGGCCC GATAGTTAAT |
| 3301 | TCTAACAACC GGTTTGTGAG CACTTCAGAC ATAAACAGAA TTACTCGTAA CAACGATGTC |
| | AGATTGTTGG CCAAACACTC GTGAAGTCTG TATTTGTCTT AATGAGCATT GTTGCTACAG |
| 3361 | CCCAACATAC GCGGAGTATT TCAGGGCATT TCAGACCCTC AAATAAACTC ATTGAGCCAA |
| | GGGTTGTATG CGCCTCATAA AGTCCCGTAA AGTCTGGGAG TTTATTTGAG TAACTCGGTT |
| 3421 | TTGCGGCGCA TGGACCAACG TGCCAGACTT TCATTACCAC ACCAAACAGA CGCGATCCAA |
| | AACGCCGCGT ACCTGGTTGC ACGGTCTGAA AGTAATGGTG TGGTTTGTCT GCGCTAGGTT |
| 3481 | TGCAGTCAGA CAAAACTTCC CGGAGACCAA CGTGCGCACG CCCGAAGGTG TTCAAAATGC |
| | ACGTCAGTCT GTTTTGAAGG GCCTCTGGTT GCACGCGTGC GGGCTTCCAC AAGTTTTACG |
| | PstI |
| | ˜˜˜˜˜˜ |
| 3541 | ACTGCAGCAA AACCCCCCCG TTTACATAAT CACATGAGAA CCTTGAAAGT AGCAGGAGTG |
| | TGACGTCGTT TTGGGGGGGC AAATGTATTA GTGTACTCTT GGAACTTTCA TCGTCCTCAC |
| 3601 | GGCATACTCT TGGGCCGGCG GCGGTTATCT TTTGTTTACC GCCGCCACAT TAGTACAAGA |
| | CCGTATGAGA ACCCGGCCGC CGCCAATAGA AAACAAATGG CGGCGGTGTA ATCATGTTCT |
| 3661 | TATAATCAAC GCCATCAATA GAACCGGCGG AAGTTATTAT GTGCAAGGTA GAAACGCCGG |
| | ATATTAGTTG CGGTAGTTAT CTTGGCCGCC TTCAATAATA CACGTTCCAT CTTTGCGGCC |
| 3721 | AGAAAACGCC GAGGCCTGTT TGTTATTGCA GCGCACTTGT CGTCAAGACC GCAATCTAGC |
| | TCTTTTGCGG CTCCGGACAA ACAATAACGT CGCGTGAACA GCAGTTCTGG CGTTAGATCG |
| 3781 | TCAGTCGGAT GTTAACATTT GCTCAAGAGA CCCCTTGTTG GCTAACGATT CGCCCCTACT |
| | AGTCAGCCTA CAATTGTAAA CGAGTTCTCT GGGGAACAAC CGATTGCTAA GCGGGGATGA |
| 3841 | AACCAACATG TGCCAAGGAT TTAACTATGA AACAGAAAAA ACAGTTTGTC GCGGCAGCAA |
| | TTGGTTGTAC ACGGTTCCTA AATTGATACT TTGTCTTTTT TGTCAAACAG CGCCGTCGTT |
| 3901 | TCCGGCCGCT AACCCAACTT CGCCTCAATA CGTAGATATT AGCGATCTTC TGCGGGCCAA |
| | AGGCCGGCGA TTGGGTTGAA GCGGAGTTAT GCATCTATAA TCGCTAGAAG ACGCCCGGTT |
| 3961 | ACAATCATGT GCATCGAACC TTACACGTTT AGTGATTTAA TTGGCGACTT GCGTTTACAT |
| | TGTTAGTACA CGTAGCTTGG AATGTGCAAA TCACTAAATT AACCGCTGAA CGCAAATGTA |
| 4021 | TGGTTACTGG GAAGAGAAGG TTTAATCGGC AAATCGTCCA ACGGTAGTGA CAGCATCCGC |
| | ACCAATGACC CTTCTCTTCC AAATTAGCCG TTTAGCAGGT TGCCATCACT GTCGTAGGCG |
| 4081 | AACAAAATAA TGCCTCATCA TTATGATGAT AGGCGCGTTC TTGTTTTTAG GTTTAATACT |
| | TTGTTTTATT ACGGAGTAGT AATACTACTA TCCGCGCAAG AACAAAAATC CAAATTATGA |
| 4141 | TTATTTTATC TACAGATACA TGACAAAAGG AGGAGGAGGA GGAGGAAGCG GTGGGGCACC |
| | AATAAAATAG ATGTCTATGT ACTGTTTTCC TCCTCCTCCT CCTCCTTCGC CACCCCGTGG |
| 4201 | AACTCCCATT GTTGTTATTA TGCAACACCC CACATCAACA GCGGCCCCTC GTCGATAATA |
| | TTGAGGGTAA CAACAATAAT ACGTTGTGGG GTGTAGTTGT CGCCGGGGAG CAGCTATTAT |
| 4261 | AAAGACAAAA ATAATATAAA ATATATGTAT AATTAATTAA ATTCAAAAGA TATGTATAAT |
| | TTTCTGTTTT TATTATATTT TATATACATA TTAATTAATT TAAGTTTTCT ATACATATTA |
| 4321 | TAATTAAATT CAAATTTTTT ATATTTACAA TTTAGTTTTT GTTCCGCAAA CGTTATAGCG |
| | ATTAATTTAA GTTTAAAAAA TATAAATGTT AAATCAAAAA CAAGGCGTTT GCAATATCGC |
| 4381 | TCGGACAACG GAACCAGACC CTGTAATATT AAAGCTAACA ATTTTAACAA ATTATTGTGC |
| | AGCCTGTTGC CTTGGTCTGG GACATTATAA TTTCGATTGT TAAAATTGTT TAATAACACG |
| 4441 | AATGTAGTGC TCTCTCTTCG GTTCACTTTA CTGATTACAA ACATGTGATG CTTAAATCTA |
| | TTACATCACG AGAGAGAAGC CAAGTGAAAT GACTAATGTT TGTACACTAC GAATTTAGAT |
| 4501 | TTATATTTTT GAATTACTTG ACTAGCGTCT ACATCTTTAA TCTCGCCAGA AATCCAATAA |
| | AATATAAAAA CTTAATGAAC TGATCGCAGA TGTAGAAATT AGAGCGGTCT TTAGGTTATT |
| 4561 | AACTCTTCGT TTTTCTTAGC TATAGTCAAC CGCTCTTCGT TTTTGAAAGA CAATACTATA |
| | TTGAGAAGCA AAAAGAATCG ATATCAGTTG GCGAGAAGCA AAAACTTTCT GTTATGATAT |
| 4621 | AAATTGTGAC CTTTTACATT ATCCACATTC TGAGTCAAAT ACTGTTCGAC AATGTGCATG |
| | TTTAACACTG GAAAATGTAA TAGGTGTAAG ACTCAGTTTA TGACAAGCTG TTACACGTAC |
| 4681 | CTGCCGTCCT CCTTCTTAAC CTTTTTTAAA TTTTCAGCGT TATTATTACT CGCAATATTG |
| | GACGGCAGGA GGAAGAATTG GAAAAAATTT AAAAGTCGCA ATAATAATGA GCGTTATAAC |
| 4741 | TCATGATATT TATAATTATT AAACAAAAGA TTAGCGACAC TACTGTATTT GTACGTGAGC |
| | AGTACTATAA ATATTAATAA TTTGTTTTCT AATCGCTGTG ATGACATAAA CATGCACTCG |
| 4801 | GTACTTTTTT TGTTAACAAT TAAATTTAAA TTGTCCACCA CATATTTGTT TGGGGGATTG |
| | CATGAAAAAA ACAATTGTTA ATTTAAATTT AACAGGTGGT GTATAAACAA ACCCCCTAAC |
| 4861 | TCGGGAAACT TTACACTTTC CGAATACTTT AATATTTGAC TCACATACGG CGATACAAAA |
| | AGCCCTTTGA AATGTGAAAG GCTTATGAAA TTATAAACTG AGTGTATGCC GCTATGTTTT |
| 4921 | AAATTATTAG ATGCAGTCTC AATTTCATTA CTCTCTTTAC GACTAAGCAT AATAGGCAAA |
| | TTTAATAATC TACGTCAGAG TTAAAGTAAT GAGAGAAATG CTGATTCGTA TTATCCGTTT |
| 4981 | GTAAATAAAT TTTTATCTTG ATACATTTCG TACAACTTGC TCAAAAGAAA CCCACACTTT |
| | CATTTATTTA AAAATAGAAC TATGTAAAGC ATGTTGAACG AGTTTTCTTT GGGTGTGAAA |
| 5041 | CTTTCGCCCA ACGATTGTAA CAAAGTCACA AATGTGGTTT GCGCGTAATA CATATCTAAA |
| | GAAAGCGGGT TGCTAACATT GTTTCAGTGT TTACACCAAA CGCGCATTAT GTATAGATTT |
| 5101 | TTAAAATATG AAGTCAGAGC AGCTTTAAAC GTGTGATGCA CATCGACAAA GTGGCATTTT |
| | AATTTTATAC TTCAGTCTCG TCGAAATTTG CACACTACGT GTAGCTGTTT CACCGTAAAA |
| 5161 | TTACAATTTT GTGCAGCCGT CTCGTCGTTG CACACATCTT GAGAATGAGG AATTTCTATG |
| | AATGTTAAAA CACGTCGGCA GAGCAGCAAC GTGTGTAGAA CTCTTACTCC TTAAAGATAC |
| 5221 | CCGGTTTCTT TAACCAAATT GTACGAGATC ATAAATCTAA TTTTATCAAA AGTTACCACA |
| | GGCCAAAGAA ATTGGTTTAA CATGCTCTAG TATTTAGATT AAAATAGTTT TCAATGGTGT |
| 5281 | AACACGCGAT TATCTACCAT GTAATAGTTG TTTGTATATT CGTACACCAC ATTGCTCACG |
| | TTGTGCGCTA ATAGATGGTA CATTATCAAC AAACATATAA GCATGTGGTG TAACGAGTGC |
| 5341 | TACTTGGCAA ATATAATTTC AAACGGCTTT ACTTCACTTT TTTTAACCAC AAACATGTAA |
| | ATGAACCGTT TATATTAAAG TTTGCCGAAA TGAAGTGAAA AAAATTGGTG TTTGTACATT |
| 5401 | TAACCAGTTT CGGACATATG GTCGGAGAAC CTATTGGAAT TGTAGTCGTT GTCGTCGAAA |
| | ATTGGTCAAA GCCTGTATAC CAGCCTCTTG GATAACCTTA ACATCAGCAA CAGCAGCTTT |
| 5461 | CGCATCAAAT ACGGCGCAAA ATCATTAGTA AAATAATGCG TAATTTCTTG AGTTGAAGCA |
| | GCGTAGTTTA TGCCGCGTTT TAGTAATCAT TTTATTACGC ATTAAAGAAC TCAACTTCGT |
| 5521 | ACCGTGCAAA TGTTCGTGTT GTGATTAATT GTCTGCTCAA GGGTTGCACA GCTTTGAATT |
| | TGGCACGTTT ACAAGCACAA CACTAATTAA CAGACGAGTT CCCAACGTGT CGAAACTTAA |
| 5581 | GTGCTTTTCT TGTATTTAGG CTTCAATTTA TTCTTGTTAA ATTGGCCCAC CACACTTTGT |
| | CACGAAAAGA ACATAAATCC GAAGTTAAAT AAGAACAATT TAACCGGGTG GTGTGAAACA |
| 5641 | GAATCGTCCA AGTATTCGTC CAGCTTCCGT TTAGTTCCAG TTGCCGATGG TTGGTTCACA |
| | CTTAGCAGGT TCATAAGCAG GTCGAAGGCA AATCAAGGTC AACGGCTACC AACCAAGTGT |
| 5701 | CCAACAGGAT GCTCAAAAGA TTCCGCATTA TAAGCAGAAC TGGGCGATGG TTGCTCCGCA |
| | GGTTGTCCTA CGAGTTTTCT AAGGCGTAAT ATTCGTCTTG ACCCGCTACC AACGAGGCGT |
| 5761 | ACAGGCAGCT CAAAAGATTC CGCATTATAA GCAGAACTAA CTGCTTCTCC GAGATTATCA |
| | TGTCCGTCGA GTTTTCTAAG GCGTAATATT CGTCTTGATT GACGAAGAGG CTCTAATAGT |
| 5821 | GTGGTCTTGA GCAAACATTC CATTATATCG TTATCATCAG TTAACGAATT GACGCTTGCC |
| | CACCAGAACT CGTTTGTAAG GTAATATAGC AATAGTAGTC AATTGCTTAA CTGCGAACGG |
| | PstI |
| | ˜˜˜˜˜˜˜ |
| 5881 | AAAAAGTTTG AAGCTGCCTG CAGTCTGCTG TCAGATACTA CCGTGTCGGC TCCATCCGGC |
| | TTTTTCAAAC TTCGACGGAC GTCAGACGAC AGTCTATGAT GGCACAGCCG AGGTAGGCCG |
| 5941 | GTGGGATTGT TATAATAATT CAAATAGTCG TTGGGCTGTT GTTTATCACA AAACTCTGAA |
| | CACCCTAACA ATATTATTAA GTTTATCAGC AACCCGACAA CAAATAGTGT TTTGAGACTT |
| | AvaI |
| | ˜˜˜˜˜˜˜ |
| 6001 | TAGCCGTTGT CGAACGACGC TCGGGACGGC GTCGGAGCAC TGGTGTACGA CGCGTTAAAA |
| | ATCGGCAACA GCTTGCTGCG AGCCCTGCCG CAGCCTCGTG ACCACATGCT GCGCAATTTT |
| 6061 | TTAATTTGCG TCATAGTCGT TTGGTTGTTC ACGATCGTGT CCCGCCAATG TCAACTTGCA |
| | AATTAAACGC AGTATCAGCA AACCAACAAG TGCTAGCACA GGGCGGTTAC AGTTGAACGT |
| 6121 | ACTGAAACAA TATTCAACAT GAACGTCAAT TTATACTGCC CTAATGGCGA ACACGATAAT |
| | TGACTTTGTT ATAAGTTGTA CTTGCAGTTA AATATGACGG GATTACCGCT TGTGCTATTA |
| 6181 | AATATTTTTT TTATTATGCC CTCTAAAACC AATGCGGTTA TCGTTTATTT ATTCAAATTA |
| | TTATAAAAAA AATAATACGG GAGATTTTGG TTACGCCAAT AGCAAATAAA TAAGTTTAAT |
| 6241 | GATACAGAAC ATCCGCCGAC ATACAATGTT AATGCAAAAA CTCGTTTGGT GAGCGGATAC |
| | CTATGTCTTG TAGGCGGCTG TATGTTACAA TTACGTTTTT GAGCAAACCA CTCGCCTATG |
| 6301 | GAAAACAGTC GGCCGATAAA CATTAATCTG AGGTCGATAA CACCGTCCTT GAACGGAACA |
| | CTTTTGTCAG CCGGCTATTT GTAATTAGAC TCCAGCTATT GTGGCAGGAA CTTGCCTTGT |
| 6361 | CGAGGAGCGT ACGTGATCAG CTGCATTCGC GCGCCGCGCC TTTATCGAGA TTTATTTACA |
| | GCTCCTCGCA TGCACTAGTC GACGTAAGCG CGCGGCGCGG AAATAGCTCT AAATAAATGT |
| 6421 | TACAACAAGT ACACTGCGCC GTTGGCATTT GTGGTAACGC GCACACAAGC AGAGCTGCAA |
| | ATGTTGTTCA TGTGACGCGG CAACCGTAAA CACCATTGCG CGTGTGTTCG TCTCGACGTT |
| 6481 | GTGTGGCACA TTTTGTCTGT GCGCAAAACC TTTGAAGCCA AAAGCACAAG GTCCGTTACG |
| | CACACCGTGT AAAACAGACA CGCGTTTTGG AAACTTCGGT TTTCGTGTTC CAGGCAATGC |
| 6541 | GGCATGCTAG CGCACACGGA CAACGGACCC GACAAATTCT ACGCCAAGGA TTTAATGATA |
| | CCGTACGATC GCGTGTGCCT GTTGCCTGGG CTGTTTAAGA TGCGGTTCCT AAATTACTAT |
| 6601 | ATGTCGGGCA ACGTGTCGGT GCATTTTATT AATAACTTAC AAAATGTCGC GCGCATCACA |
| | TACAGCCCGT TGCACAGCCA CGTAAAATAA TTATTGAATG TTTTACAGCG CGCGTAGTGT |
| | HindIII EcoRI |
| | ˜˜˜˜˜˜˜ ˜˜˜ |
| 6661 | AAGACATTGA TATATTTAAA CATTTATGTC CCGAACTGCA ACGATAAGCT TGATATCGAA |
| | TTCTGTAACT ATATAAATTT GTAAATACAG GGCTTGACGT TGCTATTCGA ACTATAGCTT |
| | PstI |
| | ˜˜˜˜˜˜ |
| | EcoRI |
| | ˜˜˜ |
| 6721 | TTCCTGCAGC CCAATATTAC GTTCGTGCCA GAAATTAATT TCTCCGCGTC GTATTATACG |
| | AAGGACGTCG GGTTATAATG CAAGCACGGT CTTTAATTAA AGAGGCGCAG CATAATATGC |
| 6781 | ATTTATACGG TACAGCAGCT TGGCCCACAA ATAGATCGTT TTATGATTTT GATGATGGAG |
| | TAAATATGCC ATGTCGTCGA ACCGGGTGTT TATCTAGCAA AATACTAAAA CTACTACCTC |
| 6841 | GTGCGCTCAA GATGAAACCC ATTCAGACGT TATTAGTTGC GTCAAGTATT TGGCAATTTG |
| | CACGCGAGTT CTACTTTGGG TAAGTCTGCA ATAATCAACG CAGTTCATAA ACCGTTAAAC |
| 6901 | CTACGACGCA ATTATTGTGG AAGAAGCGTA ATTTGTGAAC AGCCCATTCG AGGCTAGATT |
| | GATGCTGCGT TAATAACACC TTCTTCGCAT TAAACACTTG TCGGGTAAGC TCCGATCTAA |
| 6961 | GAAAAAGTAT ATTGATATTA AATCATATAA ATTGTTTATG AGGCCTTCAA ACGAATCTTG |
| | CTTTTTCATA TAACTATAAT TTAGTATATT TAACAAATAC TCCGGAAGTT TGCTTAGAAC |
| 7021 | TAAAGATTAT TTATTAAAAT TGTTCAACGA TTGTATGAGA GGGTCATTTG TTTTTCAAAA |
| | ATTTCTAATA AATAATTTTA ACAAGTTGCT AACATACTCT CCCAGTAAAC AAAAAGTTTT |
| | EcoRI |
| | ˜˜˜˜˜˜ |
| 7081 | CTGAACTCGC TTTACGAGTA GAATTCTACT TGTAAAACAC AATCAAGAGA TGATGTCATT |
| | GACTTGAGCG AAATGCTCAT CTTAAGATGA ACATTTTGTG TTAGTTCTCT ACTACAGTAA |
| 7141 | TGTTTTTCAA AACTGAATGA TGTCATTTGT TTTTTAAAAC TAAACTCGCT TTTACGAGTA |
| | ACAAAAAGTT TTGACTTACT ACAGTAAACA AAAAATTTTG ATTTGAGCGA AAATGCTCAT |
| | EcoRI |
| | ˜˜˜˜˜˜ |
| 7201 | GAATTCTACG TGTAAAACAT AATCAAGAGA TGATGTCATT TGTTTTTCAA AACTGAACCG |
| | CTTAAGATGC ACATTTTGTA TTAGTTCTCT ACTACAGTAA ACAAAAAGTT TTGACTTGGC |
| | EcoRI |
| | ˜˜˜˜˜˜ |
| 7261 | GCTTTACGAG TAGAATTCTA CGTGTAAAAC ATAATCAAGA GATGATGTCA TCATTAAACT |
| | CGAAATGCTC ATCTTAAGAT GCACATTTTG TATTAGTTCT CTACTACAGT AGTAATTTGA |
| 7321 | GATGTCATTT TTATACACGA TTGTTAACAT GTTTAATAAT GACTAATTTG TTTTTCAAAT |
| | CTACAGTAAA AATATGTGCT AACAATTGTA CAAATTATTA CTGATTAAAC AAAAAGTTTA |
| | EcoRI |
| | ˜˜˜˜˜˜˜ |
| 7381 | TAAACTCGCT TTACAAGTAG AATTCTACTT GTAACGCACG ATTAAAATTA TTATAATCAG |
| | ATTTGAGCGA AATGTTCATC TTAAGATGAA CATTGCGTGC TAATTTTAAT AATATTAGTC |
| 7441 | GAATGATGTC ATTTGTTTTC GTCATAAAAT GTTTATACAA CGGAATCTTC TTGTAAATTA |
| | CTTACTACAG TAAACAAAAG CAGTATTTTA CAAATATGTT GCCTTAGAAG AACATTTAAT |
| 7501 | TCCAAATAAT ATAATTTATC CGATTCTACG TTACATTTAA ATTCGTTGTT ATCGTACAAT |
| | AGGTTTATTA TATTAAATAG GCTAAGATGC AATGTAAATT TAAGCAACAA TAGCATGTTA |
| 7561 | TCTTCAGGAC ACGCCATGTA TTGGCCGTTT TTAACGTGCA ACCAACGATT GTATTTGACG |
| | AGAAGTCCTG TGCGGTACAT AACCGGCAAA AATTGCACGT TGGTTGCTAA CATAAACTGC |
| 7621 | CCGTCGTTGG ATTGCGTGTT CAGGTTGGCG TACACGTGAC TGGGCACGGC TTCTTTTTTT |
| | GGCAGCAACC TAACGCACAA GTCCAACCGC ATGTGCACTG ACCCGTGCCG AAGAAAAAAA |
| 7681 | ACCACTATCG CATCTTCGTC GTACGCGGAT CTACAACCAA TCCCGTTGCC CACATAAGCG |
| | TGGTGATAGC GTAGAAGCAG CATGCGCCTA GATGTTGGTT AGGGCAACGG GTGTATTCGC |
| 7741 | TACGCGTTTA AAACGTGCGA TAGGTCTTTG GCCAATTCGC AATCAGCGTC CACTTTAACG |
| | ATGCGCAAAT TTTGCACGCT ATCCAGAAAC CGGTTAAGCG TTAGTCGCAG GTGAAATTGC |
| 7801 | TTGTTGCGTA ACTCGTTTAA AGCATTAATA ATGACGTCAT TTTCCGCATG ACAACTGGTT |
| | AACAACGCAT TGAGCAAATT TCGTAATTAT TACTGCAGTA AAAGGCGTAC TGTTGACCAA |
| 7861 | AGCTTGAAAA ACGGAACCGA GTAGTGGCAT GAATAAAATA AATCTTTGTT GTCTAATATT |
| | TCGAACTTTT TGCCTTGGCT CATCACCGTA CTTATTTTAT TTAGAAACAA CAGATTATAA |
| 7921 | GGGGGGGAGC TCTTGTGAGT CCTCGCGGGT AGGTACCACC ACCCTGCCTA TTTCTGCCGT |
| | CCCCCCCTCG AGAACACTCA GGAGCGCCCA TCCATGGTGG TGGGACGGAT AAAGACGGCA |
| 7981 | GAAGCAGTAA TGCGTTTCGG TTTGAAGAGT GGGGCGGCCG TGGTACTGAG ACCTTAGAAC |
| | CTTCGTCATT ACGCAAAGCC AAACTTCTCA CCCCGCCGGC ACCATGACTC TGGAATCTTG |
| 8041 | TCATATCTGA AGGTGGGTGG CACATTTACG TTGTAGATGT CTATGGGCTC CAGTAACCAC |
| | AGTATAGACT TCCACCCACC GTGTAAATGC AACATCTACA GATACCCGAG GTCATTGGTG |
| 8101 | TTAACATCAG GTGGGCTGTG AGCTCTTACA CCCATCTACG CAATAAAAAA TTAAAAATAA |
| | AATTGTAGTC CACCCGACAC TCGAGAATGT GGGTAGATGC GTTATTTTTT AATTTTTATT |
| 8161 | ATATGTTTGA AGTCCGTAAC ATAGATTCCG TATTTTTACA GTTGTTTTTC ACGTTTTTCA |
| | TATACAAACT TCAGGCATTG TATCTAAGGC ATAAAAATGT CAACAAAAAG TGCAAAAAGT |
| 8221 | TTTCTTCACC GACAATGGAA AATAATCACA CACAAATACA CTGTATAGTA ACAACGAGCA |
| | AAAGAAGTGG CTGTTACCTT TTATTAGTGT GTGTTTATGT GACATATCAT TGTTGCTCGT |
| 8281 | GAGCCGATTT TGGAGTTTCG ATAAAGCGAG GCTACCAAGA ATGCGGCAGA TAAGATTTAC |
| | CTCGGCTAAA ACCTCAAAGC TATTTCGCTC CGATGGTTCT TACGCCGTCT ATTCTAAATG |
| 8341 | GTACATTCAA GAGTCGCTGA TAACAACTTT TACCTCTCAA ATTGCCCACA GTGCGATCAC |
| | CATGTAAGTT CTCAGCGACT ATTGTTGAAA ATGGAGAGTT TAACGGGTGT CACGCTAGTG |
| 8401 | AAGAAACATA GACGAACGGA TCTGTGCGCA ACGAGCCGCT ACGATATCAT TATCATACAG |
| | TTCTTTGTAT CTGCTTGCCT AGACACGCGT TGCTCGGCGA TGCTATAGTA ATAGTATGTC |
| 8461 | ATTTTTATCT TTTCATCTAG CTTCAGTTAG TGATGCTTTC TGATCTCTTC ATAATTATAA |
| | TAAAAATAGA AAAGTAGATC GAAGTCAATC ACTACGAAAG ACTAGAGAAG TATTAATATT |
| 8521 | TTAAAAAGAA TAAATTATCT AGTAATATAG TTCTACTACG GTACACGAAT TTTGAGATTA |
| | AATTTTTCTT ATTTAATAGA TCATTATATC AAGATGATGC CATGTGCTTA AAACTCTAAT |
| 8581 | ATTAACCGGA TTTTCTGGGT TATGATTTAC ATCGGTACAG AATCTAGTGA AAGCACGTCG |
| | TAATTGGCCT AAAAGACCCA ATACTAAATG TAGCCATGTC TTAGATCACT TTCGTGCAGC |
| 8641 | AGTGAAATTC TATGAAACTT CGGCGGGAGT CGGGGAGAGG TTACAAGCGA CCGCGAGGTG |
| | TCACTTTAAG ATACTTTGAA GCCGCCCTCA GCCCCTCTCC AATGTTCGCT GGCGCTCCAC |
| 8701 | CCGCTAACTT AATCAGTTAT CAAGGCATCG CCTTATCAAA AGATGCGAGC TGATAGCGTG |
| | GGCGATTGAA TTAGTCAATA GTTCCGTAGC GGAATAGTTT TCTACGCTCG ACTATCGCAC |
| 8761 | CGCGTTACCA TATATGGTGA CAAAAACTGA GTCAGCCCGC GATTGGTGGA AAAACAAACT |
| | GCGCAATGGT ATATACCACT GTTTTTGACT CAGTCGGGCG CTAACCACCT TTTTGTTTGA |
| 8821 | GGAGCCGATA CTGTGTAAAT TGTGATAACG GCTCTTTTAT ATAGTTTATC CTCACGAGTC |
| | CCTCGGCTAT GACACATTTA ACACTATTGC CGAGAAAATA TATCAAATAG GAGTGCTCAG |
| 8881 | GGTTCTCATT TACTAAGGTG TGCTCGAACA GTGCGCATTC GCATCTACGT ACTTGTCACT |
| | CCAAGAGTAA ATGATTCCAC ACGAGCTTGT CACGCGTAAG CGTAGATGCA TGAACAGTGA |
| 8941 | TATTTAATAA TACTATGTAA GTTTTAATTT TAAAATTGCG AAAGAAAAAA AAACATATTT |
| | ATAAATTATT ATGATACATT CAAAATTAAA ATTTTAACGC TTTCTTTTTT TTTGTATAAA |
| 9001 | ATTTATTTGT AAAATTTGAA TTTCGAAGGT TCTCCGTCCC TTTACCTTTA AGTATTACAT |
| | TAAATAAACA TTTTAAACTT AAAGCTTCCA AGAGGCAGGG AAATGGAAAT TCATAATGTA |
| 9061 | ATGTTTGAGT GTTTTTTTTT TTTAATAATA CGCTAATGAT AACGTGTTAC GTTACATAAT |
| | TACAAACTCA CAAAAAAAAA AAATTATTAT GCGATTACTA TTGCACAATG CAATGTATTA |
| 9121 | TGTTGCATAA CTAGTGAAGT GAAATTTTTT ATAAAAAAAA ACATTTTTCG GAATTTAGTG |
| | ACAACGTATT GATCACTTCA CTTTAAAAAA TATTTTTTTT TGTAAAAAGC CTTAAATCAC |
| | PstI |
| | ˜˜˜˜˜˜ |
| 9181 | TACTGCAGAT GTTAATAAAC ACTACTAAAT AAGAAATAAG TTTATTGGAC GCACATTTCA |
| | ATGACGTCTA CAATTATTTG TGATGATTTA TTCTTTATTC AAATAACCTG CGTGTAAAGT |
| | ClaI |
| | ˜˜˜˜˜˜˜ |
| 9241 | AAGTGTCCAC TCGCATCGAT CAATTCGGAA ACAGAAATTG GGAACAGTGA ATTATGAATC |
| | TTCACAGGTG AGCGTAGCTA GTTAAGCCTT TGTCTTTAAC CCTTGTCACT TAATACTTAG |
| 9301 | TTATACAGTT TTCTTTAACG TCACTAAATA GATGGACGCA AATAAATTTG TCGTTTACTT |
| | AATATGTCAA AAGAAATTGC AGTGATTTAT CTACCTGCGT TTATTTAAAC AGCAAATGAA |
| 9361 | AGTATAATGT ATGGAATGAG AATGTAGTTT GAATTGTTTT TTTTCTTTTC TTGCAGACTA |
| | TCATATTACA TACCTTACTC TTACATCAAA CTTAACAAAA AAAAGAAAAG AACGTCTGAT |
| | HindIII |
| | ˜˜˜˜˜˜ |
| | ClaI |
| | ˜˜˜˜˜˜˜ |
| 9421 | ATTCAAGAGG TGCGACGAAG AAGTTGCCGC GTTGGTAGTA GACGGTATCG ATAAGCTTGA |
| | TAAGTTCTCC ACGCTGCTTC TTCAACGGCG CAACCATCAT CTGCCATAGC TATTCGAACT |
| | PstI |
| | ˜˜˜˜˜˜ |
| | EcoRI |
| | ˜˜˜˜˜˜˜ |
| 9481 | TATCGAATTC CTGCAGCCCT GTAATACGAC TCACTATAGG GCGAATTGGG TACCGGGCCC |
| | ATAGCTTAAG GACGTCGGGA CATTATGCTG AGTGATATCC CGCTTAACCC ATGGCCCGGG |
| | HindIII PstI BamHI |
| | ˜˜˜˜˜˜˜ ˜˜˜˜˜˜ ˜˜˜˜˜˜ |
| | SmaI |
| | ˜˜˜˜˜˜˜ |
| | XmaI |
| | ˜˜˜˜˜˜˜ |
| | AvaI ClaI EcoRI AvaI NcoI |
| | ˜˜˜˜˜˜ ˜˜˜˜˜˜˜ ˜˜˜˜˜˜˜ ˜˜˜˜˜˜˜ ˜˜ |
| 9541 | CCCCTCGAGG TCGACGGTAT CGATAAGCTT GATATCGAAT TCCTGCAGCC CGGGGGATCC |
| | GGGGAGCTCC AGCTGCCATA GCTATTCGAA CTATAGCTTA AGGACGTCGG GCCCCCTAGG |
| | NcoI PstI PstI |
| | ˜˜˜˜ ˜˜˜˜˜˜˜ ˜˜ |
| | M A R S L L L P L Q I L L L S L A L E T |
| 9601 | ATGGCAAGAT CCCTTCTCCT GCCCCTGCAG ATCCTACTGC TATCCTTAGC CTTGGAAACT |
| | TACCGTTCTA GGGAAGAGGA CGGGGACGTC TAGGATGACG ATAGGAATCG GAACCTTTGA |
| | PstI |
| | ˜˜˜˜ |
| | A G E E A Q G D K I I D G A P C A R G S |
| 9661 | GCAGGAGAAG AAGCCCAGGG TGACAAGATT ATTGATGGCG CCCCATGTGC AAGAGGCTCC |
| | CGTCCTCTTC TTCGGGTCCC ACTGTTCTAA TAACTACCGC GGGGTACACG TTCTCCGAGG |
| | NcoI |
| | ˜˜˜˜˜˜ |
| | H P W Q V A L L S G N Q L H C G G V L V |
| 9721 | CACCCATGGC AGGTGGCCCT GCTCAGTGGC AATCAGCTCC ACTGCGGAGG CGTCCTGGTC |
| | GTGGGTACCG TCCACCGGGA CGAGTCACCG TTAGTCGAGG TGACGCCTCC GCAGGACCAG |
| | ApaLI |
| | ˜˜˜˜˜˜ |
| | N E R W V L T A A H C K M N E Y T V H L |
| 9781 | AATGAGCGCT GGGTGCTCAC TGCCGCCCAC TGCAAGATGA ATGAGTACAC CGTGCACCTG |
| | TTACTCGCGA CCCACGAGTG ACGGCGGGTG ACGTTCTACT TACTCATGTG GCACGTGGAC |
| | G S D T L G D R R A Q R I K A S K S F R |
| 9841 | GGCAGTGATA CGCTGGGCGA CAGGAGAGCT CAGAGGATCA AGGCCTCGAA GTCATTCCGC |
| | CCGTCACTAT GCGACCCGCT GTCCTCTCGA GTCTCCTAGT TCCGGAGCTT CAGTAAGGCG |
| | H P G Y S T Q T H V N D L M L V K L N S |
| 9901 | CACCCCGGCT ACTCCACACA GACCCATGTT AATGACCTCA TGCTCGTGAA GCTCAATAGC |
| | GTGGGGCCGA TGAGGTGTGT CTGGGTACAA TTACTGGAGT ACGAGCACTT CGAGTTATCG |
| | NcoI |
| | ˜˜˜˜˜˜˜ |
| | Q A R L S S M V K K V R L P S R C E P P |
| 9961 | CAGGCCAGGC TGTCATCCAT GGTGAAGAAA GTCAGGCTGC CCTCCCGCTG CGAACCCCCT |
| | GTCCGGTCCG ACAGTAGGTA CCACTTCTTT CAGTCCGACG GGAGGGCGAC GCTTGGGGGA |
| | G T T C T V S G W G T T T S P D V T F P |
| 10021 | GGAACCACCT GTACTGTCTC CGGCTGGGGC ACTACCACGA GCCCAGATGT GACCTTTCCC |
| | CCTTGGTGGA CATGACAGAG GCCGACCCCG TGATGGTGCT CGGGTCTACA CTGGAAAGGG |
| | S D L M C V D V K L I S P Q D C T K V Y |
| 10081 | TCTGACCTCA TGTGCGTGGA TGTCAAGCTC ATCTCCCCCC AGGACTGCAC GAAGGTTTAC |
| | AGACTGGAGT ACACGCACCT ACAGTTCGAG TAGAGGGGGG TCCTGACGTG CTTCCAAATG |
| | K D L L E N S M L C A G I P D S K K N A |
| 10141 | AAGGACTTAC TGGAAAATTC CATGCTGTGC GCTGGCATCC CCGACTCCAA GAAAAACGCC |
| | TTCCTGAATG ACCTTTTAAG GTACGACACG CGACCGTAGG GGCTGAGGTT CTTTTTGCGG |
| | C N G D S G G P L V C R G T L Q G L V S |
| 10201 | TGCAATGGTG ACTCAGGGGG ACCGTTGGTG TGCAGAGGTA CCCTGCAAGG TCTGGTGTCC |
| | ACGTTACCAC TGAGTCCCCC TGGCAACCAC ACGTCTCCAT GGGACGTTCC AGACCACAGG |
| | W G T F P C G Q P N D P G V Y T Q V C K |
| 10261 | TGGGGAACTT TCCCTTGCGG CCAACCCAAT GACCCAGGAG TCTACACTCA AGTGTGCAAG |
| | ACCCCTTGAA AGGGAACGCC GGTTGGGTTA CTGGGTCCTC AGATGTGAGT TCACACGTTC |
| | NotI |
| | ˜˜˜˜˜˜˜˜ |
| | F T K W I N D T M K K H R G G R G G G G |
| 10321 | TTCACCAAGT GGATAAATGA CACCATGAAA AAGCATCGC |
| | AAGTGGTTCA CCTATTTACT GTGGTACTTT TTCGTAGCG |
| | BstXI |
| | ˜˜˜˜˜˜˜˜˜˜˜˜˜ |
| | G G H H H H H H * |
| 10381 | CATC ATCATCATCA TCATTAACGC CACCGCGGTG GAGCTCCAGC TTTTGTTCCC |
| | GTAG TAGTAGTAGT AGTAATTGCG GTGGCGCCAC CTCGAGGTCG AAAACAAGGG |
| 10441 | TTTAGTGAGG GTTCGAGAAG TCTTACGAAC TTCCCGACGG TCAGGTCATC ACCATCGGAA |
| | AAATCACTCC CAAGCTCTTC AGAATGCTTG AAGGGCTGCC AGTCCAGTAG TGGTAGCCTT |
| 10501 | ACGAAAGATT CCGTTGCCCA GAGGCCCTCT TCCAACCCTC GTTCTTGGGT ATGGAAGCCA |
| | TGCTTTCTAA GGCAACGGGT CTCCGGGAGA AGGTTGGGAG CAAGAACCCA TACCTTCGGT |
| 10561 | ACGGAATCCA CGAAACCACA TACAACTCCA TCATGAAGTG CGACGTGGAC ATCCGTAAGG |
| | TGCCTTAGGT GCTTTGGTGT ATGTTGAGGT AGTACTTCAC GCTGCACCTG TAGGCATTCC |
| 10621 | ACTTGTACGC CAACACCGTA TTGTCCGGTG GTACCACCAT GTACCCTGGA ATCGCCGACC |
| | TGAACATGCG GTTGTGGCAT AACAGGCCAC CATGGTGGTA CATGGGACCT TAGCGGCTGG |
| 10681 | GTATGCAAAA GGAAATCACA CGTCTCGCCC CATCGACAAT GAAGATTAAG ATCATCGCTC |
| | CATACGTTTT CCTTTAGTGT GCAGAGCGGG GTAGCTGTTA CTTCTAATTC TAGTAGCGAG |
| | ClaI |
| | ˜˜˜˜˜˜˜ |
| 10741 | CCCCAGAGAG GAAGTACTCC GTATGGATCG GTGGATCGAT CCTCGCCTCC CTCTCTACCT |
| | GGGGTCTCTC CTTCATGAGG CATACCTAGC CACCTAGCTA GGAGCGGAGG GAGAGATGGA |
| 10801 | TCCAACAGAT GTGGATCTCG AAACAGGAGT ACGACGAGTC TGGTCCCTCC ATTGTACACA |
| | AGGTTGTCTA CACCTAGAGC TTTGTCCTCA TGCTGCTCAG ACCAGGGAGG TAACATGTGT |
| 10861 | GGAAGTGCTT CTAAGCGTTG AGACTTTAAG TTATGATGCC CTACAGCAGA ACCTCAAGAG |
| | CCTTCACGAA GATTCGCAAC TCTGAAATTC AATACTACGG GATGTCGTCT TGGAGTTCTC |
| 10921 | GGTGGCTCAA ATTACGCTTG TGATCTTGTA AATAAATTCA GTATTTAATG TAGGTTGTAA |
| | CCACCGAGTT TAATGCGAAC ACTAGAACAT TTATTTAAGT CATAAATTAC ATCCAACATT |
| 10981 | GGTATTGTAA TATGCATATT ACGTAAAACG AACGGAATGT TGTTGTTGCC GTTTTTTTTT |
| | CCATAACATT ATACGTATAA TGCATTTTGC TTGCCTTACA ACAACAACGG CAAAAAAAAA |
| 11041 | TGACAAAGAT TTTTATTTAT TAAAGTTACT AACCCCAAAA CTTTTTAATA AAATAAATTT |
| | ACTGTTTCTA AAAATAAATA ATTTCAATGA TTGGGGTTTT GAAAAATTAT TTTATTTAAA |
| 11101 | ATATACCGGT ATAATAACTG ACGTTTTTCA CTTGCTGTCC CCGCTCCCGA CTAACAGTAC |
| | TATATGGCCA TATTATTGAC TGCAAAAAGT GAACGACAGG GGCGAGGGCT GATTGTCATG |
| | ApaLI |
| | ˜˜˜˜˜˜˜ |
| 11161 | GTCGTGTGCA CCGAAATTAC CGATTTCGTA CACCGTTTGA GACAGTTACG CTAGGAGCAC |
| | CAGCACACGT GGCTTTAATG GCTAAAGCAT GTGGCAAACT CTGTCAATGC GATCCTCGTG |
| | PstI PstI |
| | ˜˜˜˜˜˜˜ ˜˜˜˜˜˜˜ |
| 11221 | AAATCTCCCA GCTGCATACC GTTGTTTACT GCAGCTCTGC AGTCTTTAAT TGGAATGCGA |
| | TTTAGAGGGT CGACGTATGG CAACAAATGA CGTCGAGACG TCAGAAATTA ACCTTACGCT |
| 11281 | GTCGTTGACC GCTTAATACG AAACATTCTA AAATTCGCAA AATGCAAAGG AAACTGGTTC |
| | CAGCAACTGG CGAATTATGC TTTGTAAGAT TTTAAGCGTT TTACGTTTCC TTTGACCAAG |
| 11341 | TGTACTTTCT ACCTTTCAAA AGATTCACCA AATTAATTTT ATGCGGACTC ACTAATTCCG |
| | ACATGAAAGA TGGAAAGTTT TCTAAGTGGT TTAATTAAAA TACGCCTGAG TGATTAAGGC |
| 11401 | TAGAAATCTG TGTAGAGGTA CCCAGGTTAC GCTTAGGCAT AAGATGACTG TTCGCGTTTT |
| | ATCTTTAGAC ACATCTCCAT GGGTCCAATG CGAATCCGTA TTCTACTGAC AAGCGCAAAA |
| 11461 | TACAATACAT ACGAGCAGGT TACACACAAG ATGAACATCC TTTGATGCGT CTGTGTCTTG |
| | ATGTTATGTA TGCTCGTCCA ATGTGTGTTC TACTTGTAGG AAACTACGCA GACACAGAAC |
| 11521 | ACCCGTCTGA GATTTGAGTG ACTTGTCAAC GTCATTGCGT AGTGTCACCG GTCGTCGAGA |
| | TGGGCAGACT CTAAACTCAC TGAACAGTTG CAGTAACGCA TCACAGTGGC CAGCAGCTCT |
| 11581 | TCCCCGCCGC GGTGGAGCTA CGAGCTC |
| | AGGGGCGGCG CCACCTCGAT GCTCGAG |

Fyn SCCE Preparation
”Gly_Fyn” into SCCE_Gly_His_pIE

Template:
Clone ID: IOH21081
Organism: Homo sapiens
Matching Nucleotide Accession: NM_{—}002037.3

Primers Used:

NotI Gly FYN F:
GGGGGGGGCGGCCGCGGGGGCGGGGGCGGGGGCGGGGGCGGGGGCACACT
CTTTGTGGCCCTTTATGAC

The highlight (Arial type face) shows the leading portion of the forward primer that lays down on the template.

Not Fyn R:
CCCCCCCGCGGCCGCCGTCAACTGGAGCCACATAATTGCTGGG

The highlight (Tahoma type face) shows the leading portion of the reverse primer that lays down on the template.
Method Sub-cloning using unique Restriction Sites
Preparation of Vector
SCCE_Gly_His6_pIE 10 μg (X μl)
10× NEB R.E. Buffer#3 10 μl
BSA 0.6 μl
R.E. NotI 3 μl
H₂O up to 60 μl
37° C. for 3 hours
Add 1 μl (1 U/μl) Alakaline phosphotase (Roche)
37° C. for 1 hour
Heat Inactivate the enzyme at 65° C. for 20 min
Purify digested vector by running on an agarose get and use QIAquick Gel Extraction Kit
Run aliquot of eluate (purified digested vector) for quantitation
Preparation of Insert:
1 μl of Not Gly FYN F (1 μg/μl)
1 μl of Not Fyn R (1 μg/μl)
250 ng (X μl) template
10 μl 10×TAQ Polymerase Buffer from NEB
0.8 μl dNTP (25 mM)
H₂O to a final volume of 100 μl
95° C. for 5 min
Ice; microfuge
Then add 1 μl of TAQ DNA Polymerase (NEB)
Step 1: 95° C. 30 seconds
Step 2: 95° C. 30 seconds

58° C. 1 minute

72° C. 1 minute/kb of pcr product length (1 min for FYN)

Repest step#2 29 times

Step 3: 72° C. for 10 min

Step 4: 4° C. pause

Check per by electrophoresis of 5 μl of the pcr product on a 1% agarose gel.

Purify the pcr product by using a QIAquick pcr purification Kit

Elute in 50 μl of elution buffer

Pcr product 50 μl

10×NEB R.E. Buffer#3 6 μl

BSA 0.6 μl

R.E. NotI 3 μl

H₂O up to 60 μl

37° C. overnight

Phenol Chloroform Extract

Purify digested pcr by running on an agarose get and use QIAquick Gel Extraction Kit

Run aliquot of eluate (purified digested insert) for quantitation

Ligation of Vector and Insert

Vector: SCCE-Gly_His6_pIE digested with NotI

Insert: Pcr product digested with NotI

| | | 10× | | |
|---|---|---|---|---|
| Vector | Insert | Ligation | | |
| (fmol) | (fmol) | Buffer (μl) | H₂O (μl) | Ligase (μl) |
| 30 | 60 | 2.5 | Upto 25 | 0.5 |
| 30 | 150 | 2.5 | Upto 25 | 0.5 |
| 30 | 0 | 2.5 | Upto 25 | 0.5 |

12° C. for 16 hours (overnight)
Transformation of Ligation Product into Competent C 7118 Cells
1. Gently thaw the competent C 7118 cells on ice. For each control and sample reaction to be transformed, aliquot 50 μl of the competent cells to a prechilled 15 ml conical tube.
2. Transfer 25 μl of each ligation product to separate aliquots of the competent cells. Swirl the transformation reactions gently to mix and incubate the reactions on ice for 30 minutes.
3. Heat pulse the transformation reactions for 3 min 37° C. and then place the reactions on ice for 2 minutes.
4. Add 0.8 ml of SOC medium and incubate the transformation reactions at 37° C. for 1.5 hours with shaking at 200-250 rpm.
5. Spin the tube at 3000 rpm for 5 min and re-suspend cells in 200 μl LB medium. Plate the cells on agar plates containing the ampicillin.
6. Incubate the transformation plates at 37° C. for >16 hours.
7. Next day, pick up a single colony, grow overnight in 3 ml LB medium.
8. Use QIAprep spin miniprep kit for plasmid purification.

9. The sequence was confirmed with the following sequencing primer:

| pIE Seq R: CGATGGTGATGACCTGACCGTC | |
|---|---|
| Sequence pIE Fyn SCCE | |
| 1 | CAGCTTTTGT TCCCTTTAGT GAGGGTTAAT TCCGAGCTTG GCGTAATCAT GGTCATAGCT |
| | GTCGAAAACA AGGGAAATCA CTCCCAATTA AGGCTCGAAC CGCATTAGTA CCAGTATCGA |
| 61 | GTTTCCTGTG TGAAATTGTT ATCCGCTCAC AATTCCACAC AACATACGAG CCGGAAGCAT |
| | CAAAGGACAC ACTTTAACAA TAGGCGAGTG TTAAGGTGTG TTGTATGCTC GGCCTTCGTA |
| 121 | AAAGTGTAAA GCCTGGGGTG CCTAATGAGT GAGCTAACTC ACATTAATTG CGTTGCGCTC |
| | TTTCACATTT CGGACCCCAC GGATTACTCA CTCGATTGAG TGTAATTAAC GCAACGCGAG |
| 181 | ACTGCCCGCT TTCCAGTCGG GAAACCTGTC GTGCCAGCTG CATTAATGAA TCGGCCAACG |
| | TGACGGGCGA AAGGTCAGCC CTTTGGACAG CACGGTCGAC GTAATTACTT AGCCGGTTGC |
| 241 | CGCGGGGAGA GGCGGTTTGC GTATTGGGCG CTCTTCCGCT TCCTCGCTCA CTGACTCGCT |
| | GCGCCCCTCT CCGCCAAACG CATAACCCGC GAGAAGGCGA AGGAGCGAGT GACTGAGCGA |
| 301 | GCGCTCGGTC GTTCGGCTGC GGCGAGCGGT ATCAGCTCAC TCAAAGGCGG TAATACGGTT |
| | CGCGAGCCAG CAAGCCGACG CCGCTCGCCA TAGTCGAGTG AGTTTCCGCC ATTATGCCAA |
| 361 | ATCCACAGAA TCAGGGGATA ACGCAGGAAA GAACATGTGA GCAAAAGGCC AGCAAAAGGC |
| | TAGGTGTCTT AGTCCCCTAT TGCGTCCTTT CTTGTACACT CGTTTTCCGG TCGTTTTCCG |
| 421 | CAGGAACCGT AAAAAGGCCG CGTTGCTGGC GTTTTTCCAT AGGCTCCGCC CCCCTGACGA |
| | GTCCTTGGCA TTTTTCCGGC GCAACGACCG CAAAAAGGTA TCCGAGGCGG GGGGACTGCT |
| 481 | GCATCACAAA AATCGACGCT CAAGTCAGAG GTGGCGAAAC CCGACAGGAC TATAAAGATA |
| | CGTAGTGTTT TTAGCTGCGA GTTCAGTCTC CACCGCTTTG GGCTGTCCTG ATATTTCTAT |
| 541 | CCAGGCGTTT CCCCCTGGAA GCTCCCTCGT GCGCTCTCCT GTTCCGACCC TGCCGCTTAC |
| | GGTCCGCAAA GGGGGACCTT CGAGGGAGCA CGCGAGAGGA CAAGGCTGGG ACGGCGAATG |
| 601 | CGGATACCTG TCCGCCTTTC TCCCTTCGGG AAGCGTGGCG CTTTCTCATA GCTCACGCTG |
| | GCCTATGGAC AGGCGGAAAG AGGGAAGCCC TTCGCACCGC GAAAGAGTAT CGAGTGCGAC |
| | ApaLI |
| | ˜˜˜˜˜˜˜ |
| 661 | TAGGTATCTC AGTTCGGTGT AGGTCGTTCG CTCCAAGCTG GGCTGTGTGC ACGAACCCCC |
| | ATCCATAGAG TCAAGCCACA TCCAGCAAGC GAGGTTCGAC CCGACACACG TGCTTGGGGG |
| 721 | CGTTCAGCCC GACCGCTGCG CCTTATCCGG TAACTATCGT CTTGAGTCCA ACCCGGTAAG |
| | GCAAGTCGGG CTGGCGACGC GGAATAGGCC ATTGATAGCA GAACTCAGGT TGGGCCATTC |
| 781 | ACACGACTTA TCGCCACTGG CAGCAGCCAC TGGTAACAGG ATTAGCAGAG CGAGGTATGT |
| | TGTGCTGAAT AGCGGTGACC GTCGTCGGTG ACCATTGTCC TAATCGTCTC GCTCCATACA |
| 841 | AGGCGGTGCT ACAGAGTTCT TGAAGTGGTG GCCTAACTAC GGCTACACTA GAAGGACAGT |
| | TCCGCCACGA TGTCTCAAGA ACTTCACCAC CGGATTGATG CCGATGTGAT CTTCCTGTCA |
| 901 | ATTTGGTATC TGCGCTCTGC TGAAGCCAGT TACCTTCGGA AAAAGAGTTG GTAGCTCTTG |
| | TAAACCATAG ACGCGAGACG ACTTCGGTCA ATGGAAGCCT TTTTCTCAAC CATCGAGAAC |
| 961 | ATCCGGCAAA CAAACCACCG CTGGTAGCGG TGGTTTTTTT GTTTGCAAGC AGCAGATTAC |
| | TAGGCCGTTT GTTTGGTGGC GACCATCGCC ACCAAAAAAA CAAACGTTCG TCGTCTAATG |
| 1021 | GCGCAGAAAA AAAGGATCTC AAGAAGATCC TTTGATCTTT TCTACGGGGT CTGACGCTCA |
| | CGCGTCTTTT TTTCCTAGAG TTCTTCTAGG AAACTAGAAA AGATGCCCCA GACTGCGAGT |
| 1081 | GTGGAACGAA AACTCACGTT AAGGGATTTT GGTCATGAGA TTATCAAAAA GGATCTTCAC |
| | CACCTTGCTT TTGAGTGCAA TTCCCTAAAA CCAGTACTCT AATAGTTTTT CCTAGAAGTG |
| 1141 | CTAGATCCTT TTAAATTAAA AATGAAGTTT TAAATCAATC TAAAGTATAT ATGAGTAAAC |
| | GATCTAGGAA AATTTAATTT TTACTTCAAA ATTTAGTTAG ATTTCATATA TACTCATTTG |
| 1201 | TTGGTCTGAC AGTTACCAAT GCTTAATCAG TGAGGCACCT ATCTCAGCGA TCTGTCTATT |
| | AACCAGACTG TCAATGGTTA CGAATTAGTC ACTCCGTGGA TAGAGTCGCT AGACAGATAA |
| 1261 | TCGTTCATCC ATAGTTGCCT GACTCCCCGT CGTGTAGATA ACTACGATAC GGGAGGGCTT |
| | AGCAAGTAGG TATCAACGGA CTGAGGGGCA GCACATCTAT TGATGCTATG CCCTCCCGAA |
| 1321 | ACCATCTGGC CCCAGTGCTG CAATGATACC GCGAGACCCA CGCTCACCGG CTCCAGATTT |
| | TGGTAGACCG GGGTCACGAC GTTACTATGG CGCTCTGGGT GCGAGTGGCC GAGGTCTAAA |
| 1381 | ATCAGCAATA AACCAGCCAG CCGGAAGGGC CGAGCGCAGA AGTGGTCCTG CAACTTTATC |
| | TAGTCGTTAT TTGGTCGGTC GGCCTTCCCG GCTCGCGTCT TCACCAGGAC GTTGAAATAG |
| 1441 | CGCCTCCATC CAGTCTATTA ATTGTTGCCG GGAAGCTAGA GTAAGTAGTT CGCCAGTTAA |
| | GCGGAGGTAG GTCAGATAAT TAACAACGGC CCTTCGATCT CATTCATCAA GCGGTCAATT |
| 1501 | TAGTTTGCGC AACGTTGTTG CCATTGCTAC AGGCATCGTG GTGTCACGCT CGTCGTTTGG |
| | ATCAAACGCG TTGCAACAAC GGTAACGATG TCCGTAGCAC CACAGTGCGA GCAGCAAACC |
| 1561 | TATGGCTTCA TTCAGCTCCG GTTCCCAACG ATCAAGGCGA GTTACATGAT CCCCCATGTT |
| | ATACCGAAGT AAGTCGAGGC CAAGGGTTGC TAGTTCCGCT CAATGTACTA GGGGGTACAA |
| 1621 | GTGCAAAAAA GCGGTTAGCT CCTTCGGTCC TCCGATCGTT GTCAGAAGTA AGTTGGCCGC |
| | CACGTTTTTT CGCCAATCGA GGAAGCCAGG AGGCTAGCAA CAGTCTTCAT TCAACCGGCG |
| 1681 | AGTGTTATCA CTCATGGTTA TGGCAGCACT GCATAATTCT CTTACTGTCA TGCCATCCGT |
| | TCACAATAGT GAGTACCAAT ACCGTCGTGA CGTATTAAGA GAATGACAGT ACGGTAGGCA |
| 1741 | AAGATGCTTT TCTGTGACTG GTGAGTACTC AACCAAGTCA TTCTGAGAAT AGTGTATGCG |
| | TTCTACGAAA AGACACTGAC CACTCATGAG TTGGTTCAGT AAGACTCTTA TCACATACGC |
| 1801 | GCGACCGAGT TGCTCTTGCC CGGCGTCAAT ACGGGATAAT ACCGCGCCAC ATAGCAGAAC |
| | CGCTGGCTCA ACGAGAACGG GCCGCAGTTA TGCCCTATTA TGGCGCGGTG TATCGTCTTG |
| 1861 | TTTAAAAGTG CTCATCATTG GAAAACGTTC TTCGGGGCGA AAACTCTCAA GGATCTTACC |
| | AAATTTTCAC GAGTAGTAAC CTTTTGCAAG AAGCCCCGCT TTTGAGAGTT CCTAGAATGG |
| | ApaLI |
| | ˜˜˜˜˜˜ |
| 1921 | GCTGTTGAGA TCCAGTTCGA TGTAACCCAC TCGTGCACCC AACTGATCTT CAGCATCTTT |
| | CGACAACTCT AGGTCAAGCT ACATTGGGTG AGCACGTGGG TTGACTAGAA GTCGTAGAAA |
| 1981 | TACTTTCACC AGCGTTTCTG GGTGAGCAAA AACAGGAAGG CAAAATGCCG CAAAAAAGGG |
| | ATGAAAGTGG TCGCAAAGAC CCACTCGTTT TTGTCCTTCC GTTTTACGGC GTTTTTTCCC |
| 2041 | AATAAGGGCG ACACGGAAAT GTTGAATACT CATACTCTTC CTTTTTCAAT ATTATTGAAG |
| | TTATTCCCGC TGTGCCTTTA CAACTTATGA GTATGAGAAG GAAAAAGTTA TAATAACTTC |
| 2101 | CATTTATCAG GGTTATTGTC TCATGAGCGG ATACATATTT GAATGTATTT AGAAAAATAA |
| | GTAAATAGTC CCAATAACAG AGTACTCGCC TATGTATAAA CTTACATAAA TCTTTTTATT |
| 2161 | ACAAATAGGG GTTCCGCGCA CATTTCCCCG AAAAGTGCCA CCTGGGAAAT TGTAAACGTT |
| | TGTTTATCCC CAAGGCGCGT GTAAAGGGGC TTTTCACGGT GGACCCTTTA ACATTTGCAA |
| 2221 | AATATTTTGT TAAAATTCGC GTTAAATTTT TGTTAAATCA GCTCATTTTT TAACCAATAG |
| | TTATAAAACA ATTTTAAGCG CAATTTAAAA ACAATTTAGT CGAGTAAAAA ATTGGTTATC |
| 2281 | GCCGAAATCG GCAAAATCCC TTATAAATCA AAAGAATAGA CCGAGATAGG GTTGAGTGTT |
| | CGGCTTTAGC CGTTTTAGGG AATATTTAGT TTTCTTATCT GGCTCTATCC CAACTCACAA |
| 2341 | GTTCCAGTTT GGAACAAGAG TCCACTATTA AAGAACGTGG ACTCCAACGT CAAAGGGCGA |
| | CAAGGTCAAA CCTTGTTCTC AGGTGATAAT TTCTTGCACC TGAGGTTGCA GTTTCCCGCT |
| 2401 | AAAACCGTCT ATCAGGGCGA TGGCCCACTA CGTGAACCAT CACCCTAATC AAGTTTTTTG |
| | TTTTGGCAGA TAGTCCCGCT ACCGGGTGAT GCACTTGGTA GTGGGATTAG TTCAAAAAAC |
| 2461 | GGGTCGAGGT GCCGTAAAGC ACTAAATCGG AACCCTAAAG GGAGCCCCCG ATTTAGAGCT |
| | CCCAGCTCCA CGGCATTTCG TGATTTAGCC TTGGGATTTC CCTCGGGGGC TAAATCTCGA |
| 2521 | TGACGGGGAA AGCCGGCGAA CGTGGCGAGA AAGGAAGGGA AGAAAGCGAA AGGAGCGGGC |
| | ACTGCCCCTT TCGGCCGCTT GCACCGCTCT TTCCTTCCCT TCTTTCGCTT TCCTCGCCCG |
| 2581 | GCTAGGGCGC TGGCAAGTGT AGCGGTCACG CTGCGCGTAA CCACCACACC CGCCGCGCTT |
| | CGATCCCGCG ACCGTTCACA TCGCCAGTGC GACGCGCATT GGTGGTGTGG GCGGCGCGAA |
| 2641 | AATGCGCCGC TACAGGGCGC GTCGCGCCAT TCGCCATTCA GGCTGCGCAA CTGTTGGGAA |
| | TTACGCGGCG ATGTCCCGCG CAGCGCGGTA AGCGGTAAGT CCGACGCGTT GACAACCCTT |
| 2701 | GGGCGATCGG TGCGGGCCTC TTCGCTATTA CGCCAGCTGG CGAAAGGGGG ATGTGCTGCA |
| | CCCGCTAGCC ACGCCCGGAG AAGCGATAAT GCGGTCGACC GCTTTCCCCC TACACGACGT |
| 2761 | AGGCGATTAA GTTGGGTAAC GCCAGGGTTT TCCCAGTCAC GACGTTGTAA AACGACGGCC |
| | TCCGCTAATT CAACCCATTG CGGTCCCAAA AGGGTCAGTG CTGCAACATT TTGCTGCCGG |
| | ClaI |
| | ˜˜˜˜˜ |
| 2821 | AGTGAATTGT AATACGACTC ACTATAGGGC GAATTGGGTA CCGGGCCTCG ACGGTATCGA |
| | TCACTTAACA TTATGCTGAG TGATATCCCG CTTAACCCAT GGCCCGGAGC TGCCATAGCT |
| | ClaI |
| | ˜ |
| 2881 | TTGCAGGTCG ATATTTAAAA AAAATTATAA TAATGTTAAA GTTGCTTCAT ACGTTGAAGT |
| | AACGTCCAGC TATAAATTTT TTTTAATATT ATTACAATTT CAACGAAGTA TGCAACTTCA |
| 2941 | ACCTTACACA ACAAATAATG CAGACGTCGA AATGACTGAC ATAACAAATG CGCCCTTTCG |
| | TGGAATGTGT TGTTTATTAC GTCTGCAGCT TTACTGACTG TATTGTTTAC GCGGGAAAGC |
| 3001 | CCCAAAATCT AAAGCAAGGA GAAGATTAGA TTTTACCAAC ATGGCGCCGC AGCCGTGTTG |
| | GGGTTTTAGA TTTCGTTCCT CTTCTAATCT AAAATGGTTG TACCGCGGCG TCGGCACAAC |
| 3061 | TATTGACGAC GGCAATTTTG CAAAACTTTA CTGTGATATA TTTATTAAAT TAAGTTTGCT |
| | ATAACTGCTG CCGTTAAAAC GTTTTGAAAT GACACTATAT AAATAATTTA ATTCAAACGA |
| 3121 | TTAAAATGAG TTTTTTTACA AATCTTCGCA GAGTCAATAA ATTGTATCCT AATCAGGCCA |
| | AATTTTACTC AAAAAAATGT TTAGAAGCGT CTCAGTTATT TAACATAGGA TTAGTCCGGT |
| 3181 | GTTTTCTTGC TGATAATACG CGTCTTTTAA CAAGGCACTC CCGCCGGTTT CACAAATGTG |
| | CAAAAGAACG ACTATTATGC GCAGAAAATT GTTCCGTGAG GGCGGCCAAA GTGTTTACAC |
| 3241 | CTCAACGCGC CCAGTGTACG CAACCTTGGA AACAACAGAT ATGGGCCGGG CTATCAATTA |
| | GAGTTGCGCG GGTCACATGC GTTGGAACCT TTGTTGTCTA TACCCGGCCC GATAGTTAAT |
| 3301 | TCTAACAACC GGTTTGTGAG CACTTCAGAC ATAAACAGAA TTACTCGTAA CAACGATGTC |
| | AGATTGTTGG CCAAACACTC GTGAAGTCTG TATTTGTCTT AATGAGCATT GTTGCTACAG |
| 3361 | CCCAACATAC GCGGAGTATT TCAGGGCATT TCAGACCCTC AAATAAACTC ATTGAGCCAA |
| | GGGTTGTATG CGCCTCATAA AGTCCCGTAA AGTCTGGGAG TTTATTTGAG TAACTCGGTT |
| 3421 | TTGCGGCGCA TGGACCAACG TGCCAGACTT TCATTACCAC ACCAAACAGA CGCGATCCAA |
| | AACGCCGCGT ACCTGGTTGC ACGGTCTGAA AGTAATGGTG TGGTTTGTCT GCGCTAGGTT |
| 3481 | TGCAGTCAGA CAAAACTTCC CGGAGACCAA CGTGCGCACG CCCGAAGGTG TTCAAAATGC |
| | ACGTCAGTCT GTTTTGAAGG GCCTCTGGTT GCACGCGTGC GGGCTTCCAC AAGTTTTACG |
| | PstI |
| | ˜˜˜˜˜˜˜ |
| 3541 | ACTGCAGCAA AACCCCCCCG TTTACATAAT CACATGAGAA CCTTGAAAGT AGCAGGAGTG |
| | TGACGTCGTT TTGGGGGGGC AAATGTATTA GTGTACTCTT GGAACTTTCA TCGTCCTCAC |
| 3601 | GGCATACTCT TGGGCCGGCG GCGGTTATCT TTTGTTTACC GCCGCCACAT TAGTACAAGA |
| | CCGTATGAGA ACCCGGCCGC CGCCAATAGA AAACAAATGG CGGCGGTGTA ATCATGTTCT |
| 3661 | TATAATCAAC GCCATCAATA GAACCGGCGG AAGTTATTAT GTGCAAGGTA GAAACGCCGG |
| | ATATTAGTTG CGGTAGTTAT CTTGGCCGCC TTCAATAATA CACGTTCCAT CTTTGCGGCC |
| 3721 | AGAAAACGCC GAGGCCTGTT TGTTATTGCA GCGCACTTGT CGTCAAGACC GCAATCTAGC |
| | TCTTTTGCGG CTCCGGACAA ACAATAACGT CGCGTGAACA GCAGTTCTGG CGTTAGATCG |
| 3781 | TCAGTCGGAT GTTAACATTT GCTCAAGAGA CCCCTTGTTG GCTAACGATT CGCCCCTACT |
| | AGTCAGCCTA CAATTGTAAA CGAGTTCTCT GGGGAACAAC CGATTGCTAA GCGGGGATGA |
| 3841 | AACCAACATG TGCCAAGGAT TTAACTATGA AACAGAAAAA ACAGTTTGTC GCGGCAGCAA |
| | TTGGTTGTAC ACGGTTCCTA AATTGATACT TTGTCTTTTT TGTCAAACAG CGCCGTCGTT |
| 3901 | TCCGGCCGCT AACCCAACTT CGCCTCAATA CGTAGATATT AGCGATCTTC TGCGGGCCAA |
| | AGGCCGGCGA TTGGGTTGAA GCGGAGTTAT GCATCTATAA TCGCTAGAAG ACGCCCGGTT |
| 3961 | ACAATCATGT GCATCGAACC TTACACGTTT AGTGATTTAA TTGGCGACTT GCGTTTACAT |
| | TGTTAGTACA CGTAGCTTGG AATGTGCAAA TCACTAAATT AACCGCTGAA CGCAAATGTA |
| 4021 | TGGTTACTGG GAAGAGAAGG TTTAATCGGC AAATCGTCCA ACGGTAGTGA CAGCATCCGC |
| | ACCAATGACC CTTCTCTTCC AAATTAGCCG TTTAGCAGGT TGCCATCACT GTCGTAGGCG |
| 4081 | AACAAAATAA TGCCTCATCA TTATGATGAT AGGCGCGTTC TTGTTTTTAG GTTTAATACT |
| | TTGTTTTATT ACGGAGTAGT AATACTACTA TCCGCGCAAG AACAAAAATC CAAATTATGA |
| 4141 | TTATTTTATC TACAGATACA TGACAAAAGG AGGAGGAGGA GGAGGAAGCG GTGGGGCACC |
| | AATAAAATAG ATGTCTATGT ACTGTTTTCC TCCTCCTCCT CCTCCTTCGC CACCCCGTGG |
| 4201 | AACTCCCATT GTTGTTATTA TGCAACACCC CACATCAACA GCGGCCCCTC GTCGATAATA |
| | TTGAGGGTAA CAACAATAAT ACGTTGTGGG GTGTAGTTGT CGCCGGGGAG CAGCTATTAT |
| 4261 | AAAGACAAAA ATAATATAAA ATATATGTAT AATTAATTAA ATTCAAAAGA TATGTATAAT |
| | TTTCTGTTTT TATTATATTT TATATACATA TTAATTAATT TAAGTTTTCT ATACATATTA |
| 4321 | TAATTAAATT CAAATTTTTT ATATTTACAA TTTAGTTTTT GTTCCGCAAA CGTTATAGCG |
| | ATTAATTTAA GTTTAAAAAA TATAAATGTT AAATCAAAAA CAAGGCGTTT GCAATATCGC |
| 4381 | TCGGACAACG GAACCAGACC CTGTAATATT AAAGCTAACA ATTTTAACAA ATTATTGTGC |
| | AGCCTGTTGC CTTGGTCTGG GACATTATAA TTTCGATTGT TAAAATTGTT TAATAACACG |
| 4441 | AATGTAGTGC TCTCTCTTCG GTTCACTTTA CTGATTACAA ACATGTGATG CTTAAATCTA |
| | TTACATCACG AGAGAGAAGC CAAGTGAAAT GACTAATGTT TGTACACTAC GAATTTAGAT |
| 4501 | TTATATTTTT GAATTACTTG ACTAGCGTCT ACATCTTTAA TCTCGCCAGA AATCCAATAA |
| | AATATAAAAA CTTAATGAAC TGATCGCAGA TGTAGAAATT AGAGCGGTCT TTAGGTTATT |
| 4561 | AACTCTTCGT TTTTCTTAGC TATAGTCAAC CGCTCTTCGT TTTTGAAAGA CAATACTATA |
| | TTGAGAAGCA AAAAGAATCG ATATCAGTTG GCGAGAAGCA AAAACTTTCT GTTATGATAT |
| 4621 | AAATTGTGAC CTTTTACATT ATCCACATTC TGAGTCAAAT ACTGTTCGAC AATGTGCATG |
| | TTTAACACTG GAAAATGTAA TAGGTGTAAG ACTCAGTTTA TGACAAGCTG TTACACGTAC |
| 4681 | CTGCCGTCCT CCTTCTTAAC CTTTTTTAAA TTTTCAGCGT TATTATTACT CGCAATATTG |
| | GACGGCAGGA GGAAGAATTG GAAAAAATTT AAAAGTCGCA ATAATAATGA GCGTTATAAC |
| 4741 | TCATGATATT TATAATTATT AAACAAAAGA TTAGCGACAC TACTGTATTT GTACGTGAGC |
| | AGTACTATAA ATATTAATAA TTTGTTTTCT AATCGCTGTG ATGACATAAA CATGCACTCG |
| 4801 | GTACTTTTTT TGTTAACAAT TAAATTTAAA TTGTCCACCA CATATTTGTT TGGGGGATTG |
| | CATGAAAAAA ACAATTGTTA ATTTAAATTT AACAGGTGGT GTATAAACAA ACCCCCTAAC |
| 4861 | TCGGGAAACT TTACACTTTC CGAATACTTT AATATTTGAC TCACATACGG CGATACAAAA |
| | AGCCCTTTGA AATGTGAAAG GCTTATGAAA TTATAAACTG AGTGTATGCC GCTATGTTTT |
| 4921 | AAATTATTAG ATGCAGTCTC AATTTCATTA CTCTCTTTAC GACTAAGCAT AATAGGCAAA |
| | TTTAATAATC TACGTCAGAG TTAAAGTAAT GAGAGAAATG CTGATTCGTA TTATCCGTTT |
| 4981 | GTAAATAAAT TTTTATCTTG ATACATTTCG TACAACTTGC TCAAAAGAAA CCCACACTTT |
| | CATTTATTTA AAAATAGAAC TATGTAAAGC ATGTTGAACG AGTTTTCTTT GGGTGTGAAA |
| 5041 | CTTTCGCCCA ACGATTGTAA CAAAGTCACA AATGTGGTTT GCGCGTAATA CATATCTAAA |
| | GAAAGCGGGT TGCTAACATT GTTTCAGTGT TTACACCAAA CGCGCATTAT GTATAGATTT |
| 5101 | TTAAAATATG AAGTCAGAGC AGCTTTAAAC GTGTGATGCA CATCGACAAA GTGGCATTTT |
| | AATTTTATAC TTCAGTCTCG TCGAAATTTG CACACTACGT GTAGCTGTTT CACCGTAAAA |
| 5161 | TTACAATTTT GTGCAGCCGT CTCGTCGTTG CACACATCTT GAGAATGAGG AATTTCTATG |
| | AATGTTAAAA CACGTCGGCA GAGCAGCAAC GTGTGTAGAA CTCTTACTCC TTAAAGATAC |
| 5221 | CCGGTTTCTT TAACCAAATT GTACGAGATC ATAAATCTAA TTTTATCAAA AGTTACCACA |
| | GGCCAAAGAA ATTGGTTTAA CATGCTCTAG TATTTAGATT AAAATAGTTT TCAATGGTGT |
| 5281 | AACACGCGAT TATCTACCAT GTAATAGTTG TTTGTATATT CGTACACCAC ATTGCTCACG |
| | TTGTGCGCTA ATAGATGGTA CATTATCAAC AAACATATAA GCATGTGGTG TAACGAGTGC |
| 5341 | TACTTGGCAA ATATAATTTC AAACGGCTTT ACTTCACTTT TTTTAACCAC AAACATGTAA |
| | ATGAACCGTT TATATTAAAG TTTGCCGAAA TGAAGTGAAA AAAATTGGTG TTTGTACATT |
| 5401 | TAACCAGTTT CGGACATATG GTCGGAGAAC CTATTGGAAT TGTAGTCGTT GTCGTCGAAA |
| | ATTGGTCAAA GCCTGTATAC CAGCCTCTTG GATAACCTTA ACATCAGCAA CAGCAGCTTT |
| 5461 | CGCATCAAAT ACGGCGCAAA ATCATTAGTA AAATAATGCG TAATTTCTTG AGTTGAAGCA |
| | GCGTAGTTTA TGCCGCGTTT TAGTAATCAT TTTATTACGC ATTAAAGAAC TCAACTTCGT |
| 5521 | ACCGTGCAAA TGTTCGTGTT GTGATTAATT GTCTGCTCAA GGGTTGCACA GCTTTGAATT |
| | TGGCACGTTT ACAAGCACAA CACTAATTAA CAGACGAGTT CCCAACGTGT CGAAACTTAA |
| 5581 | GTGCTTTTCT TGTATTTAGG CTTCAATTTA TTCTTGTTAA ATTGGCCCAC CACACTTTGT |
| | CACGAAAAGA ACATAAATCC GAAGTTAAAT AAGAACAATT TAACCGGGTG GTGTGAAACA |
| 5641 | GAATCGTCCA AGTATTCGTC CAGCTTCCGT TTAGTTCCAG TTGCCGATGG TTGGTTCACA |
| | CTTAGCAGGT TCATAAGCAG GTCGAAGGCA AATCAAGGTC AACGGCTACC AACCAAGTGT |
| 5701 | CCAACAGGAT GCTCAAAAGA TTCCGCATTA TAAGCAGAAC TGGGCGATGG TTGCTCCGCA |
| | GGTTGTCCTA CGAGTTTTCT AAGGCGTAAT ATTCGTCTTG ACCCGCTACC AACGAGGCGT |
| 5761 | ACAGGCAGCT CAAAAGATTC CGCATTATAA GCAGAACTAA CTGCTTCTCC GAGATTATCA |
| | TGTCCGTCGA GTTTTCTAAG GCGTAATATT CGTCTTGATT GACGAAGAGG CTCTAATAGT |
| 5821 | GTGGTCTTGA GCAAACATTC CATTATATCG TTATCATCAG TTAACGAATT GACGCTTGCC |
| | CACCAGAACT CGTTTGTAAG GTAATATAGC AATAGTAGTC AATTGCTTAA CTGCGAACGG |
| | PstI |
| | ˜˜˜˜˜˜˜ |
| 5881 | AAAAAGTTTG AAGCTGCCTG CAGTCTGCTG TCAGATACTA CCGTGTCGGC TCCATCCGGC |
| | TTTTTCAAAC TTCGACGGAC GTCAGACGAC AGTCTATGAT GGCACAGCCG AGGTAGGCCG |
| 5941 | GTGGGATTGT TATAATAATT CAAATAGTCG TTGGGCTGTT GTTTATCACA AAACTCTGAA |
| | CACCCTAACA ATATTATTAA GTTTATCAGC AACCCGACAA CAAATAGTGT TTTGAGACTT |
| | AvaI |
| | ˜˜˜˜˜˜˜ |
| 6001 | TAGCCGTTGT CGAACGACGC TCGGGACGGC GTCGGAGCAC TGGTGTACGA CGCGTTAAAA |
| | ATCGGCAACA GCTTGCTGCG AGCCCTGCCG CAGCCTCGTG ACCACATGCT GCGCAATTTT |
| 6061 | TTAATTTGCG TCATAGTCGT TTGGTTGTTC ACGATCGTGT CCCGCCAATG TCAACTTGCA |
| | AATTAAACGC AGTATCAGCA AACCAACAAG TGCTAGCACA GGGCGGTTAC AGTTGAACGT |
| 6121 | ACTGAAACAA TATTCAACAT GAACGTCAAT TTATACTGCC CTAATGGCGA ACACGATAAT |
| | TGACTTTGTT ATAAGTTGTA CTTGCAGTTA AATATGACGG GATTACCGCT TGTGCTATTA |
| 6181 | AATATTTTTT TTATTATGCC CTCTAAAACC AATGCGGTTA TCGTTTATTT ATTCAAATTA |
| | TTATAAAAAA AATAATACGG GAGATTTTGG TTACGCCAAT AGCAAATAAA TAAGTTTAAT |
| 6241 | GATACAGAAC ATCCGCCGAC ATACAATGTT AATGCAAAAA CTCGTTTGGT GAGCGGATAC |
| | CTATGTCTTG TAGGCGGCTG TATGTTACAA TTACGTTTTT GAGCAAACCA CTCGCCTATG |
| 6301 | GAAAACAGTC GGCCGATAAA CATTAATCTG AGGTCGATAA CACCGTCCTT GAACGGAACA |
| | CTTTTGTCAG CCGGCTATTT GTAATTAGAC TCCAGCTATT GTGGCAGGAA CTTGCCTTGT |
| 6361 | CGAGGAGCGT ACGTGATCAG CTGCATTCGC GCGCCGCGCC TTTATCGAGA TTTATTTACA |
| | GCTCCTCGCA TGCACTAGTC GACGTAAGCG CGCGGCGCGG AAATAGCTCT AAATAAATGT |
| 6421 | TACAACAAGT ACACTGCGCC GTTGGCATTT GTGGTAACGC GCACACAAGC AGAGCTGCAA |
| | ATGTTGTTCA TGTGACGCGG CAACCGTAAA CACCATTGCG CGTGTGTTCG TCTCGACGTT |
| 6481 | GTGTGGCACA TTTTGTCTGT GCGCAAAACC TTTGAAGCCA AAAGCACAAG GTCCGTTACG |
| | CACACCGTGT AAAACAGACA CGCGTTTTGG AAACTTCGGT TTTCGTGTTC CAGGCAATGC |
| 6541 | GGCATGCTAG CGCACACGGA CAACGGACCC GACAAATTCT ACGCCAAGGA TTTAATGATA |
| | CCGTACGATC GCGTGTGCCT GTTGCCTGGG CTGTTTAAGA TGCGGTTCCT AAATTACTAT |
| 6601 | ATGTCGGGCA ACGTGTCGGT GCATTTTATT AATAACTTAC AAAATGTCGC GCGCATCACA |
| | TACAGCCCGT TGCACAGCCA CGTAAAATAA TTATTGAATG TTTTACAGCG CGCGTAGTGT |
| | HindIII EcoRI |
| | ˜˜˜˜˜˜˜ ˜˜˜ |
| 6661 | AAGACATTGA TATATTTAAA CATTTATGTC CCGAACTGCA ACGATAAGCT TGATATCGAA |
| | TTCTGTAACT ATATAAATTT GTAAATACAG GGCTTGACGT TGCTATTCGA ACTATAGCTT |
| | PstI |
| | ˜˜˜˜˜˜ |
| | EcoRI |
| | ˜˜˜ |
| 6721 | TTCCTGCAGC CCAATATTAC GTTCGTGCCA GAAATTAATT TCTCCGCGTC GTATTATACG |
| | AAGGACGTCG GGTTATAATG CAAGCACGGT CTTTAATTAA AGAGGCGCAG CATAATATGC |
| 6781 | ATTTATACGG TACAGCAGCT TGGCCCACAA ATAGATCGTT TTATGATTTT GATGATGGAG |
| | TAAATATGCC ATGTCGTCGA ACCGGGTGTT TATCTAGCAA AATACTAAAA CTACTACCTC |
| 6841 | GTGCGCTCAA GATGAAACCC ATTCAGACGT TATTAGTTGC GTCAAGTATT TGGCAATTTG |
| | CACGCGAGTT CTACTTTGGG TAAGTCTGCA ATAATCAACG CAGTTCATAA ACCGTTAAAC |
| 6901 | CTACGACGCA ATTATTGTGG AAGAAGCGTA ATTTGTGAAC AGCCCATTCG AGGCTAGATT |
| | GATGCTGCGT TAATAACACC TTCTTCGCAT TAAACACTTG TCGGGTAAGC TCCGATCTAA |
| 6961 | GAAAAAGTAT ATTGATATTA AATCATATAA ATTGTTTATG AGGCCTTCAA ACGAATCTTG |
| | CTTTTTCATA TAACTATAAT TTAGTATATT TAACAAATAC TCCGGAAGTT TGCTTAGAAC |
| 7021 | TAAAGATTAT TTATTAAAAT TGTTCAACGA TTGTATGAGA GGGTCATTTG TTTTTCAAAA |
| | ATTTCTAATA AATAATTTTA ACAAGTTGCT AACATACTCT CCCAGTAAAC AAAAAGTTTT |
| | EcoRI |
| | ˜˜˜˜˜˜ |
| 7081 | CTGAACTCGC TTTACGAGTA GAATTCTACT TGTAAAACAC AATCAAGAGA TGATGTCATT |
| | GACTTGAGCG AAATGCTCAT CTTAAGATGA ACATTTTGTG TTAGTTCTCT ACTACAGTAA |
| 7141 | TGTTTTTCAA AACTGAATGA TGTCATTTGT TTTTTAAAAC TAAACTCGCT TTTACGAGTA |
| | ACAAAAAGTT TTGACTTACT ACAGTAAACA AAAAATTTTG ATTTGAGCGA AAATGCTCAT |
| | EcoRI |
| | ˜˜˜˜˜˜ |
| 7201 | GAATTCTACG TGTAAAACAT AATCAAGAGA TGATGTCATT TGTTTTTCAA AACTGAACCG |
| | CTTAAGATGC ACATTTTGTA TTAGTTCTCT ACTACAGTAA ACAAAAAGTT TTGACTTGGC |
| | EcoRI |
| | ˜˜˜˜˜˜ |
| 7261 | GCTTTACGAG TAGAATTCTA CGTGTAAAAC ATAATCAAGA GATGATGTCA TCATTAAACT |
| | CGAAATGCTC ATCTTAAGAT GCACATTTTG TATTAGTTCT CTACTACAGT AGTAATTTGA |
| 7321 | GATGTCATTT TTATACACGA TTGTTAACAT GTTTAATAAT GACTAATTTG TTTTTCAAAT |
| | CTACAGTAAA AATATGTGCT AACAATTGTA CAAATTATTA CTGATTAAAC AAAAAGTTTA |
| | EcoRI |
| | ˜˜˜˜˜˜˜ |
| 7381 | TAAACTCGCT TTACAAGTAG AATTCTACTT GTAACGCACG ATTAAAATTA TTATAATCAG |
| | ATTTGAGCGA AATGTTCATC TTAAGATGAA CATTGCGTGC TAATTTTAAT AATATTAGTC |
| 7441 | GAATGATGTC ATTTGTTTTC GTCATAAAAT GTTTATACAA CGGAATCTTC TTGTAAATTA |
| | CTTACTACAG TAAACAAAAG CAGTATTTTA CAAATATGTT GCCTTAGAAG AACATTTAAT |
| 7501 | TCCAAATAAT ATAATTTATC CGATTCTACG TTACATTTAA ATTCGTTGTT ATCGTACAAT |
| | AGGTTTATTA TATTAAATAG GCTAAGATGC AATGTAAATT TAAGCAACAA TAGCATGTTA |
| 7561 | TCTTCAGGAC ACGCCATGTA TTGGCCGTTT TTAACGTGCA ACCAACGATT GTATTTGACG |
| | AGAAGTCCTG TGCGGTACAT AACCGGCAAA AATTGCACGT TGGTTGCTAA CATAAACTGC |
| 7621 | CCGTCGTTGG ATTGCGTGTT CAGGTTGGCG TACACGTGAC TGGGCACGGC TTCTTTTTTT |
| | GGCAGCAACC TAACGCACAA GTCCAACCGC ATGTGCACTG ACCCGTGCCG AAGAAAAAAA |
| 7681 | ACCACTATCG CATCTTCGTC GTACGCGGAT CTACAACCAA TCCCGTTGCC CACATAAGCG |
| | TGGTGATAGC GTAGAAGCAG CATGCGCCTA GATGTTGGTT AGGGCAACGG GTGTATTCGC |
| 7741 | TACGCGTTTA AAACGTGCGA TAGGTCTTTG GCCAATTCGC AATCAGCGTC CACTTTAACG |
| | ATGCGCAAAT TTTGCACGCT ATCCAGAAAC CGGTTAAGCG TTAGTCGCAG GTGAAATTGC |
| 7801 | TTGTTGCGTA ACTCGTTTAA AGCATTAATA ATGACGTCAT TTTCCGCATG ACAACTGGTT |
| | AACAACGCAT TGAGCAAATT TCGTAATTAT TACTGCAGTA AAAGGCGTAC TGTTGACCAA |
| 7861 | AGCTTGAAAA ACGGAACCGA GTAGTGGCAT GAATAAAATA AATCTTTGTT GTCTAATATT |
| | TCGAACTTTT TGCCTTGGCT CATCACCGTA CTTATTTTAT TTAGAAACAA CAGATTATAA |
| 7921 | GGGGGGGAGC TCTTGTGAGT CCTCGCGGGT AGGTACCACC ACCCTGCCTA TTTCTGCCGT |
| | CCCCCCCTCG AGAACACTCA GGAGCGCCCA TCCATGGTGG TGGGACGGAT AAAGACGGCA |
| 7981 | GAAGCAGTAA TGCGTTTCGG TTTGAAGAGT GGGGCGGCCG TGGTACTGAG ACCTTAGAAC |
| | CTTCGTCATT ACGCAAAGCC AAACTTCTCA CCCCGCCGGC ACCATGACTC TGGAATCTTG |
| 8041 | TCATATCTGA AGGTGGGTGG CACATTTACG TTGTAGATGT CTATGGGCTC CAGTAACCAC |
| | AGTATAGACT TCCACCCACC GTGTAAATGC AACATCTACA GATACCCGAG GTCATTGGTG |
| 8101 | TTAACATCAG GTGGGCTGTG AGCTCTTACA CCCATCTACG CAATAAAAAA TTAAAAATAA |
| | AATTGTAGTC CACCCGACAC TCGAGAATGT GGGTAGATGC GTTATTTTTT AATTTTTATT |
| 8161 | ATATGTTTGA AGTCCGTAAC ATAGATTCCG TATTTTTACA GTTGTTTTTC ACGTTTTTCA |
| | TATACAAACT TCAGGCATTG TATCTAAGGC ATAAAAATGT CAACAAAAAG TGCAAAAAGT |
| 8221 | TTTCTTCACC GACAATGGAA AATAATCACA CACAAATACA CTGTATAGTA ACAACGAGCA |
| | AAAGAAGTGG CTGTTACCTT TTATTAGTGT GTGTTTATGT GACATATCAT TGTTGCTCGT |
| 8281 | GAGCCGATTT TGGAGTTTCG ATAAAGCGAG GCTACCAAGA ATGCGGCAGA TAAGATTTAC |
| | CTCGGCTAAA ACCTCAAAGC TATTTCGCTC CGATGGTTCT TACGCCGTCT ATTCTAAATG |
| 8341 | GTACATTCAA GAGTCGCTGA TAACAACTTT TACCTCTCAA ATTGCCCACA GTGCGATCAC |
| | CATGTAAGTT CTCAGCGACT ATTGTTGAAA ATGGAGAGTT TAACGGGTGT CACGCTAGTG |
| 8401 | AAGAAACATA GACGAACGGA TCTGTGCGCA ACGAGCCGCT ACGATATCAT TATCATACAG |
| | TTCTTTGTAT CTGCTTGCCT AGACACGCGT TGCTCGGCGA TGCTATAGTA ATAGTATGTC |
| 8461 | ATTTTTATCT TTTCATCTAG CTTCAGTTAG TGATGCTTTC TGATCTCTTC ATAATTATAA |
| | TAAAAATAGA AAAGTAGATC GAAGTCAATC ACTACGAAAG ACTAGAGAAG TATTAATATT |
| 8521 | TTAAAAAGAA TAAATTATCT AGTAATATAG TTCTACTACG GTACACGAAT TTTGAGATTA |
| | AATTTTTCTT ATTTAATAGA TCATTATATC AAGATGATGC CATGTGCTTA AAACTCTAAT |
| 8581 | ATTAACCGGA TTTTCTGGGT TATGATTTAC ATCGGTACAG AATCTAGTGA AAGCACGTCG |
| | TAATTGGCCT AAAAGACCCA ATACTAAATG TAGCCATGTC TTAGATCACT TTCGTGCAGC |
| 8641 | AGTGAAATTC TATGAAACTT CGGCGGGAGT CGGGGAGAGG TTACAAGCGA CCGCGAGGTG |
| | TCACTTTAAG ATACTTTGAA GCCGCCCTCA GCCCCTCTCC AATGTTCGCT GGCGCTCCAC |
| 8701 | CCGCTAACTT AATCAGTTAT CAAGGCATCG CCTTATCAAA AGATGCGAGC TGATAGCGTG |
| | GGCGATTGAA TTAGTCAATA GTTCCGTAGC GGAATAGTTT TCTACGCTCG ACTATCGCAC |
| 8761 | CGCGTTACCA TATATGGTGA CAAAAACTGA GTCAGCCCGC GATTGGTGGA AAAACAAACT |
| | GCGCAATGGT ATATACCACT GTTTTTGACT CAGTCGGGCG CTAACCACCT TTTTGTTTGA |
| 8821 | GGAGCCGATA CTGTGTAAAT TGTGATAACG GCTCTTTTAT ATAGTTTATC CTCACGAGTC |
| | CCTCGGCTAT GACACATTTA ACACTATTGC CGAGAAAATA TATCAAATAG GAGTGCTCAG |
| 8881 | GGTTCTCATT TACTAAGGTG TGCTCGAACA GTGCGCATTC GCATCTACGT ACTTGTCACT |
| | CCAAGAGTAA ATGATTCCAC ACGAGCTTGT CACGCGTAAG CGTAGATGCA TGAACAGTGA |
| 8941 | TATTTAATAA TACTATGTAA GTTTTAATTT TAAAATTGCG AAAGAAAAAA AAACATATTT |
| | ATAAATTATT ATGATACATT CAAAATTAAA ATTTTAACGC TTTCTTTTTT TTTGTATAAA |
| 9001 | ATTTATTTGT AAAATTTGAA TTTCGAAGGT TCTCCGTCCC TTTACCTTTA AGTATTACAT |
| | TAAATAAACA TTTTAAACTT AAAGCTTCCA AGAGGCAGGG AAATGGAAAT TCATAATGTA |
| 9061 | ATGTTTGAGT GTTTTTTTTT TTTAATAATA CGCTAATGAT AACGTGTTAC GTTACATAAT |
| | TACAAACTCA CAAAAAAAAA AAATTATTAT GCGATTACTA TTGCACAATG CAATGTATTA |
| 9121 | TGTTGCATAA CTAGTGAAGT GAAATTTTTT ATAAAAAAAA ACATTTTTCG GAATTTAGTG |
| | ACAACGTATT GATCACTTCA CTTTAAAAAA TATTTTTTTT TGTAAAAAGC CTTAAATCAC |
| | PstI |
| | ˜˜˜˜˜˜˜ |
| 9181 | TACTGCAGAT GTTAATAAAC ACTACTAAAT AAGAAATAAG TTTATTGGAC GCACATTTCA |
| | ATGACGTCTA CAATTATTTG TGATGATTTA TTCTTTATTC AAATAACCTG CGTGTAAAGT |
| | ClaI |
| | ˜˜˜˜˜˜˜ |
| 9241 | AAGTGTCCAC TCGCATCGAT CAATTCGGAA ACAGAAATTG GGAACAGTGA ATTATGAATC |
| | TTCACAGGTG AGCGTAGCTA GTTAAGCCTT TGTCTTTAAC CCTTGTCACT TAATACTTAG |
| 9301 | TTATACAGTT TTCTTTAACG TCACTAAATA GATGGACGCA AATAAATTTG TCGTTTACTT |
| | AATATGTCAA AAGAAATTGC AGTGATTTAT CTACCTGCGT TTATTTAAAC AGCAAATGAA |
| 9361 | AGTATAATGT ATGGAATGAG AATGTAGTTT GAATTGTTTT TTTTCTTTTC TTGCAGACTA |
| | TCATATTACA TACCTTACTC TTACATCAAA CTTAACAAAA AAAAGAAAAG AACGTCTGAT |
| | HindIII |
| | ˜˜˜˜˜˜ |
| | ClaI |
| | ˜˜˜˜˜˜˜ |
| 9421 | ATTCAAGAGG TGCGACGAAG AAGTTGCCGC GTTGGTAGTA GACGGTATCG ATAAGCTTGA |
| | TAAGTTCTCC ACGCTGCTTC TTCAACGGCG CAACCATCAT CTGCCATAGC TATTCGAACT |
| | PstI |
| | ˜˜˜˜˜˜ |
| | EcoRI |
| | ˜˜˜˜˜˜˜ |
| 9481 | TATCGAATTC CTGCAGCCCT GTAATACGAC TCACTATAGG GCGAATTGGG TACCGGGCCC |
| | ATAGCTTAAG GACGTCGGGA CATTATGCTG AGTGATATCC CGCTTAACCC ATGGCCCGGG |
| | HindIII PstI BamHI |
| | ˜˜˜˜˜˜˜ ˜˜˜˜˜˜ ˜˜˜˜˜˜ |
| | SmaI |
| | ˜˜˜˜˜˜˜ |
| | XmaI |
| | ˜˜˜˜˜˜˜ |
| | AvaI ClaI EcoRI AvaI NcoI |
| | ˜˜˜˜˜˜ ˜˜˜˜˜˜˜ ˜˜˜˜˜˜˜ ˜˜˜˜˜˜˜ ˜˜ |
| 9541 | CCCCTCGAGG TCGACGGTAT CGATAAGCTT GATATCGAAT TCCTGCAGCC CGGGGGATCC |
| | GGGGAGCTCC AGCTGCCATA GCTATTCGAA CTATAGCTTA AGGACGTCGG GCCCCCTAGG |
| | NcoI PstI PstI |
| | ˜˜˜˜ ˜˜˜˜˜˜˜ ˜˜ |
| | M A R S L L L P L Q I L L L S L A L E T |
| 9601 | ATGGCAAGAT CCCTTCTCCT GCCCCTGCAG ATCCTACTGC TATCCTTAGC CTTGGAAACT |
| | TACCGTTCTA GGGAAGAGGA CGGGGACGTC TAGGATGACG ATAGGAATCG GAACCTTTGA |
| | PstI |
| | ˜˜˜˜ |
| | A G E E A Q G D K I I D G A P C A R G S |
| 9661 | GCAGGAGAAG AAGCCCAGGG TGACAAGATT ATTGATGGCG CCCCATGTGC AAGAGGCTCC |
| | CGTCCTCTTC TTCGGGTCCC ACTGTTCTAA TAACTACCGC GGGGTACACG TTCTCCGAGG |
| | NcoI |
| | ˜˜˜˜˜˜ |
| | H P W Q V A L L S G N Q L H C G G V L V |
| 9721 | CACCCATGGC AGGTGGCCCT GCTCAGTGGC AATCAGCTCC ACTGCGGAGG CGTCCTGGTC |
| | GTGGGTACCG TCCACCGGGA CGAGTCACCG TTAGTCGAGG TGACGCCTCC GCAGGACCAG |
| | ApaLI |
| | ˜˜˜˜˜˜ |
| | N E R W V L T A A H C K M N E Y T V H L |
| 9781 | AATGAGGGCT GGGTGCTCAC TGCCGCCCAC TGCAAGATGA ATGAGTACAC CGTGCACCTG |
| | TTACTCGCGA CCCACGAGTG ACGGCGGGTG ACGTTCTACT TACTCATGTG GCACGTGGAC |
| | G S D T L G D R R A Q R I K A S K S F R |
| 9841 | GGCAGTGATA CGCTGGGCGA CAGGAGAGCT CAGAGGATCA AGGCCTCGAA GTCATTCCGC |
| | CCGTCACTAT GCGACCCGCT GTCCTCTCGA GTCTCCTAGT TCCGGAGCTT CAGTAAGGCG |
| | H P G Y S T Q T H V N D L M L V K L N S |
| 9901 | CACCCCGGCT ACTCCACACA GACCCATGTT AATGACCTGA TGCTCGTGAA GCTCAATAGC |
| | GTGGGGCCGA TGAGGTGTGT CTGGGTACAA TTACTGGAGT ACGAGCACTT CGAGTTATCG |
| | NcoI |
| | ˜˜˜˜˜˜˜ |
| | Q A R L S S M V K K V R L P S R C E P P |
| 9961 | CAGGCCAGGC TGTCATCCAT GGTGAAGAAA GTCAGGCTGC CCTCCCGCTG CGAACCCCCT |
| | GTCCGGTCCG ACAGTAGGTA CCACTTCTTT CAGTCCGACG GGAGGGCGAC GCTTGGGGGA |
| | G T T C T V S G W G T T T S P D V T F P |
| 10021 | GGAACCACCT GTACTGTCTC CGGCTGGGGC ACTACCACGA GCCCAGATGT GACCTTTCCC |
| | CCTTGGTGGA CATGACAGAG GCCGACCCCG TGATGGTGCT CGGGTCTACA CTGGAAAGGG |
| | S D L M C V D V K L I S P Q D C T K V Y |
| 10081 | TCTGACCTCA TGTGCGTGGA TGTCAAGCTC ATCTCCCCCC AGGACTGCAC GAAGGTTTAC |
| | AGACTGGAGT ACACGCACCT ACAGTTCGAG TAGAGGGGGG TCCTGACGTG CTTCCAAATG |
| | K D L L E N S M L C A G I P D S K K N A |
| 10141 | AAGGACTTAC TGGAAAATTC CATGCTGTGC GCTGGCATCC CCGACTCCAA GAAAAACGCC |
| | TTCCTGAATG ACCTTTTAAG GTACGACACG CGACCGTAGG GGCTGAGGTT CTTTTTGCGG |
| | C N G D S G G P L V C R G T L Q G L V S |
| 10201 | TGCAATGGTG ACTCAGGGGG ACCGTTGGTG TGCAGAGGTA CCCTGCAAGG TCTGGTGTCC |
| | ACGTTACCAC TGAGTCCCCC TGGCAACCAC ACGTCTCCAT GGGACGTTCC AGACCACAGG |
| | W G T F P C G Q P N D P G V Y T Q V C K |
| 10261 | TGGGGAACTT TCCCTTGCGG CCAACCCAAT GACCCAGGAG TCTACACTCA AGTGTGCAAG |
| | ACCCCTTGAA AGGGAACGCC GGTTGGGTTA CTGGGTCCTC AGATGTGAGT TCACACGTTC |
| | NotI |
| | ˜˜˜˜˜˜˜˜ |
| | F T K W I N D T M K K H R G G R G G G G |
| 10321 | TTCACCAAGT GGATAAATGA CACCATGAAA AAGCATCGC |
| | AAGTGGTTCA CCTATTTACT GTGGTACTTT TTCGTAGCG |
| | G G G G G G T L F V A L Y D Y E A R T E |
| 10381 | AC ACTCTTTGTG GCCCTTTATG ACTATGAAGC ACGGACAGAA |
| | TG TGAGAAACAC CGGGAAATAC TGATACTTCG TGCCTGTCTT |
| | D D L S F H K G E K F Q I L N S S E G D |
| 10441 | GATGACCTGA GTTTTCACAA AGGAGAAAAA TTTCAAATAT TGAACAGCTC GGAAGGAGAT |
| | CTACTGGACT CAAAAGTGTT TCCTCTTTTT AAAGTTTATA ACTTGTCGAG CCTTCCTCTA |
| | W W E A R S L T T G E T G Y I P S N Y V |
| 10501 | TGGTGGGAAG CCCGCTCCTT GACAACTGGA GAGACAGGTT ACATTCCCAG CAATTATGTG |
| | ACCACCCTTC GGGCGAGGAA CTGTTGACCT CTCTGTCCAA TGTAAGGGTC GTTAATACAC |
| | NotI |
| | ˜˜˜˜˜˜˜˜ |
| | A P V D G G R G G G G G G H H H H H H * |
| 10561 | GCTCCAGTTG AC C ATCATCATCA TCATCATTAA |
| | CGAGGTCAAC TG G TAGTAGTAGT AGTAGTAATT |
| | BstXI |
| | ˜˜˜˜˜˜˜˜˜˜˜˜˜ |
| 10621 | CGCCACCGCG GTGGAGCTCC AGCTTTTGTT CCCTTTAGTG AGGGTTCGAG AAGTCTTACG |
| | GCGGTGGCGC CACCTCGAGG TCGAAAACAA GGGAAATCAC TCCCAAGCTC TTCAGAATGC |
| 10681 | AACTTCCCGA CGGTCAGGTC ATCACCATCG GAAACGAAAG ATTCCGTTGC CCAGAGGCCC |
| | TTGAAGGGCT GCCAGTCCAG TAGTGGTAGC CTTTGCTTTC TAAGGCAACG GGTCTCCGGG |
| 10741 | TCTTCCAACC CTCGTTCTTG GGTATGGAAG CCAACGGAAT CCACGAAACC ACATACAACT |
| | AGAAGGTTGG GAGCAAGAAC CCATACCTTC GGTTGCCTTA GGTGCTTTGG TGTATGTTGA |
| 10801 | CCATCATGAA GTGCGACGTG GACATCCGTA AGGACTTGTA CGCCAACACC GTATTGTCCG |
| | GGTAGTACTT CACGCTGCAC CTGTAGGCAT TCCTGAACAT GCGGTTGTGG CATAACAGGC |
| 10861 | GTGGTACCAC CATGTACCCT GGAATCGCCG ACCGTATGCA AAAGGAAATC ACACGTCTCG |
| | CACCATGGTG GTACATGGGA CCTTAGCGGC TGGCATACGT TTTCCTTTAG TGTGCAGAGC |
| 10921 | CCCCATCGAC AATGAAGATT AAGATCATCG CTCCCCCAGA GAGGAAGTAC TCCGTATGGA |
| | GGGGTAGCTG TTACTTCTAA TTCTAGTAGC GAGGGGGTCT CTCCTTCATG AGGCATACCT |
| | ClaI |
| | ˜˜˜˜˜˜˜ |
| 10981 | TCGGTGGATC GATCCTCGCC TCCCTCTCTA CCTTCCAACA GATGTGGATC TCGAAACAGG |
| | AGCCACCTAG CTAGGAGCGG AGGGAGAGAT GGAAGGTTGT CTACACCTAG AGCTTTGTCC |
| 11041 | AGTACGACGA GTCTGGTCCC TCCATTGTAC ACAGGAAGTG CTTCTAAGCG TTGAGACTTT |
| | TCATGCTGCT CAGACCAGGG AGGTAACATG TGTCCTTCAC GAAGATTCGC AACTCTGAAA |
| 11101 | AAGTTATGAT GCCCTACAGC AGAACCTCAA GAGGGTGGCT CAAATTACGC TTGTGATCTT |
| | TTCAATACTA CGGGATGTCG TCTTGGAGTT CTCCCACCGA GTTTAATGCG AACACTAGAA |
| 11161 | GTAAATAAAT TCAGTATTTA ATGTAGGTTG TAAGGTATTG TAATATGCAT ATTACGTAAA |
| | CATTTATTTA AGTCATAAAT TACATCCAAC ATTCCATAAC ATTATACGTA TAATGCATTT |
| 11221 | ACGAACGGAA TGTTGTTGTT GCCGTTTTTT TTTTGACAAA GATTTTTATT TATTAAAGTT |
| | TGCTTGCCTT ACAACAACAA CGGCAAAAAA AAAACTGTTT CTAAAAATAA ATAATTTCAA |
| 11281 | ACTAACCCCA AAACTTTTTA ATAAAATAAA TTTATATACC GGTATAATAA CTGACGTTTT |
| | TGATTGGGGT TTTGAAAAAT TATTTTATTT AAATATATGG CCATATTATT GACTGCAAAA |
| | ApaLI |
| | ˜˜˜˜˜˜˜ |
| 11341 | TCACTTGCTG TCCCCGCTCC CGACTAACAG TACGTCGTGT GCACCGAAAT TACCGATTTC |
| | AGTGAACGAC AGGGGCGAGG GCTGATTGTC ATGCAGCACA CGTGGCTTTA ATGGCTAAAG |
| 11401 | GTACACCGTT TGAGACAGTT ACGCTAGGAG CACAAATCTC CCAGCTGCAT ACCGTTGTTT |
| | CATGTGGCAA ACTCTGTCAA TGCGATCCTC GTGTTTAGAG GGTCGACGTA TGGCAACAAA |
| | PstI PstI |
| | ˜˜˜˜˜˜ ˜˜˜˜˜˜˜ |
| 11461 | ACTGCAGCTC TGCAGTCTTT AATTGGAATG CGAGTCGTTG ACCGCTTAAT ACGAAACATT |
| | TGACGTCGAG ACGTCAGAAA TTAACCTTAC GCTCAGCAAC TGGCGAATTA TGCTTTGTAA |
| 11521 | CTAAAATTCG CAAAATGCAA AGGAAACTGG TTCTGTACTT TCTACCTTTC AAAAGATTCA |
| | GATTTTAAGC GTTTTACGTT TCCTTTGACC AAGACATGAA AGATGGAAAG TTTTCTAAGT |
| 11581 | CCAAATTAAT TTTATGCGGA CTCACTAATT CCGTAGAAAT CTGTGTAGAG GTACCCAGGT |
| | GGTTTAATTA AAATACGCCT GAGTGATTAA GGCATCTTTA GACACATCTC CATGGGTCCA |
| 11641 | TACGCTTAGG CATAAGATGA CTGTTCGCGT TTTTACAATA CATACGAGCA GGTTACACAC |
| | ATGCGAATCC GTATTCTACT GACAAGCGCA AAAATGTTAT GTATGCTCGT CCAATGTGTG |
| 11701 | AAGATGAACA TCCTTTGATG CGTCTGTGTC TTGACCCGTC TGAGATTTGA GTGACTTGTC |
| | TTCTACTTGT AGGAAACTAC GCAGACACAG AACTGGGCAG ACTCTAAACT CACTGAACAG |
| 11761 | AACGTCATTG CGTAGTGTCA CCGGTCGTCG AGATCCCCGC CGCGGTGGAG CTACGAGCTC |
| | TTGCAGTAAC GCATCACAGT GGCCAGCAGC TCTAGGGGCG GCGCCACCTC GATGCTCGAG |

SCCE Production
H5 Transfection Protocol and Purification
   Transfection
1. High 5 insect cells (Invitrogen) are grown in monolayer in T-75 flasks in Express 5 media (Invitrogen) and adapted to ESF-921 media (Expression Systems) at 27° C. in a non-humidified, non-CO2 environment. Gentamycin is added at 10 ug/mL. Passage is by sloughing or squirting media over cells to loosen them at 1:3-1:5, when confluent.
2. Cells are then adapted to suspension and are grown in baffle shake flasks at 27° C. and 125 rpm. Cell density is maintained between 5×10⁵ and 3×10⁶.
3. Cells are transfected with SCCE sequence (or other suitable protein) containing the secretion signal peptide, and a 6-Histidine tag, previously cloned into in the pIE1-153A V4+plasmid vector (Cytostore). Plasmid DNA is purified using Qiagen Endo-Free purification Kits. The DNA is heat inactivated at 65° C. for 15 min prior to use.
4. On the day of transfection, cells are counted and checked for viability by trypan blue exclusion. Viability should exceed 95%.
5. For each 50 mL final transfection volume, 1.5×10⁷ cells are needed. The appropriate volume of culture based on the cell count is centrifuged at 800×g for 5 minutes, the supernatant aspirated and the pelleted cells immediately resuspended in 10 mL (1:5 volume) antibiotic free ESF-921 media.
6. For each 50 mL final transfection volume, 25 ug of vector DNA is diluted in 0.5 mL final volume of 0.15 M NaCl. In a separate tube, 50 uL of linear polyethylene imine (PEI MW=25,000, Polysciences) 1 mg/mL (sterile filtered and pH adjusted to 7.0), is also diluted into 0.5 mL of 0.15 M NaCl. Steps 4, 5 & 6 can be scaled up accordingly.
7. The two tubes containing the DNA and the PEI are then mixed, briefly vortexed, and allowed to incubate at room temp. for 5-10 minutes to form complexes.
8. The complexes are then added to the resuspended High 5 cells and the transfection mixture is placed on a gentle rocking platform (2-4 agitations per minute) for 5 hours at room temperature.
9. The transfection mixture is incubated at room temp with gentle agitation for five hours and then diluted 1:5 into baffled shake flasks containing ESF921. Supplement the ESF-921 with additional L-Glutamine at 2 mM final. Also add Penicillin-Streptomycin at 100-200 U/mL, 100 ug/mL and Amphotericin B (Fungizone, Invitrogen) at 0.25 ug/mL.
10. The cultures are shaken at 27° C. 125 rpm, for 6 days.
   Purification
1. The His-tagged proteins were purified by capture and elution from Ni-NTA agarose (Qiagen) according to the manufacturer's protocol.
2. Cultures are harvested, by centrifugation at 2000×g for 15 minutes and the media containing the secreted protein collected.
3. Centricon −70 (Millipore) 10 Kd MWCO filters are used to concentrate the protein. The cell supernatant is spun at 3500×g until the volumes are reduced by 20-50 fold.
4. The concentrated protein is diluted 10-20× with Ni-NTA lysis buffer (50 mM NaH₂PO₄, 300 mM NaCl, 10 mM Imidazole, pH8.0).
5. For each 50 mL of diluted protein solution 1 mL of Ni-NTA agarose (50% slurry) is used. Prior to use, the Ni-NTA agarose is pre-washed with 10 volumes lysis buffer, spun at 1000×g for 5 min. and resuspended in its original volume.
6. The Ni-NTA agarose is added to the protein solution and allowed to incubate in batch on a rotator for 2-18 hours at 4° C.
7. After incubation, mixture is centrifuged at 1000×g for 5 min. and most of the supernatant except 5 mL is removed.
8. The remaining supernatant and agarose slurry is transferred to a 10-20 mL chromatography Readi-column (Biorad) and the matrix allowed to settle at 4° C. Any pipets used and tubes are rinsed to collect additional beads and this is also transferred to the column.
9. The remaining supernatant is allowed to drain, and the column is washed with 10-12 volumes of Ni-NTA lysis buffer (50 mM NaH₂PO₄, 300 mM NaCl, 10 mM Imidazole, pH8.0)
10. The protein is eluted by addition of Ni-NTA elution buffer (50 mM NaH₂PO₄, 300 mM NaCl, 250 mM Imidazole, pH8.0). An initial fraction of 1.5 mL is collected, and 2-3 additional 1 mL fractions are also collected. Generally, the majority of the protein is present in fraction 1.

11. The fractions along with the supernatant and wash can be analyzed by SDS—PAGE and western blotting using the Penta-His antibody (Qiagen) or a protein specific antibody.

6 mer R4 SCCE WT sequences

| MP | 6 mer Lib Panning Round 4 SCCE WT |
|---|---|
| # | Hypervarible Domain |
| 040207_1 | TGC CCT GTG GCG GAG ACG CCT TGC |
| | Pro val ala glu thr pro |
| 040207_3 | TGC ACT GCT CAG CGG GTG GAT TGC |
| | Thr ala gln arg val asp |
| 040207_4 | TGC ACT GCT CAG CGG GTG GAT TGC |
| | Thr ala gln arg val asp |
| 040207_5 | TGC AGT CAT GTT AGG CGT AAT TGC |
| | Ser his val arg arg asn |
| 040907_1 | TGC AAG AGG AAT AAT AAG ATG TGC |
| | Lys arg asn asn lys met |
| 040907_3 | TGC ACT AAG CGT ACG ACT ATT TGC |
| | Thr lys arg thr thr ile |
| 040907_5 | TGC CCT TGG CAG CCT TGT CCT TGC |
| | Pro trp gln pro cys pro |
| 040907_7 | TGC GAG CAT ATG AAT AAG AGT TGC |
| | Asp his met asn lys ser |
| 040907_8 | TGC CCG AGG CAG AAT AAG TGT TGC |
| | Pro arg gln asn lys cys |
| 041307_2 | TGC AAG CGG TTG ATG TCG AAG TGC |
| | Lys Arg Leu Met Ser lys |
| 041307_3 | TGC CAG CCG CAT ACG TGG AAG TGC |
| | Gln Pro His Thr Trp Lys |
| | (Also in SCCE FYN) |
| 041307_4 | TGC ACG GCT GCG GTG GAT CAG TGC |
| | Thr Ala Ala Val Asp Gln |
| 041307_5 | TGC AAG CAG AAT AGT GAG GCG TGC |
| | Lys Gln Asn Ser Glu Ala |
| 041307_7 | TGC CCT GTG GCG GAG ACG CCT TGC |
| | Pro Val Ala Glu Thr Pro |
| 041307_8 | TGC ACG CCT AAT TCT GCG ATT TGC |
| | Thr Pro Asn Ser Ala Ile |
| 041307_10 | TGC AGT CAT GTT AGG CGT AAT TGC |
| | Ser His Val Arg Arg Asn |
| 041307_11 | TGC CAT CAT GGG CTT ATT GTG TGC |
| | His His Gly Leu Ile Val |
| 041307_12 | TGC TAT GCG AAG ACG ATG CGG TGC |
| | Tyr Ala Lys Thr Met Arg |
| 041307_17 | TGC CAT CAT GGG CTT ATT GTG TGC |
| | His His Gly Leu Ile Val |
| 041307_18 | TGC CAT CAT GGG CTT ATT GTG TGC |
| | His His Gly Leu Ile Val |
| 041307_19 | TGC CAT CAT GGG CTT ATT GTG TGC |
| | His His Gly Leu Ile Val |
| 041307_22 | TGC CAT CAT GGG CTT ATT GTG TGC |
| | His His Gly Leu Ile Val |
| 041307_25 | TGC ACT CCT CTG GCG CTT CCT TGC |
| | Thr Pro Lys Ala Lys Pro |
| 041307_27 | TGC AAG AAG AAG AAG ACG AAG TGC |
| | Lys Lys Lys Lys Thr Lys |
| 041307_29 | TGC CAT CAT GGG CTT ATT GTG TGC |
| | His His Gly Leu Ile Val |
| 041307_30 | TGC CCG AAT AAT AAG ATT AGG TGC |
| | Pro Asn Asn Lys Ile Arg |
| 041307_31 | TGC ACT TCT ACT AGG CCT CCT TGC |
| | Thr Ser Thr Arg Pro Pro |
| 041307_32 | TGC CAT ATG AAT ATG TAT ATT TGC |
| | His Met Asn Met Tyr Ile |
| 041307_35 | TGC ACG GGG GCG GGG CGG TCG TGC |
| | Thr Gly Ala Gly Arg Ser |

6 mer R4 SCCE Fyn sequences

| MP | 6 mer Lib Panning Round 4 SCCE FYN |
|---|---|
| # | Hypervarible Domain |
| 040207_10 | TGC ATG CCG CAT AAG AAG GAT TGC |
| | Met pro his lys lys asp |
| 040607_1 | TGC CCT TCT GTG TAT AAG CAG TGC |
| | Pro ser val tyr lys gln |
| 040607_2 | TGC CCT TCT GTG TAT AAG CAG TGC |
| | Pro ser val tyr lys gln |
| 040607_3 | TGC CAG CCC CAT ACG TGG AAG TGC |
| | Gln pro his thr trp lys |
| | (!!Also in SCCE WT!!) |
| 040607_5 | TGC ACG ACT ACG ATG TCT GCT TGC |
| | Thr thr thr met ser ala |
| 040607_6 | TGC AGG CAT AAG AGT AAG AAT TGC |
| | Arg his lys ser lys asn |

Attention is drawn to the following references:
http://www.biosci.missouri.edu/smithgp/PhageDisplayWebsite/PhageDispltyWebsiteIndex.html; Phage Display A Laboratory Manual, Carlos Barbas III [et al], 2001 Cold Spring Harbor Laboratory Press, Cold Spring Harbor, N.Y.; Phage Display-A Practical Approach, eds. T. Clackson and H. B. Lowman, 2004 Oxford University Press, Oxford, UK; The Manual for the pSKAN system MoBiTech; The teachings all references cited are incorporated herein by reference.

What is claimed is:

1. A method of obtaining a primary-result peptide having at least one binding domain that
binds a predetermined dynamic target material at a non-active site
wherein said dynamic target material has at least two conformational energy-minima states comprising:
(a) accessibly-conformationally restraining said dynamic target material in substantially a single conformational energy-minima state
(b) affinity-exposing said accessibly-conformationally restrained single conformational energy-minima dynamic target material to a peptide library comprising inquiry-peptides and identifying peptide which associate with the target with sufficient affinity to withstand washing at least about 4 times in rapid succession with a standard buffer containing physiologically balanced salt solution and a non-ionic detergent (<0.1% v/v) (“peptide hits”).
(c) affinity-exposing said accessible conformationally-restrained single conformational energy-minima state dynamic target material to said peptide library wherein said single conformational energy-minima state is substantially a single energy-minima state other than the state of step (a) and identifying peptide-hits; and
(d) selecting at least one peptide-hit that inhibits target function by other-than-competitive inhibition the target material, which peptide-hit being a primary-result peptide.

2. A method of obtaining a primary-result peptide having at least one binding domain wherein said binding domain is a low affinity binding domain comprising:
(a) preparing a target polypeptide, as a fusion protein having a known target region and an inquiry target region wherein the known target region is linked to the inquiry target region by a flexible linker;
(b) preparing a tandem peptide display library where said tandem peptides comprise
(i) a known peptide element having a binding domain of low affinity as to said known target region said element connected to
(ii) a flexible linker said flexible linker connected to
(iii) an inquiry peptide sequence
(c) affinity exposing said target protein to said peptide library;
(d) identifying tandem peptide-hits;
(e) identifying said inquiry peptide sequence of said tandem peptide hit as a primary result peptide.

3. The method of claim 2 wherein the known target region of (a) comprises an SH3 domain and the known peptide of step (b)(i) comprises a prolein-rich SH3 binding domain having an affinity for the known target region with an affinity in the range of 100 micromolar, so as to be of sufficiently low affinity to substantially dissociate from the known target region after washing at most about 4 times in rapid succession with a standard buffer containing physiologically balanced salt solution and a non-ionic detergent (<0.1% v/v).

4. The method of claim 2 wherein the flexible linker of step (b)(ii) is a short peptide.

5. A method of obtaining a primary-result peptide useful in inducing formation of activated-like multiprotein complexes bridging two partner polypeptides comprising:
(a) anchoring to a substratum a target polypeptide having a known dimerizable target region, said anchoring being at a location other than said target region and assembling the multiprotein complex, as a ternary complex, by adding a partner target polypeptide and cognate-like accessory polypeptide which bridges the two partner polypeptide targets;
(b) exposing said substratum anchored activated-like multiprotein complex to a phage peptide display library and
(c) selecting phage that bind the assembled protein-protein complex with sufficient affinity to withstand washing four times in rapid succession with a standard buffer containing physiologically balanced salt solution and a non-ionic detergent (<0.1% v/v)
(d) selecting from among said complex binding phage a phage that when added to a system containing a substratum anchored target polypeptide and a partner target polypeptide, is capable of inducing the formation of the multiprotein complex such that the two target polypeptide partners become associated in the absence of the accessory polypeptide, said phage bearing a primary result peptide.

6. A method of preparing an enhanced peptide display library comprising
preparing a tandem peptide display library having a known target region and an inquiry target region where said tandem peptides comprise
(i) a known peptide element having a binding domain of low affinity as to said known target region said element connected to
(ii) a flexible linker said flexible linker connected to
(iii) an inquiry peptide sequence
(iii) wherein said inquiry peptide sequence is further connected to a bacteriophage structural protein.

7. A library of the method of claim 6.

8. An enhanced peptide display library comprising a tandem peptide display library having a known target region and an inquiry target region where said tandem peptides comprise
(i) a known peptide element having a binding domain of low affinity as to said known target region said element connected to
(ii) a flexible linker said flexible linker connected to
(iii) an inquiry peptide sequence
(iii) wherein said inquiry peptide sequence is further connected to a bacteriophage structural protein.
